SEQUENCE LISTING (127 sequences)

SEQ ID NO: 1 - DNA, 749 bp, Homo sapiens
  misc_feature: sequence of STAR1
atgcggtgggggcgcgccagagactcgtgggatccttggcttggatgtttggatctttctgagttgcctgtgccgcgaaagacaggtacatttctgattaggcctgtgaagcctcctggaggaccatctcattaagacgatggtattggagggagagtcacagaaagaactgtggcccctccctcactgcaaaacggaagtgattttattttaatgggagttggaatatgtgagggctgcaggaaccagtctccctccttcttggttggaaaagctggggctggcctcagagacaggttttttggccccgctgggctgggcagtctagtcgaccctttgtagactgtgcacacccctagaagagcaactacccctatacaccaggctggctcaagtgaaaggggctctgggctccagtctggaaaatctggtgtcctggggacctctggtcttgcttctctcctcccctgcactggctctgggtgcttatctctgcagaagcttctcgctagcaaacccacattcagcgccctgtagctgaacacagcacaaaaagccctagagatcaaaagcattagtatgggcagttgagcgggaggtgaatatttaacgcttttgttcatcaataactcgttggctttgacctgtctgaacaagtcgagcaataaggtgaaatgcaggtcacagcgtctaacaaatatgaaaatgtgtatattcaccccggtctccagccggcgcgccaggctccc

SEQ ID NO: 2 - DNA, 883 bp, Homo sapiens
  misc_feature: sequence of STAR2
gggtgcttcctgaattcttccctgagaaggatggtggccggtaaggtccgtgtaggtggggtgcggctccccaggccccggcccgtggtggtggccgctgcccagcggcccggcacccccatagtccatggcgcccgaggcagcgtgggggaggtgagttagaccaaagagggctggcccggagttgctcatgggctccacatagctgccccccacgaagacggggcttccctgtatgtgtggggtcccatagctgccgttgccctgcaggccatgagcgtgcgggtcatagtcgggggtgccccctgcgcccgcccctgccgccgtgtagcgcttctgtgggggtggcgggggtgcgcagctgggcagggacgcagggtaggaggcggggggcagcccgtaggtaccctgggggggcttggagaagggcgggggcgactggggctcatacgggacgctgttgaccagcgaatgcatagagttcagatagccaccggctccggggggcacggggctgcgacttggagactggccccccgatgacgttagcatgcccttgcccttctgatcctttttgtacttcatgcggcgattctggaaccagatcttgatctggcgctcagtgaggttcagcagattggccatctccacccggcgcggccggcacaggtagcggttgaagtggaactctttctccagctccaccagctgcgcgctcgtgtaggccgtgcgcgcgcgcttggacgaagcctgccccggcgggctcttgtcgccagcgcagctttcgcctgcgaggacagagagaggaagagcggcgtcaggggctgccgcggccccgcccagcccctgacccagcccggcccctccttccaccaggccccaa

SEQ ID NO: 3 - DNA, 2126 bp, Homo sapiens
  misc_feature: sequence of STAR3
atctcgagtactgaaataggagtaaatctgaagagcaaataagatgagccagaaaaccatgaaaagaacagggactaccagttgattccacaaggacattcccaaggtgagaaggccatatacctccactacctgaaccaattctctgtatgcagatttagcaaggttataaggtagcaaaagattagacccaagaaaatagagaacttccaatccagtaaaaatcatagcaaatttattgatgataacaattgtctccaaaggaacaaggcagagtcgtgctagcagaggaagcacgtgagctgaaaacagccaaatctgctttgttttcatgacacaggagcataaagtacacaccaccaactgacctattaaggctgtggtaaaccgattcatagagagaggttctaaatacattggtccctcacaggcaaactgcagttcgctccgaacgtagtccctggaaatttgatgtccagtatagaaaagcagagcagtcaaaaaatatagataaagctgaaccagatgttgcctgggcaatgttagcagcaccacacttaagatataacctcaggctgtggactccctccctggggagcggtgctgccggcggcgggcgggctccgcaactccccggctctctcgcccgccctcccgttctcctcgggcggcggcgggggccgggactgcgccgctcacagcggcggctcttctgcgcccggcctcggaggcagtggcggtggcggccatggcctcctgcgttcgccgatgtcagcatttcgaactgagggtcatctccttgggactggttagacagtgggtgcagcccacggagggcgagttgaagcagggtggggtgtcacctcccccaggaagtccagtgggtcagggaactccctcccctagccaagggaggccgtgagggactgtgcccggtgagagactgtgccctgaggaaaggtgcactctggcccagatactacacttttcccacggtcttcaaaacccgcagaccaggagattccctcgggttcctacaccaccaggaccctgggtttcaaccacaaaaccgggccatttgggcagacacccagctagctgcaagagttgtttttttttttatactcctgtggcacctggaacgccagcgagagagcacctttcactcccctggaaagggggctgaaggcagggacctttagctgcgggctagggggtttggggttgagtgggggaggggagagggaaaaggcctcgtcattggcgtcgtctgcagccaataaggctacgctcctctgctgcgagtagacccaatcctttcctagaggtggagggggcgggtaggtggaagtagaggtggcgcggtatctaggagagagaaaaagggctggaccaataggtgcccggaagaggcggacccagcggtctgttgattggtattggcagtggaccctcccccggggtggtgccggagggggggatgatgggtcgaggggtgtgtttatgtggaagcgagatgaccggcaggaacctgccccaatgggctgcagagtggttagtgagtgggtgacagacagacccgtaggccaacgggtggccttaagtgtctttggtctcctccaatggagcagcggcggggcgggaccgcgactcgggtttaatgagactccattgggctgtaatcagtgtcatgtcggattcatgtcaacgacaacaacagggggacacaaaatggcggcggcttagtcctacccctggcggcggcggcagcggtggcggaggcgacggcactcctccaggcggcagccgcagtttctcaggcagcggcagcgcccccggcaggcgcggtggcggtggcgcgcagccaggtctgtcacccaccccgcgcgttcccagggggaggagactgggcgggaggggggaacagacggggggggattcaggggcttgcgacgcccctcccacaggcctctgcgcgagggtcaccgcggggccgctcggggtcaggctgcccctgagcgtgacggtagggggcgggggaaaggggaggagggacaggccccgcccctcggcagggcctctagggcaagggggcggggctcgaggagcggaggggggcggggcgg

SEQ ID NO: 4 - DNA, 1625 bp, Homo sapiens
  misc_feature: sequence of STAR4
gatctgagtcatgttttaaggggaggattcttttggctgctgagttgagattaggttgagggtagtgaaggtaaaggcagtgagaccacgtaggggtcattgcagtaatccaggctggagatgatggtggttcagttggaatagcagtgcatgtgctgtaacaacctcagctgggaagcagtatatgtggcgttatgacctcagctggaacagcaatgcatgtggtggtgtaatgaccccagctgggtagggtgcatgtggtgtaacgacctcagctgggtagcagtgtgtgtgatgtaacaacctcagctgggtagcagtgtacttgataaaatgttggcatactctagatttgttatgagggtagtgccattaaatttctccacaaattggttgtcacgtatgagtgaaaagaggaagtgatggaagacttcagtgcttttggcctgaataaatagaagacgtcatttccagttaatggagacagggaagactaaaggtagggtgggattcagtagagcaggtgttcagttttgaatatgatgaactctgagagaggaaaaactttttctacctcttagtttttgtgactggacttaagaattaaagtgacataagacagagtaacaagacaaaaatatgcgaggttatttaatatttttacttgcagaggggaatcttcaaaagaaaaatgaagacccaaagaagccattagggtcaaaagctcatatgcctttttaagtagaaaatgataaattttaacaatgtgagaagacaaaggtgtttgagctgagggcaataaattgtgggacagtgattaagaaatatatgggggaaatgaaatgataagttattttagtagatttattcttcatatctattttggcttcaacttccagtctctagtgataagaatgttcttctcttcctggtacagagagagcacctttctcatgggaaattttatgaccttgctgtaagtagaaaggggaagatcgatctcctgtttcccagcatcaggatgcaaacatttccctccattccagttctcaaccccatggctgggcctcatggcattccagcatcgctatgagtgcacctttcctgcaggctgcctcgggtagctggtgcactgctaggtcagtctatgtgaccaggagctgggcctctgggcaatgccagttggcagcccccatccctccactgctgggggcctcctatccagaagggcttggtgtgcagaacgatggtgcaccatcatcattccccacttgccatctttcaggggacagccagctgctttgggcgcggcaaaaaacacccaactcactcctcttcaggggcctctggtctgatgccaccacaggacatccttgagtgctgggcagtctgaggacagggaaggagtgatgaccacaaaacaggaatggcagcagcagtgacaggaggaagtcaaaggcttgtgtgtcctggccctgctgagggctggcgagggccctgggatggcgctcagtgcctggtcggctgcaagaggccagccctctgcccatgaggggagctggcagtgaccaagctgcactgccctggtggtgcatttcctgccccactctttccttctaagatcc

SEQ ID NO: 5 - DNA, 1571 bp, Homo sapiens
  misc_feature: sequence of STAR5
cacctgatttaaatgatctgtctggtgagctcactgggtctttactcgcatgctgggtccacagctccactgtcctgcagggtccgtgagtgtgggccccttatctatttcatcatcataaccctgcgtgtcctcaactcctggcacatattgggtggccccatccacacacggttgttgagtgaatccatgagatgacaaaggctatgatgtagactatatcatgagccagaaccaggctttcctacctccagacaatcaagggccttgatttgggattgagggagaaaggagtagaagccaggaaggagaagagattgaggtttaccaagggtgcaaagtcctggcccctgactgtaggctgaaaactatagaaatgatagaacaattttgcaatgaaatgcagaagaccctgcatcaactttaggtgggacttcgggtatttttatggccacagaacatcctcccatttacctgcatggcccagacacagacttcaaaacagttgaggccagcaggctccaggtaagtggtaggattccagaatgccctcagagtgttgtgggaggcagcaggcgattttcctggacttctgagtttatgagaaccccaaaccccaattggcattaacattgaggtctcaatgtatcatggcaggaagcttccgagtggtgaaaaggaaagtgaacatcaaagctcggaagacaagagggtggagtgatggcaaccaagagcaagacccttccctctcctgtgatggggtggctctatgtgaagcccccaaactggacacaggtctggcagaatgaggaacccactgagatttagcgccaacatccagcataaaagggagactgacatagaatttgagttagttaaaaataaggcacaatgcttttcatgtattcctgagttttgtggactggtgttcaatttgcagcattcttagttgattaaatctgagatgaagaaagagtgtccaacactttcaccttggaaagctctggaaaagcaaaagggagagacaattagcttcatccattaactcacttagtcattatgcattcattcatgtaactaccaaacacgtactgagtgcctaacactcctgagacactgagaagtttcttgggaatacaaagatgaataaaaaccacgccaggcaggagttggaggaaggttctggatgccaccacgctctacctcctggctggacaccaggcaatgttggtaaccttctgcctccaatttctgcaaatacataattaataaacacaaggttatcttctaaacagttcttaaaatgagtcaactttgtttaaacttgttctttttagagaaaaatgtatttttgaaagagttggttagtgctaggggaaatgtctgggcacagctcagtctggtgtgagagcaggaagcagctctgtgtgtctggggtgggtacgtatgtaggacctgtgggagaccaggttgggggaaggcccctcctcatcaagggctcctttgctttggtttgctttggcgtgggaggtgctgtgccacaagggaatacgggaaataagatctctgct

SEQ ID NO: 6 - DNA, 1173 bp, Homo sapiens
  misc_feature: sequence of STAR6
tgacccaccacagacatcccctctggcctcctgagtggtttcttcagcacagcttccagagccaaattaaacgttcactctatgtctatagacaaaaagggttttgactaaactctgtgttttagagagggagttaaatgctgttaactttttaggggtgggcgagagggatgacaaataacaacttgtctgaatgttttacatttctccccactgcctcaagaaggttcacaacgaggtcatccatgataaggagtaagacctcccagccggactgtccctcggcccccagaggacactccacagagatatgctaactggacttggagactggctcacactccagagaaaagcatggagcacgagcgcacagagcagggccaaggtcccagggacagaatgtctaggagggagattggggtgagggtaatctgatgcaattactgtggcagctcaacattcaagggagggggaagaaagaaacagtccctgtcaagtaagttgtgcagcagagatggtaagctccaaaatttgaaactttggctgctggaaagttttagggggcagagataagaagacataagagactttgagggtttactacacactagacgctctatgcatttatttatttattatctcttatttattactttgtataactcttataataatcttatgaaaacggaaaccctcatatacccattttacagatgagaaaagtgacaattttgagagcatagctaagaatagctagtaagtaaaggagctgggacctaaaccaaaccctatctcaccagagtacacactctttttttttttccagtgtaattttttttaatttttattttactttaagttctgggatacatgtgcagaaggtatggtttgttacataggtatatgtgtgccatagtggattgctgcacctatcaacccgtcatctaggtttaagccccacatgcattagctatttgtcctgatgctctccctcccctccccacaccagacaggccttggtgtgtgatgttcccctccctgtgtccatgtgttctcactgttcagctcccacttatgagtgagaacgtgtggtatttggttttctgttcctgtgttagtttgctgaggatgatggcttccagcttcatccatgtccctgcaaaggacacgatc

SEQ ID NO: 7 - DNA, 2101 bp, Homo sapiens
  misc_feature: sequence of STAR7
aggtgggtggatcacccgaggtcaggagttcaagaccagcctggccaacatggtaaaacctcgtctctactaaaaaatacgaaaaattagctggttgtggtggtgcgtgcttgtaatcccagctactcgggaggctgaggcaggagaatcacttgaatctgggaggcagaggttgcagtgagctgagatagtgccattgcactccagcctgggcaacagacggagactctgtctccaaaaaaaaaaaaaaaaatcttagaggacaagaatggctctctcaaacttttgaagaaagaataaataaattatgcagttctagaagaagtaatggggatataggtgcagctcatgatgaggaagacttagcttaactttcataatgcatctgtctggcctaagacgtggtgagctttttatgtctgaaaacattccaatatagaatgataataataatcacttctgaccccccttttttttcctctccctagactgtgaagcagaaaccccatatttttcttagggaagtggctacgcactttgtatttatattaacaactaccttatcaggaaattcatattgttgcccttttatggatggggaaactggacaagtgacagagcaaaatccaaacacagctggggatttccctcttttagatgatgattttaaaagaatgctgccagagagattcttgcagtgttggaggacatatatgacctttaagatattttccagctcagagatgctatgaatgtatcctgagtgcatggatggacctcagttttgcagattctgtagcttatacaatttggtggttttctttagaagaaaataacacatttataaatattaaaataggcccaagaccttacaagggcattcatacaaatgagaggctctgaagtttgagtttgttcactttctagttaattatctcctgcctgtttgtcataaatgcgtttagtagggagctgctaatgacaggttcctccaacagagtgtggaagaaggagatgacagctggcttcccctctgggacagcctcagagctagtggggaaactatgttagcagagtgatgcagtgaccaagaaaatagcactaggagaaagctggtccatgagcagctggtgagaaaaggggtggtaatcatgtatgccctttcctgttttattttttattgggtttccttttgcctctcaattccttctgacaatacaaaatgttggttggaacatggagcacctggaagtctggttcattttctctcagtctcttgatgttctctcgggttcactgcctattgttctcagttctacacttgagcaatctcctcaatagctaaagcttccacaatgcagattttgtgatgacaaattcagcatcacccagcagaacttaggtttttttctgtcctccgtttcctgacctttttcttctgagtgctttatgtcacctcgtgaaccatcctttccttagtcatctacctagcagtcctgattcttttgacttgtctccctacaccacaataaatcactaattactatggattcaatccctaaaatttgcacaaacttgcaaatagattacgggttgaaacttagagatttcaaacttgagaaaaaagtttaaatcaagaaaaatgacctttaccttgagagtagaggcaatgtcatttccaggaataattataataatattgtgtttaatatttgtatgtaacatttgaataccttcaatgttcttatttgtgttattttaatctcttgatgttactaactcatttggtagggaagaaaacatgctaaaataggcatgagtgtcttattaaatgtgacaagtgaatagatggcagaaggtggattcatattcagttttccatcaccctggaaatcatgcggagatgatttctgcttgcaaataaaactaacccaatgaggggaacagctgttcttaggtgaaaacaaaacaaacacgccaaaaacctttattctctttattatgaatcaaatttttcctctcagataattgttttatttatttatttttattattattgttattatgtccagtctcactctgtcgcctaagctggcatgat

SEQ ID NO: 8 - DNA, 1821 bp, Homo sapiens
  misc_feature: sequence of STAR8
gagatcacctcgaagagagtctaacgtccgtaggaacgctctcgggttcacaaggattgaccgaaccccaggatacgtcgctctccatctgaggcttgctccaaatggccctccactattccaggcacgtgggtgtctcccctaactctccctgctctcctgagcccatgctgcctatcacccatcggtgcaggtcctttctgaagagctcgggtggattctctccatcccacttcctttcccaagaaagaagccaccgttccaagacacccaatgggacattccccttccacctccttctccaaagttgcccaggtgttcatcacaggttagggagagaagcccccaggtttcagttacaaggcataggacgctggcatgaacacacacacacacacacacacacacacacacacacacacacgactcgaagaggtagccacaagggtcattaaacacttgacgactgttttccaaaaacgtggatgcagttcatccacgccaaagccaagggtgcaaagcaaacacggaatggtggagagattccagaggctcaccaaaccctctcaggaatattttcctgaccctgggggcagaggttggaaacattgaggacatttcttgggacacacggagaagctgaccgaccaggcattttcctttccactgcaaatgacctatggcgggggcatttcactttcccctgcaaatcacctatggcgaggtacctccccaagcccccacccccacttccgcgaatcggcatggctcggcctctatccgggtgtcactccaggtaggcttctcaacgctctcggctcaaagaaggacaatcacaggtccaagcccaaagcccacacctcttccttttgttatacccacagaagttagagaaaacgccacactttgagacaaattaagagtcctttatttaagccggcggccaaagagatggctaacgctcaaaattctctgggccccgaggaaggggcttgactaacttctataccttggtttaggaaggggaggggaactcaaatgcggtaattctacagaagtaaaaacatgcaggaatcaaaagaagcaaatggttatagagagataaacagttttaaaaggcaaatggttacaaaaggcaacggtaccaggtgcggggctctaaatccttcatgacacttagatataggtgctatgctggacacgaactcaaggctttatgttgttatctcttcgagaaaaatcctgggaacttcatgcactgtttgtgccagtatcttatcagttgattgggctcccttgaaatgctgagtatctgcttacacaggtcaactccttgcggaagggggttgggtaaggagcccttcgtgtctcgtaaattaaggggtcgattggagtttgtccagcattcccagctacagagagccttatttacatgagaagcaaggctaggtgattaaagagaccaacagggaagattcaaagtagcgacttagagtaaaaacaaggttaggcatttcactttcccagagaacgcgcaaacattcaatgggagagaggtcccgagtcgtcaaagtcccagatgtggcgagcccccgggaggaaaaaccgtgtcttccttaggatgcccggaacaagagctaggcttccggagctaggcagccatctatgtccgtgagccggcgggagggagaccgccgggaggcgaagtggggcggggccatccttctttctgctctgctgctgccggggagctcctggctggcgtccaagcggcaggaggccgccgtcctgcagggcgccgtagagtttgcggtgcagagt

SEQ ID NO: 9 - DNA, 1929 bp, Homo sapiens
  misc_feature: sequence of STAR9
cacttcctgggagtggagcagaggctctgcgtggagcatccatgtgcagtactcttaggtacggaagggattgggctaaaccatggatgggagctgggaagggaagggaccaacttcaggccccactgggacactggagctgccaccctttagagccctcctaaccctacaccagaggctgagggggacctcagacatcacacacatgctttcccatgttttcagaaatctggaaacgtagaacttcaggggtgagagtgcctagatattgaatacaaggctagattgggcttctgtaatatcccaaaggaccctccagctttttcaccagcacctaatgcccatcagataccaaagacacagcttaggagaggttcaccctgaagctgaggaggaggcagccggattagagttgactgagcaaggatgactgccttctccacctgacgatttcagctgctgcccttttcttttcctgggaatgcctgtcgccatggccttctgtgtccacaggagagtttgacccagatactcatggaccaggcaaaggtgctgttcctcccagcccagggcccaccatgaagcatgcctgggagcctggtaaggacccagccactcctgggctgttgacattggcttctcttgcccagcattgtagccacgccactgcattgtactgtgagataagtcaaggtgggctcaccaggacctgcactaaattgtgaaattcagctccaaagaactttggaaattacccatgcatttaagcaaaatgaatgatacctgagcaaaccctttcacattggcacaagttacaatcctgtctcatcctcttgattacaaattccatccaggcaagagctgtatcaccctgaggtctccccattcatgttttggtcaataatatttagtttccttttgaaaatagatttttgtgttactccattatgatgggcagaggccagatgcttatattctatttaaatgactatgtttttctatctgtaactgggtttgtgttcaggtggtaaatgctttttttttgcagtcagaagattcctggaaggcgaccagaaattagctggccgctgtcagacctgaagttacttctaaagggcctttagaaatgaattcttttttatgccttctctgaattctgagaagtaggcttgacttcccctaagtgtggagttgggagtcaactcttctgaaaagaaagtttcagagcattttccaaagccatggtcagctgtgggaagggaagacgatggatagtacagttgccggaaaacactgatggaggcggatgctccagctcagccaaagacctttgttctgcccaccccagaaatgccccttcctcaatcgcagaaacgttgccccatggctcctgatactcagaatgcagcctctgaccaggaccatctgcatcctccaggagctcgtaagaaatgcagcatcgtgggacctgctggcacctggtgaacccaaacctgcagggctcctgggtgtgcttggggcggctgcaggggaagagggagtcagcagcctcctcctgaccttcccgggggctgcttttctgaggggccagaatgcaccggttgaccttgttgcatcactggcccatgactggctgctttggtcaggtgtaaaaaggtgtttccagagggtctgctcctctcactatcggaccaggtttccatggagagctcagcctcccagcaaggatagagaacttcaaatggctcaaagaactgagaggccacacatgtgtgacctgaatagtctctgctgcaaaacaaagggtttcttaatgtaaaacgttctcttcctcacagaggggttcccagctgctagtgggcatgttgcaggcatttcctgggctgcatcaggttgtcataagccagaggatcatttttgggggctcat

SEQ ID NO: 10 - DNA, 1167 bp, Homo sapiens
  misc_feature: sequence of STAR10
aggtcaggagttcaagaccagcctggccaacatggtgaaaccctgtccctacaaaaaatacaaaaattagccgggcgtggtggggggcgcctataatcccagctactcaggatgctgagacaggagaattgtttgaacccgggaggtggaggttgcagtgaactgagatcgcgccactgcactccagcctggtgacagagagagactccgtctcaacaacagacaaacaaacaaacaaacaacaacaaaaatgtttactgacagctttattgagataaaattcacatgccataaaggtcaccttctacagtatacaattcagtggatttagtatgttcacaaagttgtacgttgttcaccatctactccagaacatttacatcacccctaaaagaagctctttagcagtcacttctcattctccccagcccctgccaaccacgaatctactntctgtctctattctgaatatttcatataaaggagtcctatcatatgggccttttacgtctaccttctttcacttagcatcatgtttttaagattcatccacagtgtagcacgtgtcagttaattcatttcatcttatggctggataatgctctattgtatgcatatccctcactttgcttatccattcatcaactgattgacatttgggttatttctactttttgactattatgagtaatgctgctatgaacattcctgtaccaatcgttacgtggacatatgctttcaattctcctgagtatgtaactagggttggagttgctgggtcatatgttaactcagtgtttcatttttttgaagaactaccaaatggttttccaaagtggatgcaacactttacattcccaccagcaagatatgaaggttccaatgtctctacatttttgccaacacttgtgattttcttttatttatttatttatttatttatttttgagatggagtctcactctgtcacccaggctggagtgcagtggcacaatttcagctcactgcaatctccacctctcgggctcaagcgatactcctgcctcaacctcccgagtaactgggattacaggcgcccaccaccacaccaagctaattttttgtatttttagtagagacggggtttcatcatgtcggccaggntgtactcgaactctgacctcaagt

SEQ ID NO: 11 - DNA, 1377 bp, Homo sapiens
  misc_feature: sequence of STAR11
aggatcacttgagcccaggagttcaagaccagcctgggcaacatagcgagaacatgtctcaaaaaggaaaaaaatgggggaaaaaaccctcccagggacagatatccacagccagtcttgataagctccatcattttaaagtgcaaggcggtgcctcccatgtggatgattatttaatcctcttgtactttgtttagtcctttgtggaaatgcccatcttataaattaatagaattctagaatctaattaaaatggttcaactctacattttactttaggataatatcaggaccatcacagaatgtctgagatgtggatttaccctatctgtagctcacttcttcaaccattcttttagcaaggctagttatcttcagtgacaaccccttgctgccctctactatctcctccctcagatggactactctgattaagcttgagctagaataagcatgttatcccgggatttcatatggaatattttatacatgagtgagccattatgagttgtttgaaaatttattatgttgagggagggtaaccgctgtaacaaccatcaccaaatctaatcgactgaatacatttgacgtttatttcttgttcacctgacagttcagtgttacctaaatttacatgaagacccagaggcccacgctccttcattttgggctccaccgacctccaaggtttcagggccctctgccccgccttctgcacccacaggggaagagagtggaggatgcacacgcccaggcctggaagtgacgcatgtggcttccccgtccacagacttcacccacagtccattggccttcttaagtcatggactcctgctgagctgccagggtgcatgggaaatccatgtgactgtgtgccctggaggaaggggagcgtttcggtgagcacacaggagtctttgccactagacgctgatgaggattccccacaggcgatgaagcatggagactcatcttgtaacaaacagatgagttgttgacatctcttaagtttactttgtgtgcagtttttattcagataggaaaggctgttaaaatcttaacacctaactggaagaagggttttagagaagtgtggttttcagtaagccagttctttccacaatccaagaaacgaaataaatttccagcatggagcagttggcaggtaaggtttttgttgtggtctcgcccaggcttgagtgtaaccggtgtggtcatagctcactacattctcaaactcctggccttaagtcatcctcctgcctcagcctcccaaaggcaagtaaggttaagaataggggaaaggtgaagtttcacagcttttctagaattctttttattcaagggactctcagatcatcaaacccacccagaatc

SEQ ID NO: 12 - DNA, 1051 bp, Homo sapiens
  misc_feature: sequence of STAR12
atcctgcttctgggaagagagtggcctcccttgtgcaggtgactttggcaggaccagcagaaacccaggtttcctgtcaggaggaagtgctcagcttatctctgtgaagggtcgtgataaggcacgaggaggcaggggcttgccaggatgttgcctttctgtgccatatgggacatctcagcttacgttgttaagaaatatttggcaagaagatgcacacagaatttctgtaacgaataggatggagttttaagggttactacgaaaaaaagaaaactactggagaagagggaagccaaacaccaccaagtttgaaatcgattttattggacgaatgtctcactttaaatttaaatggagtccaacttccttttctcacccagacgtcgagaaggtggcattcaaaatgtttacacttgtttcatctgcctttttgctaagtcctggtcccctacctcctttccctcacttcacatttgtcgtttcatcgcacacatatgctcatctttatatttacatatatataatttttatatatggcttgtgaaatatgccagacgagggatgaaatagtcctgaaaacagctggaaaattatgcaacagtggggagattgggcacatgtacattctgtactgcaaagttgcacaacagaccaagtttgttataagtgaggctgggtggtttttattttttctctaggacaacagcttgcctggtggagtaggcctcctgcagaaggcattttcttaggagcctcaacttccccaagaagaggagagggcgagactggagttgtgctggcagcacagagacaagggggcacggcaggactgcagcctgcagaggggctggagaagcggaggctggcacccagtggccagcgaggcccaggtccaagtccagcgaggtcgaggtctagagtacagcaaggccaaggtccaaggtcagtgagtctaaggtccatggtcagtgaggctgagacccagggtccaatgaggccaaggtccagagtccagtaaggccgagatccagggtccagggaggtcaag

SEQ ID NO: 13 - DNA, 1291 bp, Homo sapiens
  misc_feature: sequence of STAR13
agccactgaggtcctaactgcagccaaggggccgttctgcacatgtcgctcaccctctgtgctctgttccccacagagcaaacgcacatggcaacgttggtccgctcagccactggttctgtggtggaacggtggatgtctgcactgtgacatcagctgagtaagtaacaacgactgaggatgccgctgacccagggctggggaaggggactcccagctcagacaggcttggctgtggtttgctttgggaggagagtgaacatcacagggaatggctcatgtcagccccaggagggtgggctggcccctggtccccgggctccttctggccctgcaggcgatagagagcctcaacctgctgccgcttctccttggcccgggtgatggccgtctggaagagcctgcagtagaggtgcacagccagcggagagtcgtcattgccgggtacagggtaggtgatgaggcaggggttgcagttggtgtccacgatgcccactgtggggatgttcatcttggctgcgtctctcacggccacgtgtggctcaaagatgttgttgagcgtgtgcaggaagatgatgaggtccggcaggcggaccgtggggccaaagaggaggcgcgcgttggtcagcatgccgcccctgaagtagcgagtgtgggcgtactcgccacagtcacgggccatgttctcaatcaggtacgagaactgccggttgcggcttataaacaagatgatgcccttgcggtaggccatgtgggcggtgaagttcaaggccagctggaggtgcgtggctgtctgttccaggtcgatgatgtcgtggtccaggcggctcccaaagatgtacggctccataaacctgccagagaccccaccaaggcaagggggatgagagttcacggggccatctccactggctccttgcaggaacacagacgcccaccagggactcccgggctcctctgtgggggcactatgggctgggaagcacaatttgcaacgctccccgtgtgcatggacagcagtgcagacccatccaggccacccctctgcatgcctcgtctcgtggcttaacccctcctaccctctacctcttcccgaaggaatcctaatagaactgaccccatatggatgtgtggacatccaacatgacgccaaaaggacattctgccccgtgcagctcacagggcagccgcctccgtcactgtcctcttcccgaggctttgcggatgaggcccctctggggttggacttagcggggtgctctgggccaaaagcattaagggatcagggcag

SEQ ID NO: 14 - DNA, 711 bp, Homo sapiens
  misc_feature: sequence of STAR14
ccctggaccagggtccgtggtcttggtgggcactggcttcttcttgctgggtgttttcctgtgggtctctggcaaggcactttttgtggcgctgcttgtgctgtgtgcgggaggggcaggtgctctttcctcttggagctggaccctctggggcgggtccccgtcggcctccttgtgtgttttctgcacctggtacagctggatggcctcctcaatgccgtcgtcgctgctggagtcggacgcctcgggcgcctgtacggcgctcgtgactcgctttcccctccttgcggtgctggcgttccttttaatcccacttttattctgtactgcttctgaagggcggtgggggttgctggctttgtgctgccctccttctcctgcgtggtcgtggtcgtgaccttggacctgaggcttctgggctgcacgtttgtctttgctaaccgggggaggtctgcagaaggcgaactccttctggacgcccatcaggccctgccggtgcaccacctttgtagccggctcttggtgggatttcgagagtgacttcgccgaattttcatgtgtgtctggtttcttctccactgacccatcacatttttgggtctcatgctgtcttttctcattcagaaactgttctatttctgccctgatgctctgctcaaaggagtctgctctgctcatgctgactggggaggcagagccctggtccttgct

SEQ ID NO: 15 - DNA, 1876 bp, Homo sapiens
  misc_feature: sequence of STAR15
gagtccaagatcaaggtgccagcatcttgtgagggccttcttgttacgtcactccctagcgaaagggcaaagagagggtgagcaagagaaaggggggctgaactcgtccttgtagaagaggcccattcccgagacaatggcattcatccattcactccaccctcatggcctcaccacctctcatgaggctccacctcccagccctggtttgttggggattaaatttccaacacatgccttttgggggacatgttaaaattatagcaccccaaatgttacactatcttttgatgagcggtagttctgattttaagtctagctggcctactttttcttgcacgtgggatgctttctgcctgttccagggcaggcagctcttctctgtccctctgctggccccacctcatcctctgttgtcctcttccctccttctgtgccctggggtcctggtgggggtgtgactgtcaactgcgttgggctaacttttttccctgctggtggcccgtaatgaaagaaagcttcttgctcccaagttccttaaatccaagctcatagacaacgcggtctcacagcaggcctggggccagcctcacgtgagccccttccctggtgtagtcactggcatgggggaatgggatttcctgttgccctactgtgtggctgaggtgggggttgcttcctggagccaggccttgtggaagggcagtgcccactgcagtggatgctgggccctgaatctgaccccagtgttcattggctctgtgagacccagtgagggcagggagggaagtggagctggggtgagaagtagaggccctgcagggcccacgtgccagccaccaggcctcagactaggctcagatgacggagagctgcacacctgcccaacccaggccctgcagtgcccacatgccagccgctggggcccagacttgctccagagggcggagagctttacaccggcccaacccaggccatggctccaaatgcgtgacagttttgctgttgcttcttttagtcattgtcaagttgatgcttgttttgcagaggaccaaggctttatgaacctattaccctgtgtgaagagtttcaccaggttatggaaatttctttaaaaccataccacagttttttcattattcatgtatatttttaaaaataattactgcactcagtagaataacatgaaaatgttgcctgttagcccttttccagtttgccccgagaatactgggggcacttgtggctgcaatgtttatcctgcggcagctttgccatgaagtatctcacttttattattatttttgcattgctcgagtatattgactttggaaacaaaagacatcattctatttatagcattatgtttttagtagtggtatttccatatacaagatacagtaattttccgtcaatgaaaatgtcaaattctagaaaatgtaacattcctatgcgtggtgttaacatcgttctctaacagttgttggccgaagattcgtttgatgaatccgatttttccaaaatagccgattctgatgattcagacgattctgatgttctgtttagaaataattccaagaacagtttttacattttattttcacattgaaaatcagtcagatttgcttcagcctcaaagagcacgtttatgtaaaattaaatgagtgctggcagccagctgcgctttgtttttctaaatgggaaaagggttaaatttcactcagcttttaaatgacagcgcacagcctgtgtcatagagggttggaggagatgactttaactgcctgtggttaggatccctttcccccaggaatgtctgggagcccactgccgggtttgctgtccgtctcgtttggactcagttctgcatgtactg

SEQ ID NO: 16 - DNA, 1282 bp, Homo sapiens
  misc_feature: sequence of STAR16
cgcccacctcggctttccaaagtgctgggattacaggcatgagtcactgcgcccatcctgattccaagtctttagataataacttaactttttcgaccaattgccaatcaggcaatctttgaatctgcctatgacctaggacatccctctccctacaagttgccccgcgtttccagaccaaaccaatgtacatcttacatgtattgattgaagttttacatctccctaaaacatataaaaccaagctatagtctgaccacctcaggcacgtgttctcaggacctccctggggctatggcatgggtcctggtcctcagatttggctcagaataaatctcttcaaatattttccagaattttactcttttcatcaccattacctatcacccataagtcagagttttccacaaccccttcctcagattcagtaatttgctagaatggccaccaaactcaggaaagtattttacttacaattaccaatttattatgaagaactcaaatcaggaatagccaaatggaagaggcatagggaaaggtatggaggaaggggcacaaagcttccatgccctgtgtgcacaccaccctctcagcatcttcatgtgttcaccaactcagaagctcttcaaactttgtcatttaggggtttttatggcagttccactatgtaggcatggttgataaatcactggtcatcggtgatagaactctgtctccagctcctctctctctcctccccagaagtcctgaggtggggctgaaagtttcacaaggttagttgctctgacaaccagcccctatcctgaagctattgaggggtcccccaaaagttaccttagtatggttggaagaggcttattatgaataacaaaagatgctcctatttttaccactagggagcatatccaagtcttgcgggaacaaagcatgttactggtagcaaattcatacaggtagatagcaatctcaattcttgccttctcagaagaaagaatttgaccaagggggcataaggcagagtgagggaccaagataagttttagagcaggagtgaaagtttattaaaaagttttaggcaggaatgaaagaaagtaaagtacatttggaagagggccaagtgggcgacatgagagagtcaaacaccatgccctgtttgatgtttggcttggggtcttatatgatgacatgcttctgagggttgcatccttctcccctgattcttcccttggggtgggctgtccgcatgcacaatggcctgccagcagtagggaggggccgcatg

SEQ ID NO: 17 - DNA, 793 bp, Homo sapiens
  misc_feature: sequence of STAR17
atccgaggggaggaggagaagaggaaggcgagcagggcgccggagcccgaggtgtctgcgagaactgttttaaatggttggcttgaaaatgtcactagtgctaagtggcttttcggattgtcttatttattactttgtcaggtttccttaaggagagggtgtgttgggggtgggggaggaggtggactggggaaacctctgcgtttctcctcctcggctgcacagggtgagtaggaaacgcctcgctgccacttaacaatccctctattagtaaatctacgcggagactctatgggaagccgagaaccagtgtcttcttccagggcagaagtcacctgttgggaacggcccccgggtccccctgctgggctttccggctcttctaggcggcctgatttctcctcagccctccacccagcgtccctcagggacttttcacacctccccacccccatttccactacagtctcccagggcacagcacttcattgacagccacacgagccttctcgttctcttctcctctgttccttctctttctcttctcctctgttccttctctttctctgtcataatttccttggtgctttcgccaccttaaacaaaaaagagaaaaaaataaaataaaaaaaacccattctgagccaaagtattttaagatgaatccaagaaagcgacccacatagccctccccacccacggagtgcgccaagacgcacccaggctccatcacagggccgagagcagcgccactctggtcgtacttttgggtcaagagatcttgcaaaagagg

SEQ ID NO: 18 - DNA, 492 bp, Homo sapiens
  misc_feature: sequence of STAR18
atctttttgctctctaaatgtattgatgggttgtgttttttttcccacctgctaataaatattacattgcaacattcttccctcaacttcaaaactgctgaactgaaacaatatgcataaaagaaaatcctttgcagaagaaaaaaagctattttctcccactgattttgaatggcacttgcggatgcagttcgcaaatcctattgcctattccctcatgaacattgtgaaatgaaacctttggacagtctgccgcattgcgcatgagactgcctgcgcaaggcaagggtatggttcccaaagcacccagtggtaaatcctaacttattattcccttaaaattccaatgtaacaacgtgggccataaaagagtttctgaacaaaacatgtcatctttgtggaaaggtgtttttcgtaattaatgatggaatcatgctcatttcaaaatggaggtccacgatttgtggccagctgatgcctgcaaattatcct

SEQ ID NO: 19 - DNA, 1840 bp, Homo sapiens
  misc_feature: sequence of STAR19
tcacttcctgatattttacattcaaggctagctttatgcatatgcaacctgtgcagttgcacagggctttgtgttcagaaagactagctcttggtttaatactctgttgttgccatcttgagattcattataatataatttttgaatttgtgttttgaacgtgatgtccaatgggacaatggaacattcacataacagaggagacaggtcaggtggcagcctcaattccttgccacccttttcacatacagcattggcaatgccccatgagcacaaaatttgggggaaccatgatgctaagactcaaagcacatataaacatgttacctctgtgactaaaagaagtggaggtgctgacagcccccagaggccacagtttatgttcaaaccaaaacttgcttagggtgcagaaagaaggcaatggcagggtctaagaaacagcccatcatatccttgtttattcatgttacgtccctgcatgaactaatcacttacactgaaaatattgacagaggaggaaatggaaagatagggcaacccatagttctttttccttttagtctttccttatcagtaaaccaaagatagtattggtaaaatgtgtgtgagttaattaatgagttagttttaggcagtgtttccactgttggggtaagaacaaaatatataggcttgtattgagctattaaatgtaaattgtggaatgtcagtgattccaagtatgaattaaatatccttgtatttgcatttaaaattggcactgaacaacaaagattaacagtaaaattaataatgtaaaagtttaatttttacttagaatgacattaaatagcaaataaaagcaccatgataaatcaagagagagactgtggaaagaaggaaaacgtttttattttagtatatttaatgggactttcttcctgatgttttgttttgttttgagagagagggatgtgggggcagggaggtctcattttgttgcccaggctggacttgaactcctgggctccagctatcctgccttagcttcttgagtagctgggactacaggcacacaccacagtgtctgacattttctggattttttttttttttttattttttttgtgagacaggttctggctctgttactcaggttgcagtgcagtggcatgatagcggctcactgcagcctcaacctcctcagcttaagctactctcccacttcagcctcctgagtagccaggactacagttgtgtgccaccacacctgtggctaatttttgtagagatggggtctctccacgttgccgaggctggtctccaactcctggtctcaagcgaacctcctgacttggcctcccgaagtgctgggattacaggcttgagccactgcatccagcctgtcctctgtgttaaacctactccaatttgtctttcatctctacataaacggctcttttcaaagttcccatagacctcactgttgctaatctaataataaattatctgccttttcttacatggttcatcagtagcagcattagattgggctgctcaattcttcttggtatattttcttcatttggcttctggggcatcacactctctttgagttactcattcctcattgatagcttcttcctagtcttctttactggttcttcctcttctccctgactccttaatattgtttttctccccaggctttagttcttagtcctcttctgttatctatttacacccaattctttcagagtctcatccagagtcatgaacttaaacctgtttctgtgcagataattcacattattatatctccagcccagactctcccgcaaactgcagactgatcctactg

SEQ ID NO: 20 - DNA, 780 bp, Homo sapiens
  misc_feature: sequence of STAR20
gatctcaagtttcaatatcatgttttggcaaaacattcgatgctcccacatccttacctaaagctaccagaaaggctttgggaactgtcaacagagctacagaaaagtcagtaaagaccaatggacccctcaaacaaaaacagccaagcttttctgccaaaaagatgactgagaagactgttaaagcaaaaaactctgttcctgcctcagatgatggctatccagaaatagaaaaattatttcccttcaatcctctaggcttcgagagttttgacctgcctgaagagcaccagattgcacatctccccttgagtgaagtgcctctcatgatacttgatgaggagagagagcttgaaaagctgtttcagctgggccccccttcacctttgaagatgccctctccaccatggaaatccaatctgttgcagtctcctttaagcattctgttgaccctggatgttgaattgccacctgtttgctctgacatagatatttaaatttcttagtgctttagagtttgtgtatatttctattaataaagcattatttgtttaacagaaaaaaagatatatacttaaatcctaaaataaaataaccattaaaaggaaaaacaggagttataactaataagggaacaaaggacataaaatgggataataatgcttaatccaaaataaagcagaaaatgaagaaaaatgaaatgaagaacagataaatagaaaacaaatagcaatatgaaagacaaacttgaccgggtgtggtggctgatgcctgtaatcc

SEQ ID NO: 21 - DNA, 607 bp, Homo sapiens
  misc_feature: sequence of STAR21
gatcaataatttgtaatagtcagtgaatacaaaggggtatatactaaatgctacagaaattccattcctgggtataaatcctagacatatttatgcatatgtacaccaagatatatctgcaagaatgttcacagcaaatctctttgtagtagcaaaaggccaaaaggtctatcaacaagaaaattaatacattgtggcacataatggcatccttatgccaataaaaatggatgaaattatagttaggttcaaaaggcaagcctccagataatttatatcatataattccatgtacaacattcaacaacaagcaaaactaaacatatacaaatgtcagggaaaatgatgaacaaggttagaaaatgattaatataaaaatactgcacagtgataacatttaatgagaaaaaaagaaggaagggcttagggagggacctacagggaactccaaagttcatggtaagtactaaatacataatcaaagcactcaaaatagaaaatattttagtaatgttttagctagttaatatcttacttaaaacaaggtctaggccaggcacggtggctcacacctgtaatcccagcactttgggaggctgaggcgggt

SEQ ID NO: 22 - DNA, 1380 bp, Homo sapiens
  misc_feature: sequence of STAR22
cccttgtgatccacccgccttggcctcccaaagtgctgggattacaggcgtgagtcactacgcccggccaccctccctgtatattatttctaagtatactattatgttaaaaaaagtttaaaaatattgatttaatgaattcccagaaactaggattttacatgtcacgttttcttattataaaaataaaaatcaacaataaatatatggtaaaagtaaaaagaaaaacaaaaacaaaaagtgaaaaaaataaacaacactcctgtcaaaaaacaacagttgtgataaaacttaagtgcctgaaaatttagaaacatccttctaaagaagttctgaataaaataaggaataaaataatcacatagttttggtcattggttctgtttatgtgatggattatgtttattgatttgtgtatgttgaacttatctcaatagatgcagacaaggccttgataaaagtttttaacaccttttcatgttgaaaactctcaatagactaggtattgatgaaacatatctcaaaataatagaagctatttatgataaacccatagccaatatcatactgagtgggcaaaagctggaagcattccctttgaaaactggcacaagacaaggatgccctctctcaccactcctattaaatgtagtattggaagttctggccagagcaatcaggcaggagaaagaaaaggtattaaaataggaagagaggaagtcaaattgtctctgtttgcagtaaacatgattgtatatttagaaaaccccattgtctcatcctaaaaactccttaagctgataaacaacttcagcaaagtctcaggatacaaaatcaatgtgcaaaaatcacaagcattcctatacaccgataatagacagcagagagccaaatcatgagtgaagtcccattcacaattgcttcaaagaaaataaaatacttaggaatacaactttcacgggacatgaaggacattttcaaggacaactaaaaaccactgctcaaggaaatgagagaggacacaaagaaatggaaaaacattccatgctcatggaagaatcaatatcatgaaaatggccatactgcccaaagtaatttatagattcaatgctaaccccatcaagccaccattgactttcttcacagaactagaaaaaaactattttaaaactcatatgtagtcaaaaagagtcggtatagccaagacaatcctaagcataaagaacaaagctggatgcatcacgctgacttcaaaccatactacaaggctacagtaaccaaaacagcatggtactggtaccaaaacagatagatagaccgatagaacagaacagaggcctcggaaataacaccacacatctacaaccctttgatcttcaa

SEQ ID NO: 23 - DNA, 1246 bp, Homo sapiens
  misc_feature: sequence of STAR23
atcccctcatccttcagggcagctgagcagggcctcgagcagctgggggagcctcacttaatgctcctgggagggcagccagggagcatggggtctgcaggcatggtccagggtcctgcaggcggcacgcaccatgtgcagccgcccccacctgttgctctgcctccgccacctggccatgggcttcagcagccagccacaaagtctgcagctgctgtacatggacaagaagcccacaagcagctagaggaccttgtgttccacgtgcccagggagcatggcccacagcccaaagaccagtcaggagcaggcaggggcttctggcaggcccagctctacctctgtcttcacacagatgggagatttctgttgtgattttgagtgatgtgcccctttggtgacatccaagatagttgctgaagcaccgctctaacaatgtgtgtgtattctgaaaacgagaacttctttattctgaaataattgatgcaaaataaattagtttggatttgaaattctattcatgtaggcatgcacacaaaagtccaacattgcatatgacacaaagaaaagaaaaagcttgcattccttaaatacaaatatctgttaactatatttgcaaatatatttgaatacacttctattatgttacatataatattatatgtatatgtatatataatatacatatatatgttacatataatatacttctattatgttacatataatatttatctataagtaaatacataaatataaagatttgagtagctgtagaacattgtcttatgtgttatcagctactactacaaaaatatctcttccacttatgccagtttgccatataaatatgatcttctcattgatggcccagggcaagagtgcagtgggtacttattctctgtgaggagggaggagaaaagggaacaaggagaaagtcacaaagggaaaactctggtgttgccaaaatgtcaagtttcacatattccgagacggaaaatgacatgtcccacagaaggaccctgcccagctaatgtgtcacagatatctcaggaagcttaaatgatttttttaaaagaaaagagatggcattgtcacttgtttcttgtagctgaggctgtgggatgatgcagatttctggaaggcaaagagctcctgctttttccacaccgagggactttcaggaatgaggccagggtgctgagcactacaccaggaaatccctggagagtgtttttcttactta

SEQ ID NO: 24 - DNA, 939 bp, Homo sapiens
  misc_feature: sequence of STAR24
acgaggtcacgagttcgagaccagcctggccaagatggtgaagccctgtctctactaaaaatacaacaagtagccgggcgcggtgacgggcgcctgtaatcccagctactcaggaggctgaagcaggagaatctctagaacccaggaggcggaggtgcagtgagctgagactgccccgctgcactctagcctgggcaacacagcaagactctgtctcaaataaataaataaataaataaataaataaataaataaataaatagaaagggagagttggaagtagatgaaagagaagaaaagaaatcctagatttcctatctgaaggcaccatgaagatgaaggccacctcttctgggccaggtcctcccgttgcaggtgaaccgagttctggcctccattggagaccaaaggagatgactttggcctggctcctagtgaggaagccatgcctagtcctgttctgtttgggcttgatcctgtatcacttgattgtctctcctggactttccatggattccagggatgcaactgagaagtttatttttaatgcacttacttgaagtaagagttattttaaaacattttagcaaaggaaatgaattctgacaggttttgcactgaagacattcacatgtgaggaaaacaggaaaaccactatgctagaaaaagcaaatgctgttgagattgtctcacaaacacaaattgcgtgccagcaggtaggtttgagcctcaggttgggcacattttaccttaagcgcactgttggtggaacttaaggtgactgtaggacttatatatacatacatacatataatatatatacatatttatgtgtatatacacacacacacacacacacacacacagggtcttgctatcttgcccagggtggtctccaactctgggtctcaagcgatcctctgcctccccttcccaaag

SEQ ID NO: 25 - DNA, 1067 bp, Homo sapiens
  misc_feature: sequence of STAR25
cagcccctcttgtgtttttctttatttctcgtacacacacgcagttttaagggtgatgtgtgtataattaaaaggacccttggcccatactttcctaattctttagggactgggattgggtttgactgaaatatgttttggtggggatgggacggtggacttccattctccctaaactggagttttggtcggtaatcaaaactaaaagaaacctctgggagactggaaacctgattggagcactgaggaacaagggaatgaaaaggcagactctctgaacgtttgatgaaatggactcttgtgaaaattaacagtgaatattcactgttgcactgtacgaagtctctgaaatgtaattaaaagtttttattgagcccccgagctttggcttgcgcgtatttttccggtcgcggacatcccaccgcgcagagcctcgcctccccgctgccctcagcctccgatgacttccccgcccccgccctgctcggtgacagacgttctactgcttccaatcggaggcacccttcgcgggagcggccaatcgggagctccggcaggcggggaggccgggccagttagatttggaggttcaacttcaacatggccgaagcaagtagcgccaatctaggcagcggctgtgaggaaaaaaggcatgaggggtcgtcttcggaatctgtgccacccggcactaccatttcgagggtgaagctcctcgacaccatggtggacacttttcttcagaagctggtcgccgccggcaggtaaagtggacgcagccgcggtgggagtgtttgttggcaccgaagctcaaatcccgcgaggtcaggacggccgcaggctggcgcgcggtgacgtgggtccgcgttgggggcggggcagtcggacgaggcgacccagtcaaatcctgagccttaggagtcagggtattcacgcactgataacctgtagcggaccgggatagctagctactccttcctacaggaagccccgttttcactaaaatttcaggtggttgggaggaaagatagagcctttgcaaattagagcagggttttttatttttttat

SEQ ID NO: 26 - DNA, 540 bp, Homo sapiens
  misc_feature: sequence of STAR26
ccccctgacaagccccagtgtgtgatgttccccactctgtgtccatgcattctcattgttcaactcccatctgtgagtgagaacatgcagtgtttggttttctgtccttgagatagtttgctgagaatgatggtttccagcttcatccatgtccttgcaaaggaagtgaacttatccttttttatggcttcatagtattccatggcacatatgtgccacatttttttaatccagtctatcattgatggacatttgggttggttccaagtctttgctattgtgaatagcaccacaattaacatatgtgtgcatgtatacatctttatagtagcatgatttataatccttcgggtatataccctgtaatgggatcgctgggtcaaatggtatttctagttctagatccttgaggaatcaccacactgctttccacaatggttgaactaatttacgctcccaccagcagtgtaaaagcattcctatttctccacgtcctctccagtatctgttgtttcctgactttttaatgatcatcattct

SEQ ID NO: 27 - DNA, 1520 bp, Homo sapiens
  misc_feature: sequence of STAR27
cttggccctcacaaagcctgtggccagggaacaattagcgagctgcttattttgctttgtatccccaatgctgggcataatgcctgccattatgagtaatgccggtagaagtatgtgttcaaggaccaaagttgataaataccaaagaatccagagaagggagagaacattgagtagaggatagtgacagaagagatgggaacttctgacaagagttgtgaagatgtactaggcagggggaacagcttaaggagagtcacacaggaccgagctcttgtcaagccggctgccatggaggctgggtggggccatggtagctttcccttccttctcaggttcagagtgtcagccttgaacttctaattcccagaggcatttattcaatgttttcttctaggggcatacctgccctgctgtggaagactttcttccctgtgggtcgccccagtccccagatgagacggtttgggtcagggccaggtgcaccgttgggtgtgtgcttatgtctgatgacagttagttactcagtcattagtcattgagggaggtgtggtaaagatggagatgctgggtcacatccctagagaggtgttccagtatgggcacatgggagggctggaaggataggttactgctagacgtagagaagccacatcctttaacaccctggcttttcccactgccaagatccagaaagtccttgtggtttcgctgctttctcctttttttttttttttttttttctgagatggagtctggctctgtcgcccaggctggagtgcagtggcacgatttcggctcactgcaagttccgcctcctaggttcataccattctcccacctcagcctcccgagtagctgggactacaggcgccaccacacccagctaattttttgtatttttagtagagacggcgtttcaccatgttagccaggatggtcttgatccgcctgcctcagcctcccaaagtgctgggattacaggcgtgagccaccgcgcccggcctgctttcttctttcatgaagcattcagctggtgaaaaagctcagccaggctggtctggaactcttgacctcaagtgatctgcctgcctcagcctcccaaagtgctgagattacaggcatgagccagtccgaatgtggctttttttgttttgttttgaaacaaggtctcactgttgcccaggctgcagtgcagtggcatacctcagctccactgcagcctcgacctcctgggctcaagcaatcctcccaactgagcctccccagtagctggggctacaagcgcatgccaccacgcctggctatttttttttttttttttttttttgagaaggagtttcattcttgttgcccaggctggagtgcaatggcacagtctcagctcactgcagcctccgcctcctgggttcaagcgattctcctgcctcagcctcccgagtagctgggattataggcacctgccaccatgcctggctaatttttttgtatttttagtagggatggggtttcaccatgtt

SEQ ID NO: 28 - DNA, 961 bp, Homo sapiens
  misc_feature: sequence of STAR28
aggaggttattcctgagcaaatggccagcctagtgaactggataaatgcccatgtaagatctgtttaccctgagaagggcatttcctaactctccctataaaatgccaagtggagcaccccagatgaaatagctgatatgctttctatacaagccatctaggactggctttatcatgaccaggatattcacccactgaatatggctattacccaagttatggtaaatgctgtagttaagggggtcccttccacatggacaccccaggttataaccagaaagggttcccaatctagactccaagagagggttcttagacctcatgcaagaaagaacttggggcaagtacataaagtgaaagcaagtttattaagaaagtaaagaaacaaaaaaatggctactccataagcaaagttatttctcacttatatgattaataagagatggattattcatgagttttctgggaaaggggtgggcaattcctggaactgagggttcctcccacttttagaccatatagggtatcttcctgatattgccatggcatttgtaaactgtcatggcactgatgggagtgtcttttagcattctaatgcattataattagcatataatgagcagtgaggatgaccagaggtcacttctgttgccatattggtttcagtggggtttggttggcttttttttttttttaaccacaacctgttttttatttatttatttatttatttatttatttatattttttatttttttttagatggagtcttgctctgtcacccaggttagagtgcagtggcaccatctcggctcactgcaagctctgcctccttggttcacgccattctgctgcctcagcctcccgagtagctgggactacaggtgcctgccaccatacccggctaattttttctatttttcagtagagacggggtttcaccgtgttagccaggatggtc

SEQ ID NO: 29 - DNA, 2233 bp, Homo sapiens
  misc_feature: sequence of STAR29
agcttggacacttgctgatgccactttggatgttgaagggccgccctctcccacaccgctggccacttttaaatatgtcccctctgcccagaagggccccagaggaggggctggtgagggtgacaggagttgactgctctcacagcagggggttccggagggaccttttctccccattgggcagcatagaaggacctagaagggccccctccaagcccagctgggcgtgcagggccagcgattcgatgccttcccctgactcaggtggcgctgtcctaaaggtgtgtgtgttttctgttcgccagggggtggcggatacagtggagcatcgtgcccgaagtgtctgagcccgtggtaagtccctggagggtgcacggtctcctccgactgtctccatcacgtcaggcctcacagcctgtaggcaccgctcggggaagcctctggatgaggccatgtggtcatccccctggagtcctggcctggcctgaagaggaggggaggaggaggccagcccctccctagccccaaggcctgcgaggctgcaagcccggccccacattctagtccaggcttggctgtgcaagaagcagattgcctggccctggccaggcttcccagctaggatgtggtatggcaggggtgggggacattgaggggctgctgtagcccccacaacctccccaggtagggtggtgaacagtaggctggacaagtggacctgttcccatctgagattcaagagcccacctctcggaggttgcagtgagccgagatccctccactgcactccagcctgggcaacagagcaagactctgtctcaaaaaaacagaacaacgacaacaaaaaacccacctctggcccactgcctaactttgtaaataaagttttattggcacatagacacacccattcatttacatactgctgcggctgcttttgcattacccttgagtagacgacagaccacgtggccatggaagccaaaaatatttactgtctggccctttacagaagtctgctctagagggagaccccggcccatggggcaggaccactgggcgtgggcagaagggaggcctcggtgcctccacgggcctagttgggtatctcagtgcctgtttcttgcatggagcaccaggggtcagggcaagtacctggaggaggcaggctgttgcccgcccagcactgggacccaggagaccttgagaggctcttaacgaatgggagacaagcaggaccagggctcccattggctgggcctcagtttccctgcctgtaagtgagggagggcagctgtgaaggtgaactgtgaggcagagcctctgctcagccattgcaggggcggctctgccccactcctgttgtgcacccagagtgaggggcacggggtgagatgtcaccatcagcccataggggtgtcctcctggtgccaggtccccaagggatgtcccatcccccctggctgtgtggggacagcagagtccctggggctgggagggctccacactgttttgtcagtggtttttctgaactgttaaatttcagtggaaaattctctttccccttttactgaaggaacctccaaaggaagacctgactgtgtctgagaagttccagctggtgctggacgtcgcccagaaagcccaggtactgccacgggcgccggccaggggtgtgtctgcgccagccatgggcaccagccaggggtgtgtctacgccggccaggggtaggtctccgccggcctccgctgctgcctggggagggccgtgcctgacactgcaggcccggtttgtccgcggtcagctgacttgtagtcaccctgcccttggatggtcgttacagcaactctggtggttggggaaggggcctcctgattcagcctctgcggacggtgcgcgagggtggagctcccctccctccccaccgcccctggccagggttgaacgcccctgggaaggactcaggcccgggtctgctgttgctgtgagcgtggccacctctgccctagaccagagctgggccttccccggcctaggagcagccgggcaggaccacagggctccgagtgacctcagggctgcccgacctggaggccctcctggcgtcgcggtgtgactgacagcccaggagcgggggctgttgtaattgctgtttctccttcacacagaaccttttcgggaagatggctgacatcctggagaagatcaagaagtaagtcccgccccccaccc

SEQ ID NO: 30 - DNA, 1851 bp, Homo sapiens
  misc_feature: sequence of STAR30
gggtgcatttccacccaggggacacttggcaatggtgggagacattgcttgttgtcacaactgggcatgggagtgctgctgcgtctagtgggtagaggccagagatgctcctaatatcctacaaggcacagaacagccccccacaacagagaattatccagcctgaaaatgtccacagtgctgaggttgggaaaccctattctagagccaacaggctgtgaagcttgactcatggttccatcaccaatagctgcgtgaccttggtgagttccttagctgctctgtgcctcggattcatggtaggttttccttgttaggtttaaatgagtgaagttatacagagggcctgaagtctcatggtattttactagagcctcattgtgttttagttataattagaaattgggtaaggtaaggacacagaagaagccatctgatctgggggcttcacacttagaagtgacctcggagcaattgtattggggtggaaagggactaacagccaggagcagagggcacattggaattggggccagagggcacagactgccttgtccatcaggcatagcaatggacagaggaaggggaatgactagttatggctgcaaggccaagtacaggggacttatttctcatatctatctatctatctacctaccgtctatttatctatcatctatctacttatttatctatctatttatgcatgtgtaccaaccgaaagttttagtaaatgcacaaactgcgatataatgaaaatggaaattttcaaaagaagagaaatcacctgccacctgactaccttaacaaatgagtggttttcatctctccttccaggcctgtcatttttacagtgctttagtcataaaacaggtcctctattctattgttttatgtcacatgaaattgtaccataagcattttccatgatgtgactccactgtttcattttccatttttttccagaatgaagataacctcattgtttttttcctgattgtaaaaatgctctgtgctctttttttttttttttaacaatgcaggcagtaccaaaaagtatgaagaagaatgtaatagttcccatttcccatctcactctttaaggccagcattttggtgaacatccatccgaacaaatctccacgcgtttatcaatttgttgacttactccttcttttatgtaaatatgaacatgatttaactgccagtccatttggaaccttaaagtgaaggttttttattgttggggtttgctatggtctgaatatgtgtgtccccccaaaatttatgttgaatcctaacgcccaatgcgattaggaggtggggccattaggaggtgattaagtcatgaagtcatcagccctaatgaatgggatttgtggccttgaaaagggaccccagagagctgccttgccccttctgccatgtaaggacacagtgaggagctaggaagggggcctcagcagagaccaaatgtgatggtgcctcgatattggacttcccagcctccagaatgtgagaaatgaatttctgttgtttataagtcacccagtctatagtattttgttctagcagcccaaacagactaagtcagggttgttgttttaggaagtggggaatggggccatgcatgggtgtacgccagaacaaaggaagccagcaagtcctgaaagatactggaaaagggaatagtgggcacgtgcagtgtgttagtttcctgaggctgctataacaaagcaccacaggttgggtggcttaaataacagaaattcattctcccatcattctggggaccagacgtctgaaatcaagactcctatgccatgctccttctgaaggctccaggggagg

SEQ ID NO: 31 - DNA, 1701 bp, Homo sapiens
  misc_feature: sequence of STAR31
cacccgccttggccccccagagtgctgggattacaagtgtaaaccaccattcctggctagatttaattttttaaaaaataaagagaagtaggaatagttcattttagggagagccccttaactgggacaggggcaggacaggggtgaggcttcccttanttcaagctcacctcaaacccacccaggactgtgtgtcacattctccaataaaggaaaggttgctgcccccgcctgtgagtgctgcagtggagggtagagggccgtgggcagagtgcttcatggactgctcatcaagaaaggcttcatgacaatcggcccagctgctgtcatcccacattctacttccagctaggagaaggcggcttgcccacagtcacccagccggcaagtgtcacccctgggttggacccagagctatgatcctgcccaggggtccagctgagaatcaggcccacgttctaggcagaggggctcacctactgggactccagtagctgtagtgcatggaggcatcatggctgcagcagcctggacctggtctcacactggctgtccctgtgggcaggccatcctcaatgccaggtcaggcccaagcatgtatcccagacaatgacaatggggtggaatcctctcttgtcccagaagccactcctcactgttctacctgaggaaggcaggggcatggtggaatcctgaagcctgctgtgagggtctccagcgaacttgcacatggtcagccctgccttctcctccctgaactagattgagcgagagcaagaaggacattgaaccagcacccaaagaattttggggaacggcctctcatccaggtcaggctcacctcctttttaaaatttaattaattaattaattaatttttttttagagacagagtcttactgtgtggcccaggctgtagtgcagtggcacaatcatagttcactgcagcctcaaactccccacctcagcctctggattagctgagactacaggtgcaccaccaccacacccagctaatatttttatttttgtagagagagggtttcaccatcttgcccaggctggtctcaaactcctgggctcaagtgatcccgcccaggtctgaaagcccccaggctggcctcagactgtggggttttccatgcagccacccgagggcgcccccaagccagttcatctcggagtccaggcctggccctgggagacagagtgaaaccagtggtttttatgaacttaacttagagtttaaaagatttctactcgatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacagtccacctagaacagggtttgagaggcttagtcagcactgggtagcgttttgactccattctcggctttcttctttttctttccaggatttttgtgcagaaatggttcttttgttgccgtgttagtcctccttggaaggcagctcagaaggcccgtgaaatgtcgggggacaggacccccagggagggaaccccaggctacgcactttagggttcgttctccagggagggcgacctgacccccgnatccgtcggngcgcgnngnnacnaannnnttccc

SEQ ID NO: 32 - DNA, 771 bp, Homo sapiens
  misc_feature: sequence of STAR32
gatcacacagcttgtatgtgggagctaggattggaaccccagaagtctggccccaggttcatgctctcacccactgcatacaatggcctctcataaatcaatccagtataaaacattagaatctgctttaaaaccatagaattagtagcgtaagtaataaatgcagagaccatgcagtgaatggcattcctggaaaaagcccccagaaggaattttaaatcagctttcgtctaatcttgagcagctagttagcaaatatgagaatacagttgttcccagataatgctttatgtctgaccatcttaaactggcgctgtttttcaaaaacttaaaaacaaaatccatgactcttttaattataaaagtgatacatgtctacttgggaggctgaggtggtgggaggatggcttgagtttgaggctgcagtatgctactatcatgcctataaatagccgctgcattccagcttgggcaacatacccaggccctatctcaaaaaaataaaaagtaatacatctacattgaagaaaattaattttattgggtttttttgcatttttattatacacagcacacacagcacatatgaaaaaatgggtatgaactcaggcattcaactggaagaacagtactaaatcaatgtccatgtagtcagcgtgactgaggttggtttgttttttcttttttcttctcttctcttctcttttctttttttttgagacggagctttgctctttttgcccaggcttgattgcaatggcgtgatctcag

SEQ ID NO: 33 - DNA, 1368 bp, Homo sapiens
  misc_feature: sequence of STAR33
gcttttatcctccattcacagctagcctggcccccagagtacccaattctccctaaaaaacggtcatgctgtatagatgtgtgtggcttggtagtgctaaagtggccacatacagagctctgacaccaaacctcaggaccatgttcatgccttctcactgagttctggcttgttcgtgacacattatgacattatgattatgatgacttgtgagagcctcagtcttctatagcacttttagaatgctttataaaaaccatggggatgtcattatattctaacctgttagcacttctgttcgtattacccatcacatcccaacatcaattctcatatatgcaggtacctcttgtcacgcgcgtccatgtaaggagaccacaaaacaggctttgtttgagcaacaaggtttttatttcacctgggtgcaggtgggctgagtctgaaaagagagtcagtgaagggagacaggggtgggtccactttataagatttgggtaggtagtggaaaattacaatcaaagggggttgttctctggctggccagggtgggggtcacaaggtgctcagtgggagagcctttgagccaggatgagccagaaggaatttcacaaggtaatgtcatcagttaaggcagggactggccattttcacttcttttgtggtggaatgtcatcagttaaggcaggaaccggccattttcacttcttttgtgattcttcacttgcttcaggccatctggacgtataggtgcaggtcacagtcacaggggataagatggcaatggcatagcttgggctcagaggcctgacacctctgagaaactaaagattataaaaatgatggtcgcttctattgcaaatctgtgtttattgtcaagaggcacttatttgtcaattaagaacccagtggtagaatcgaatgtccgaatgtaaaacaaaatacaaaacctctgtgtgtgtgtgtgtgtgagtgtgtgtgtatgtgtgtgtgtgtgtattagagaggaaaagcctgtatttggaggtgtgattcttagattctaggttctttcctgcccaccccatatgcacccaccccacaaaagaacaaacaacaaatcccaggacatcttagcgcaacatttcagtttgcatattttacatatttacttttcttacatattaaaaaactgaaaattttatgaacacgctaagttagattttaaattaagtttgtttttacactgaaaataatttaatatttgtgaagaatactaatacattggtatatttcattttcttaaaattctgaacccctcttcccttatttccttttgacccgattggtgtattggtcatgtgactcatggatttgccttaaggcaggagg

SEQ ID NO: 34 - DNA, 755 bp, Homo sapiens
  misc_feature: sequence of STAR34
actgggcaccctcctaggcaggggaatgtgagaactgccgctgctctggggctgggcgccatgtcacagcaggagggaggacggtgttacaccacgtgggaaggactcagggtggtcagccacaaagctgctggtgatgaccaggggcttgtgtcttcactctgcagccctaacacccaggctgggttcgctaggctccatcctgggggtgcagaccctgagagtgatgccagtgggagcctcccgcccctccccttcctcgaaggcccaggggtcaaacagtgtagactcagaggcctgagggcacatgtttatttagcagacaaggtggggctccatcagcggggtggcctggggagcagctgcatgggtggcactgtggggagggtctcccagctccctcaatggtgttcgggctggtgcggcagctggcggcaccctggacagaggtggatatgagggtgatgggtggggaaatgggaggcacccgagatggggacagcagaataaagacagcagcagtgctggggggcagggggatgagcaaaggcaggcccaagacccccagcccactgcaccctggcctcccacaagccccctcgcagccgcccagccacactcactgtgcactcagccgtcgatacactggtctgttagggagaaagtccgtcagaacaggcagctgtgtgtgtgtgtgcgtgtatgagtgtgtgtgtgtgatccctgactgccaggtcctctgcactgcccctggg

SEQ ID NO: 35 - DNA, 1193 bp, Homo sapiens
  misc_feature: sequence of STAR35
cgacttggtgatgcgggctcttttttggttccatatgaactttaaagtagtcttttccaattctgtgaagaaagtcattggtaggttgatggggatggcattgaatctgtaaattaccttgggcagtatggccattttcacaatgttgattcttcctatccatgatgatggaatgttcttccattagtttgtatcctcttttatttccttgagcagtggtttgtagttctccttgaagaggtccttcacatcccttgtaagttggattcctaggtattttattctctttgaagcaaattgtgaatgggagtncactcacgatttggctctctgtttgtctgctgggtgtataaanaatgtngtgatnttngtacattgatttngtatccntgagacttngctgaatttgcttnatcngcttnngggaaccttttgggctgaaacnatgggattttctaaatatacaatcatgtcgtctgcaaacagggaacaatttgacttcctcttttcctaattgaatacactttatctccttctcctgcctaattgccctgggcaaaacttccaacactatgntngnaataggagntggtgagagagggcatccctgttcttgttgccagnttttcaaagggaatgcttccagttttggcccattcagtatgatatgggctgtgggtngtgtcataaatagctcttatnattttgaaatgtgtcccatcaatacctaatttattgaaagtttttagcatgaangcatngttgaatttggtcaaaggctttttctgcatctatggaaataatcatgtggtttttgtctttggctcntgtttatatgctggatnacatttattgatttgtgtatatngaacccagcctnncatcccagggatgaagcccacttgatccaagcttggcgcgcngnctagctcgaggcaggcaaaagtatgcaaagcatgcatctcaattagtcagcacccatagtccgcccctacctccgcccatccgcccctaactcngnccgttcgcccattctcgcccatggctgactaatntttttannatccaagcggngccgccctgcttgancattcagagtnnagagnnttggaggccnagccttgcaaaactccggacngnttctnnggattgaccccnnttaaatatttggttttttgtnttttcannggnga

SEQ ID NO: 36 - DNA, 1712 bp, Homo sapiens
  misc_feature: sequence of STAR36
gatcccatccttagcctcatcgatacctcctgctcacctgtcagtgcctctggagtgtgtgtctagcccaggcccatcccctggaactcaggggactcaggactagtgggcatgtacacttggcctcaggggactcaggattagtgagccccacatgtacacttggcctcagtggactcaggactagtgagccccacatgtacacttggcctcaggggactcaggattagtgagcccccacatgtacacttggcctcaggggactcaggattagtgagccccacatgtacacttggcctcaggggactcaggactagtgagccccacatgtacacttggcctcaggggactcagaactagtgagccccacatgtacacttggcttcaggggactcaggattagtgagccccacatgtacacttggacacgtgaaccacatcgatgtgctgcagagctcagccctctgcagatgaaatgtggtcatggcattccttcacagtggcacccctcgttccctccccacctcatctcccattcttgtctgtcttcagcacctgccatgtccagccggcagattccaccgcagcatcttctgcagcacccccgaccacacacctccccagcgcctgcttggccctccagcccagctcccgcctttcttccttggggaagctccctggacagacaccccctcctcccagccatggctttttcctgctctgccccacgcgggaccctgccctggatgtgctacaatagacacatcagatacagtccttcctcagcagccggcagacccagggtggactgctcggggcctgcctgtgaggtcacacaggtgtcgttaacttgccatctcagcaactagtgaatatgggcagatgctaccttccttccggttccctggtgagaggtactggtggatgtcctgtgttgccggccaccttttgtccctggatgccatttatttttttccacaaatatttcccaggtctcttctgtgtgcaaggtattagggctgcagcgggggccaggccacagatctctgtcctgagaagacttggattctagtgcaggagactgaagtgtatcacaccaatcagtgtaaattgttaactgccacaaggagaaaggccaggaaggagtggggcatggtggtgttctagtgttacaagaagaagccagggagggcttcctggatgaagtggcatctgacctgggatctggaggaggagaaaaatgtcccaaaagagcagagagcccaccctaggctctgcaccaggaggcaacttgctgggcttatggaattcagagggcaagtgataagcagaaagtccttgggggccacaattaggatttctgtcttctaaagggcctctgccctctgctgtgtgaccttgggcaagttacttcacctctagtgctttggttgcctcatctgtaaagtggtgaggataatgctatcacactggttgagaattgaagtaattattgctgcaaagggcttataagggtgtctaatactagtactagtaggtacttcatgtgtcttgacaattttaatcattattattttgtcatcaccgtcactcttccaggggactaatgtccctgctgttctgtccaaattaaacattgtttatccctgtgggcatctggcgaggtggctaggaaagcctggagctgtttcctgttgacgtgccagactagt

SEQ ID NO: 37 - DNA, 1321 bp, Homo sapiens
  misc_feature: sequence of STAR37
aggatcacatttaaggaagtgtgtggggtccctggatgacaccagcacccagtgcggctctgtctggcaaccgctcccaaggtggcaggagtgggtgtcccctgtgtgtcagtgggcagctcctgctgagcctacagctcactggggagcctgacagcggggccatgtgcctgacactcctctctgcttgtggacctggcaaggcagggagcagaaaacagagccacttgaaggctttctgtctgcgtctgtgtgcagtgtggatttagttgtgcttttttcttgctgggagagcacagccaccatttacaagcagtgtcaccctcatgggtggcgaggacagaacaggagcctctgctctctgtacctatctgggcccggtgggctcccttgtcctggcttccatctctgtctcagcgaccattcagccctgcgcaggaacacatgttgcttagaaaagccaaattcagcccttgtctctgcctcctctggtctcatgatgtgcatctgttaccttgaaactggaaaccagtctatcaatgtctgtgccaattttttattccctccccaacctccttccccatacgactttttatttatgtaggatgtgtgctgtctaatgatgggatgaccacatttttccatgttctaaaagtgctcctctcccgcagggtcccagggctggtggttgctttgggtctacagctacgtcttacccgcctcctgcctcaacagcctgtgtggtggcaaagccggtgtggggctggggaacgcagcgttctccaggagggggacccggctctccttctgcagtgcaggcgaaggcctagatgccagtgtgacctcccacaaggcgtggcttccagactccccggctggaagtgatgcttttttgcctccggccctgggtttgaagcagcctggctttctcttggtaagtggctggtgtcttagcagctgcaatctgagctcagccacctacacaccaccgtggccgacactttcattaaaaagtttcctgagacgacttgcgtgcatgttgacttcatgatcagcgccgctgggaagaacccctgagccggtggggtggggctggaagcagcaggtgcagtgatggggctgggtgcccaggaggcctcagtgctcaatcaggccaaggtggccaagcccaggctgcagggaaggccggcctgggggttgtgggtgagcacaggcaggcaccagctgggcagtgttaggatgctggagcagcatccgtaaccccactgagtggggtagtctggttggggcagggaccgctgttgctttggcagagagagat

SEQ ID NO: 38 - DNA, 1445 bp, Homo sapiens
  misc_feature: sequence of STAR38
gatctatgggagtagcttccttagtgagctttcccttcaaatactttgcaaccaggtagagaattttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtatnagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttgtttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaaatcctaaagacaatacatttttattaactatagccctcatgatgtacattagatcgtgtggttgtttcttccgtccccgccacgccttcctcctgggatggggattcattccctagcaggtgtcggagaactggcgcccttgcagggtaggtgccccggagcctgaggcgggnactttaanatcagacgcttgggggccggctgggaaaaactggcggaaaatattataactgnactctcaatgccagctgttgtagaagctcctgggacaagccgtggaagtcccctcaggaggcttccgcgatgtcctaggtggctgctccgcccgccacggtcatttccattgactcacacgcgccgcctggaggaggaggctgcgctggacacgccggtggcgcctttgcctgggggagcgcagcctggagctctggcggcagcgctgggagcggggcctcggaggctgggcctggggacccaaggttgggcggggcgcaggaggtgggctcagggttctccagagaatccccatgagctgacccgcagggcggccgggccagtaggcaccgggcccccgcggtgacctgcggacccgaagctggagcagccactgcaaatgctgcgctgaccccaaatgctgtgtcctttaaatgttttaattaagaataattaataggtccgggtgtggaggctcaagccttaatccccagcacctggcgaggccgaggaggga

SEQ ID NO: 39 - DNA, 2331 bp, Homo sapiens
  misc_feature: sequence of STAR39
gtgaaatagatcactaaagctgattcctcttgtctaaatgaaactttctaccctttgatggacagctatgctttccccatcctctcccgtcccccagcccttggtaaccatcatcctactctctacttgtaggagttcaacttgtttagattttgtgagtgagaacatgtggtatttgcctttagagtcctctaggtttatccatattgtgttaaatgacaggattccctgcctttttaaggctgaatagtatttcattgtaatatatatacatacacacacacatatacacacacatatatatacatatatacatatatgtacatagatacatatatatgtacatatatacacacacatatacacacatatatacacatatatacatatacatatatacacatatatgtacatatatataactttttttcatttatccattcacttaatacatatgatggagggctttatatatgccaggctctgtgatgaatgctggaaattcaatagtgagaaagactcagtctctgcctccaaagagcatcatgggctaggtgctgcaacgaggaattgccaactgttgtcatgagagcacagagaagggactcaaccagccttgaagaatcaggggaggcttctaagctaatggtgtgtgcctggggatcacattgtttcaagcagcagtaacaggatgtgctcaggtccagatgtgagagagagagagagcatatgtcttcaagaaactaacagtagctccctatagctgaagcaggagtacaaaatagtgagtttaagtgatgaggcaagagatatgaagaagcttgaccatgcagctacaccgggcagcatgccctctgagacatctcatggaagccggaaatgggagtgccttgataccaagccagagaaattataatactaagtagatagactgagcagcactcctcctgggaagaatgagacaagccctgaatttggaggtaagttgtggattggtgattagaggagaggtaacaggcaccaaagcaagaaatagtattgatgcaaagctgaggttaattggatgacaaaatgaagagcataaggggctcagacacagactgagcagaaaacgagtagcatctgaacctagattgagttactaatggatgagaaagagttcttaaagttgatgaccacgggatccatatataagaatgtccaatctccccaaattgatccacgagttcagtgcaatgccaatcaaaatcccactaacaagtttattttaaaatgtaaatgaaaatacaaaatttttaaaaagcaaagcaatattgaaaacccaggaaaaattaggaggacttacacaacctgatctcaaaacttaccattatcaagacagagtgttattgacacaaggagagacaaatagataaacggaatgtggtagtctggagatgcacccacatgtatgtggtcaattgatttttggccaaggcaccaagtcaattcaaaggagcaaggaaagtagtacagaaacaaccaaatattgttttggaaaataatgacaaagggcttataaccagaatataagcatataaatataattctttcaaatcaataataagaaggcaaatatctaataaaaatgagcaaagacttgaaaagtcacttaaaaaggcttattaattagaaatatgcaaatgttattagtcttcagtggaatttacattaaaccacaagggatactattatatcttatgcccactagaataaccaaaggaaaaaagacagacaaaacaaaatgctggtgaggatgtgaagcaactggaactctcatacattattggtggtaatgtaaaatttatacaaccattatgaataaaggtttggcagtttcttacaaagttgaatgcacttctccacgatgactaggcttttcactcataggcgtctggctccctagaactgaaaacatatgttcacaagaagacttgcaaatatatattctcccacgtcaggagatatttgctatgcatttaactgacataagattagtgctagagtttataatgaggttcttcaaatctaaaagaaaatgcaaagcatataatagtaaggggtgcaggccaggcgcagtggctcactctgtaatcccagcactttgggaggccgaggtgggcggatcacaaggtcaggagttcgagaccaacctggccaacatagtgaaaccctgtctctactaaaaatacaaaaactagccaggtgcggtgtcatgcacctgtagtcccagctactcgggaggccgaggcaggagaatcacttgaacctgggaggtggaggttgcagtga

SEQ ID NO: 40 - DNA, 1071 bp, Homo sapiens
  misc_feature: sequence of STAR40
gctgtgattcaaactgtcagcgagataaggcagcagatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaattttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttgtttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaaatcctaaagacaatacatttttattaactatagccctcatgatgtacattagatctctaa

SEQ ID NO: 41 - DNA, 1135 bp, Homo sapiens
  misc_feature: sequence of STAR41
cgtgtgcagtccacggagagtgtgttctcctcatcctcgttccggtggttgtggcgggaaacgtggcgctgcaggacaccaacatcagtcacgtatttcattctggaaaaaaaagtagcacaagcctcggctggttccctccagctcttaccaggcagcctaagcctaggctccattcccgctcaaggccttcctcaggggcctgctcaccacaggagctgttcccatgcagggactaaggacatgcagcctgcatagaaaccaagcacccaggaaaacatgattggatggagcgggggggtgtggtctctagccttgtccacctccggtcctcatgggtctcacacctcctgagaatgggcaccgcagaggccacagcccatacagccaagatgacagactccgtaagtgacagggatccacagcagagtgggtgaaatgttccctataaactttacaaaattaatgagggcagggggaggggagaaatgaaaatgaacccagctcgcagcacatcagcatcagtcactaggtcggcgtgctctctgactgcttcctcgtagctgcttggtgtctcattgcctcagaagcatgtagaccctgtcacaagattgtagttcccctaactgctccgtagatcacaacttgaaccttaggaaatgctgttttccctttgagatattcctttgggtcctgtatactgatggagctactgactgagctgctccgaaggaccccacgaggagctgactaaaccaagagtgcagtttgtacaccctgatgattacatcccccttgccccaccaatcaactctcccaattttccagcccctcaccctccagtccccttaaaagccccagcccaggccgggcacagtggctcatgcctgtaatcccagcactttgggaggccaaggtgggcagatcacctgagggcaggaatttgagaccagcctgaccaacatgaagaaaccccgtctctattacaaatacaaaattagccgggcgtgttgctgcatactggtaatcccagctacttgggagggtgaggcaggagaatcacttgaatctgggaggcggaggttgcgatgagccgagacagcgccattgcactgcagcctgggcaacaagagca

SEQ ID NO: 42 - DNA, 735 bp, Homo sapiens
  misc_feature: sequence of STAR42
aagggtgagatcactagggagggaggaaggagctataaaagaaagaggtcactcatcacatcttacacactttttaaaaccttggttttttaatgtccgtgttcctcattagcagtaagccctgtggaagcaggagtctttctcattgaccaccatgacaagaccctatttatgaaacataatagacacacaaatgtttatcggatatttattgaaatataggaatttttcccctcacacctcatgaccacattctggtacattgtatgaatgaatataccataattttacctatggctgtatatttaggtcttttcgtgcaggctataaaaatatgtatgggccggtcacagtgacttacgcccgtagtcccagaactttgggaggccgaggcgggtggatcacctgaggtcgggagttcaaaaccagcctgaccaacatggagaaaccccgtctctgctaaaaatacaaaaattaactggacacggtggcgtatgcctgtaatcccagctactcgggaagctgaggcaggagaactgcttgaacccaggaggcggaggttgtggtgagtcgagattgcgccattgcactccagcctgggcaacaagagcgaaattccatctcaaaaaaaagaaaaaagtatgactgtatttagagtagtatgtggatttgaaaaattaataagtgttgccaacttaccttagggtttataccatttatgagggtgtcggtttc

SEQ ID NO: 43 - DNA, 1227 bp, Homo sapiens
  misc_feature: sequence of STAR43
caaatagatctacacaaaacaagataatgtctgcccatttttccaaagataatgtggtgaagtgggtagagagaaatgcatccattctccccacccaacctctgctaaattgtccatgtcacagtactgagaccagggggcttattcccagcgggcagaatgtgcaccaagcacctcttgtctcaatttgcagtctaggccctgctatttgatggtgtgaaggcttgcacctggcatggaaggtccgttttgtacttcttgctttagcagttcaaagagcagggagagctgcgagggcctctgcagcttcagatggatgtggtcagcttgttggaggcgccttctgtggtccattatctccagcccccctgcggtgttgctgtttgcttggcttgtctggctctccatgccttgttggctccaaaatgtcatcatgctgcaccccaggaagaatgtgcaggcccatctcttttatgtgctttgggctattttgattccccgttgggtatattccctaggtaagacccagaagacacaggaggtagttgctttgggagagtttggacctatgggtatgaggtaatagacacagtatcttctctttcatttggtgagactgttagctctggccgcggactgaattccacacagctcacttgggaaaactttattccaaaacatagtcacattgaacattgtggagaatgagggacagagaagaggccctagatttgtacatctgggtgttatgtctataaatagaatgctttggtggtcaactagacttgttcatgttgacatttagtcttgccttttcggtggtgatttaaaaattatgtatatcttgtttggaatatagtggagctatggtgtggcattttcatctggctttttgtttagctcagcccgtcctgttatgggcagccttgaagctcagtagctaatgaagaggtatcctcactccctccagagagcggtcccctcacggctcattgagagtttgtcagcaccttgaaatgagtttaaacttgtttatttttaaaacattcttggttatgaatgtgcctatattgaattactgaacaaccttatggttgtgaagaattgatttggtgctaaggtgtataaatttcaggaccagtgtctctgaagagttcatttagcatgaagtcagcctgtggcaggttgggtggagccagggaacaatggagaagctttcatgggtgg

SEQ ID NO: 44 - DNA, 1586 bp, Homo sapiens
  misc_feature: sequence of STAR44
cacctgcctcagcctcccaaagtgctgagattcaaagaaattttcatggagaggggacagatggagtcaattcttgtggggtgaacatgagtaccacagttagactgaggttgggaaagattttccagacaattggaagagcatgtgaaagacacagattttgagaaatgttaagtctagggaactgcaaggcttttggcacaagaaagccactgtagactatagaggcaggatgcctagattcaaatcccaactgctacacttctaagctttgtaattttggcaagtttttaccctctattttcttatctataaaatatagattttatatatatagatatagatatatagatagataataattgtgcatgcctaataaagttgtcaaagattaaatgttatatgtgaagtattttgtacggtgataggaacccaggaagggctctatgaatattatgtattattattattctaaagtagctggaatacaatgttcaaaggagatagtggcaggagataagtttgaattgaaagattgaggccagaacataaagtgcctcctatattatattttacataattggaacatcattgaaaaatttaagtattatttatgtgtgtatgtgtgttttatataattaattctagttcatcattttaaaatatctttctgatgtcactgtgaacaacagatgagaagaagtgaatcctgagttaaggagaccagctctctgattactgccataatccagggagggtaccataaggatttcaactggaagtgaatccatcatgatggagaggaaggacagggctgaaaaatacttaggaagtagtatcagtaggactggttaagagagagcagaggcaggctacaggggttggaggtgtcaatcacagagatagggaaaatgggaggagaagcaggctttgaaaaagtggcttgtcttgtaaaattatgtgctgttaaaacagtacaagaaattaatatattcaatcccaaaatacagggacaattctttttgaaagagttacccagatagtcttccttgaagttttcagttaaagaaatttcttgttaacaaataatgtagtcatagaagaaaacacttaaaactttattgaataaagctaataaatcatttaatataatttataggaaattgttacataacacacacattcaatactttttgctaaagtataaattaatggaaggagagcacgcacacagaggttgaattatgtttatgactttattagtcaagaatacaaaattgagtagctacatcaagcagaagcacatgctttacaatccagcacagaatcccttgacatccaaactcccgaaacagacatgtaaatacagatgacattgtcagaacaaaatagggtctcacccgacctataatgttcttttcttgatataaatatgcacatgaattgcatacggtcatatggttccaattaccattatttcctctgggcttagctatccatctaaggggaatttacaccaacactgtacttctacttgcaagaatatatgaaagcatagttaacttctggcttaggaccccaactca

SEQ ID NO: 45 - DNA, 1981 bp, Homo sapiens
  misc_feature: sequence of STAR45
atggatcatagggtaaataaatttataatttcttgagaaagcttcgtactgttttccaagatggctgtactaatttccattcctaccaacagtgtacagggtttctttttctccacatcctcaccaacacttatcttccatctttttttataatagccctagtaaaatgtgtgaggtgatatctcattgtggcattgatttgcacttctctgataattaggaatgtttatgattttttcatgtacctggttggccttttgtatgatgtaggaaatgtctattctgattctttgcttattttttaataagcatagtttttttcttatttttgagtaggttgagttgcttatatattattatatgagccccttacctgatgtatggtttaaaaatattatcccatttgtgggttctcttaattctatcattgcttcttttcctgtggaaaagttttaagttttatgcagtctcatttgtgtgttttgcttttgttgccttttggaataatctacagaaaatcatagctcaggccaatgtcatacagtctccttctatatttccttgtagtagttttacatttaaactttaattttgatttgatgcttgtataaagagcaaaataaaagtcaaattttattcttctgtatgtggatagtcagttttgtctacaccatttattgaaaataattttctttcttcactgtgtatttttagttattttatcaaaaaatcaattgaccacagacacacggatttatttacaggttctatatccctttgtactgttttacatgtctgtttttatgccattgctatgctgttttaattcctatagctttgtaatagagtttggagtcaggtagtctgatgcctccagctttgttctttttgttcaagattgctttggttggtccaggtcttttgtggttccatacaaattttagcagtaatttttctatttctgtgaagaatgacattggaatttgatagtggttgcatttaatctgtagattgctttgggtagcattgacacttttacaatactaatttttgaatccatcaatgaaggatgtttctccatttatttatgccattttaatttttttcatcaatgtgctatagttttcagtatgtaaatcttttatggttttgattaaatttactcctgtcttttatatatttatatatctgttttgattctattataaattgaattgcctttatttttcaggtaatagtttgtcattagttaatagaaacaataatgatatttgtatgttgattttgtaactattaactttattgaatttcttcatcagctataaccatttattttggtggaatctttaagattttctctatcttaagattatattttcaaaaaacagaaacaatcttacctcttccttccctatgtggatttcttttacgtctttgtcttgtgtaactgttctggctaggcaattacacataatgttttcatcatttataattttacatcacatccatctattgtggcacattgattgctacttttcaagttgtaaacctggacatttatcactactcttcctccaatacaggagtccatggcgtggtgtgggccctactgtgccacagtccagggcacggctgggctgaggttctcttgtgcaagagtccgtggctctgcggagcaagagttctccagtgccttagtccagggttaggcaggggtggggctccttcagtagcttagtccagtgcgccgccctgcgagggtcctcctgagcaggagtacacgatgaggcagggtcctactgtgccttagcccaggaagcggggggctgggtcctctggtgccatagtccaggctgccgggagctgggtcctctggtgccatagctcaggccggcgggagctgggtcctctggtgccgtagtccagggtgcagcagaacaggagtcctgcggagcagtagtccagggcacgctggggcgtg

SEQ ID NO: 46 - DNA, 1859 bp, Homo sapiens
  misc_feature: sequence of STAR46
attgtttttctcgcccttctgcattttctgcaaattctgttgaatcattgcagttacttaggtttgcttcgtctcccccattacaaactacttactgggtttttcaaccctagttccctcatttttatgatttatgctcatttctttgtacacttcgtcttgctccatctcccaactcatggcccctggctttggattattgttttggtcttttattttttgtcttcttctacctcaacacttatcttcctctcccagtctccggtaccctatcaccaaggttgtcattaacctttcatattattcctcattatccatgtattcatttgcaaataagcgtatattaacaaaatcacaggtttatggagatataattcacataccttaaaattcaggcttttaaagtgtacctttcatgtggtttttggtatattcacaaagttatgcattgatcaccaccatctgattccataacatgttcaatacctcaaaaagaagtctgtactcattagtagtcatttcacattcaccactccctctggctctgggcagtcactgatctttgtgtctctatggatttgcctagtctaggtatttttatgtaaatggcatcatacaacatgtgaccttttgtttggcttttttcatttagcaaaatgttatcaaggtctgtccctgttgtagcatgtattagcacttcatttcttatatgctgaatgatatactttatttgtccatcagttgttcatgctttatttgtccatcagttgatgaacatttgcgtttttgccactttgggctattaagaataatgctactgtgaacaagtgtgtacaagttcctctacaaatttttgtgtggacatatcctttcagttctctcaggtgtatatctgggaattgaattgctgggtcgtgtagtagctatgttaaacactttgagaaactgctataatgttctccagagctgtaccattttaaattctgtgtatgaggattccacgttctccacttcctcaccagtgtatggatttgggggtatactttttaaaaagtgggattaggctgggcacagtggctcacacctgtaatcccaacacttcaggaagctgaggtgggaggatcacttgagcctagtagtttgagaccagcctgggcaacatagggagaccctgtctctacaaaaaataatttaaaataaattagctgggcgttgtggcacacacctgtagtcccagctacatgggaggctgaggtggaaggattccctgagcccagaagtttgaggttgcagtgagccatgatggcagcactatactgtagcctgggtgtcagagcaagactccgtttcagggaagaaaaaaaaaagtgggatgatatttttgacacttttcttcttgttttcttaatttcatacttctggaaattccattaaattagctggtaccactctaactcattgtgtttcatggctgcatagtaatattgcataatataaatataccattcattcatcaaagttagcagatattgactgttaggtgccaggcactgctctaagcgttaaagaaaaacacacaaaaacttttgcattcttagagtttattttccaatggagggggtggagggaggtaagaatttaggaaataaattaattacatatatagcatagggtttcaccagtgagtgcagcttgaatcgttggcagctttcttagtagtataaatacagtactaaagatgaaattactctaaatggtgttacttaaattactggaataggtattactattagtcactttgcaggtgaaagtggaaacaccatcgtaaaatgtaaaataggaaacagctggttaatgtt

SEQ ID NO: 47 - DNA, 1082 bp, Homo sapiens
  misc_feature: sequence of STAR47
atcattagtcattagggaaatgcaaatgaaaaacacaagcagccaccaatatacacctactaggatgatttaaaggaaaataagtgtgaagaaggacgtaaagaaattgtaaccctgatacattgatggtagaaatggataaagttgcagccactgtgaaaaacagtctgcagtggctcagaaggttaaatatagaacccctgttggacccaggaactctactcttaggcaccccaaagaatagagaacagaaatcaaacagatgtttgtatactaatgtttgtagcatcacttttcacaggagccaaaaggtggaaataatccaaccatcagtgaacaaatgaatgtaataaaagcaaggtggtctgcatgcaatgctacatcatccatctgtaaaaaacgaacatcattttgatagatgatacaacatgggtggacattgagaacattatgcttagtgaaataagccagacacaaaaggaatatattgtataattgtaattacatgaagtgcctagaatagtcaaattcatacaagagaaagtgggataggaatcaccatgggctggaaatagggggaaggtgctatactgcttattgtggacaaggtttcgtaagaaatcatcaaaattgtgggtgtagatagtggtgttggttatgcaaccctgtgaatatattgaatgccatggagtgcacactttggttaaaaggttcaaatgataaatattgtgttatatatatttccccacgatagaaaacacgcacagccaagcccacatgccagtcttgttagctgccttcctttaccttcaagagtgggctgaagcttgtccaatctttcaaggttgctgaagactgtatgatggaagtcatctgcattgggaaagaaattaatggagagaggagaaaacttgagaatccacactactcaccctgcagggccaagaactctgtctcccatgctttgctgtcctgtctcagtatttcctgtgaccacctcctttttcaactgaagactttgtacctgaaggggttcccaggtttttcacctcggcccttgtcaggactgatcctctcaacta

SEQ ID NO: 48 - DNA, 1242 bp, Homo sapiens
  misc_feature: sequence of STAR48
atcatgtatttgttttctgaattaattcttagatacattaatgttttatgttaccatgaatgtgatattataatataatatttttaattggttgctactgtttataagaatttcattttctgtttactttgccttcatatctgaaaaccttgctgatttgattagtgcatccacaaattttcttggattttctatgggtaattacaaatctccacacaatgaggttgcagtgagccaagatcacaccactgtactccagcctgggcgacagagtgagacaccatctcacaaaaacacataaacaaacaaacagaaactccacacaatgacaacgtatgtgctttctttttttcttcctctttctataatatttctttgtcctatcttaactgaactggccagaaaccccaggacaatgataaatacgagcagtgtcaacagacatctcattccctttcctagcttttataaaaataacgattatgcttcaacattacatatggtggtgtcgatggttttgttatagataagcttatcaggttaagaaatttgtctgcgtttcctagtttggtataaagattttaatataaatgaatgttgtattttatcatcttatttttttcctacatctgctaaggtaatcctgtgttttccccttttcaatctcctaatgtggtgaatgacattaaaataccttctattgttaaaatattcttgcaacgctgtatagaaccaatgcctttattctgtattgctgatggatttttgaaaaatatgtaggtggacttagttttctaaggggaatagaatttctaatatatttaaaatattttgcatgtatgttctgaaggacattggtgtgtcatttctataccatctggctactagaggagccgactgaaagtcacactgccggaggaggggagaggtgctcttccgtttctggtgtctgtagccatctccagtggtagctgcagtgataataatgctgcagtgccgacagttctggaaggagcaacaacagtgatttcagcagcagcagtattgcgggatccccacgatggagcaagggaaataattctggaagcaatgacaatatcagctgtggctatagcagctgagatgtgagttctcacggtggcagcttcaaggacagtagtgatggtccaatggcgcccagacctagaaatgcacatttcctcagcaccggctccagatgctgagcttggacagctgacgcct

SEQ ID NO: 49 - DNA, 1015 bp, Homo sapiens
  misc_feature: sequence of STAR49
aaaccagaaacccaaaacaatgggagtgacatgctaaaaccagaaacccaaaacaatgggagggtcctgctaaaccagaaacccaaaacaatgggagtgaagtgctaaaaccagaaacccaaaacaatgggagtgtcctgctacaccagaaacccaaaacgatgggagtgacgtgataaaaccagacacccaaaacaatgggagtgacgtgctaaaccagaaacccaaaacaatgggagtgacgtgctaaaacctggaaacctaaaacaatgcgagtgaggtgctaacaccagaatccataacaatgtgagtgacgtgctaaaccagaacccaaaacaatgggagtgacgtgctaaaacaggaacccaaaacaatgagagtgacgtgctaaaccagaaacccaaaacaatgggaatgacgtgctaaaaccggaacccaaaacaatgggagtgatgtgctaaaccagaaacccaaaacaatgggaatgacatgctaaaactggaacccaaaacaatggtaactaagagtgatgctaaggccctacattttggtcacactctcaactaagtgagaacttgactgaaaaggaggatttttttttctaagacagagttttggtctgtcccccagagtggagtgcagtggcatgatctcggctcactgcaagctctgcctcccgggttcaggccattctcctgcctcagcctcctgagtagctgggaatacaggcacccgccaccacacttggctaattttttgtatttttagtagagatggggtttcaccatattagcaaggatggtctcaatctcctgacctcgtgatctgcccacctcaggctcccaaagtgctgggattacaggtgtgagccaccacacccagcaaaaaggaggaatttttaaagcaaaattatgggaggccattgttttgaactaagctcatgcaataggtcccaacagaccaaaccaaaccaaaccaaaatggagtcactcatgctaaatgtagcataatcaaa

SEQ ID NO: 50 - DNA, 2355 bp, Homo sapiens
  misc_feature: sequence of STAR50
caaccatcgttccgcaagagcggcttgtttattaaacatgaaatgagggaaaagcctagtagctccattggattgggaagaatggcaaagagagacaggcgtcattttctagaaagcaatcttcacacctgttggtcctcacccattgaatgtcctcacccaatctccaacacagaaatgagtgactgtgtgtgcacatgcgtgtgcatgtgtgaaagtatgagtgtgaatgtgtctatatgggaacatatatgtgattgtatgtgtgtaactatgtgtgactggcagcgtggggagtgctggttggagtgtggtgtgatgtgagtatgcatgagtggctgtgtgtatgactgtggcgggaggcggaaggggagaagcagcaggctcaggtgtcgccagagaggctgggaggaaactataaacctgggcaatttcctcctcatcagcgagcctttcttgggcaataggggcagagctcaaagttcacagagatagtgcctgggaggcatgaggcaaggcggaagtactgcgaggaggggcagagggtctgacacttgaggggttctaatgggaaaggaaagacccacactgaattccacttagccccagaccctgggcccagcggtgccggcttccaaccataccaaccatttccaagtgttgccggcagaagttaacctctcttagcctcagtttccccacctgtaaaatggcagaagtaaccaagcttaccttcccggcagtgtgtgaggatgaaaagagctatgtacgtgatgcacttagaagaaggtctagggtgtgagtggtactcgtctggtgggtgtggagaagacattctaggcaatgaggactggggagagcctggcccatggcttccactcagcaaggtcagtctcttgtcctctgcactcccagccttccagagaggaccttcccaaccagcactccccacgctgccagtcacacatagttacacacatacaatcacatatatgttcccatatagacacattcacactcataccttcacacatgcacacgcatgtgcacacacagtcactcatttctgtgttggagattgggtgaggacattcaatgggtgaggaccaacaggtgtgaagattgctttctagaaaatgactcctgtctctctttgccattcttcccaatccgatggagctactaggcttttccctcatttcatgtttaataaaccttcccaatggcgaaatgggctttctcaagaagtggtgagtgtcccatccctgcggtggggacaggggtggcagcggacaagcctgcctggagggaactgtcaggctgattcccagtccaactccagcttccaacacctcatcctccaggcagtcttcattcttggctctaatttcgctcttgttttcttttttatttttatcgagaactgggtggagagcttttggtgtcattggggattgctttgaaacccttctctgcctcacactgggagctggcttgagtcaactggtctccatggaatttctttttttagtgtgtaaacagctaagttttaggcagctgttgtgccgtccagggtggaaagcagcctgttgatgtggaactgcttggctcagatttcttgggcaaacagatgccgtgtctctcaactcaccaattaagaagcccagaaaatgtggcttggagaccacatgtctggttatgtctagtaattcagatggcttcacctgggaagccctttctgaatgtcaaagccatgagataaaggacatatatatagtagctagggtggtccacttcttaggggccatctccggaggtggtgagcactaagtgccaggaagagaggaaactctgttttggagccaaagcataaaaaaaccttagccacaaaccactgaacatttgttttgtgcaggttctgagtccagggagggcttctgaggagaggggcagctggagctggtaggagttatgtgagatggagcaagggccctttaagaggtgggagcagcatgagcaaaggcagagaggtggtaatgtataaggtatgtcatgggaaagagtttggctggaacagagtttacagaatagaaaaattcaacactattaattgagcctctactacgtgctcgacattgttctagtcactgagataggtttggtatacaaaacaaaatccatcctctatggacattttagtgactaacaacaatataaataataaaagtgaacaaaagctcaaaacatgccaggcactattatttatttatttatttatttatttatttattttttgaaacagagtctcgctctgttgcccaggctggagtgtagtggtgcgatctcggctcactg

SEQ ID NO: 51 - DNA, 2289 bp, Homo sapiens
  misc_feature: sequence of STAR51
tcacaggtgacaccaatcccctgaccacgctttgagaagcactgtactagattgactttctaatgtcagtcttcattttctagctctgttacagccatggtctccatattatctagtacaacacacatacaaatatgtgtgatacagtatgaatataatataaaaatatgtgttataatataaatataatattaaaatatgtctttatactagataataatacttaataacgttgagtgtttaactgctctaagcactttacctgcaggaaacagttttttttttattttggtgaaatacaactaacataaatttatttacaattttaagcatttttaagtgtatagtttagtggagttaatatattcaaaatgttgtgcagccgtcaccatcatcagtcttcataactcttttcatattgtaaaattaaaagtttatgctcatttaaaaatgactcccaatttcccccctcctcaacctctggaaactaccattctattttctgcctccgtagttttgcccactctaagtacctcacataagtggaatttgtcttatttgcctgtttgtgaccggctgatttcatttagtataatgtcctcaagttttattcacgttatatagcatatgtcataattttcttcacttttaagcttgagtaatatttcatcgtatgtatctcacattttgcttatccattcatctctcagtggacacttgagttgcttctacattttagctgttgtgaatactgctgctatgaacatgggtgtataaatatctcaagacctttttatcagttttttaaaatatatactcagtagtagtttagctggattatatggtaattttatttttaatttttgaggaactgtcctacccttttattcaatagtagctataccaattgacaattggcattcctaccaacagggcataagggttctcaattctccacatattccctgatacttgttattttcaggtgttttttttttttttttttttttatgggagccatgttaatgggtgtaaggtgatatttcattatagttttgatttgcatttccctaatgattagtgatgttaagcatctcttcatgtgcctattggccatttgtatatcttctttaaaaatatatatatactcattcctttgcccatttttgaattatgtttattttttgttattgagtttcaatacttttctatataacctaggtattaatcctttatcagacttaagatttgcaaatattctctttcattccacaggttgctaattctctctgttggtaatatcttttgatgctgttgtgtccagaattgattcattcctgtgggttcttggtctcactgacttcaagaataaagctgcggaccctagtggtgagtgttacacttcttatagatggtgtttccggagtttgttccttcagatgtgtccagagtttcttccttccaatgggttcatggtcttgctgacttcaggaatgaagccgcagaccttcgcagtgaggtttacagctcttaaaggtggcgtgtccagagttgtttgttccccctggtgggttcgtggtcttgctgacttcaggaatgaagccgcagaccctcgcagtgagtgttacagctcataaaggtagtgcggacacagagtgagctgcagcaagatttactgtgaagagcaaaagaacaaagcttccacagcatagaaggacaccccagcgggttcctgctgctggctcaggtggccagttattattcccttatttgccctgcccacatcctgctgattggtccattttacagagtactgattggtccattttacagagtgctgattggtgcatttacaatcctttagctagacacagagtgctgattgctgcattcttacagagtgctgattggtgcatttacagtcctttagctagatacagaacgctgattgctgcgttttttacagagtgctgattggtgcatttacaatcctttagctagacacagtgctgattggtgggtttttacagagtgctgattggtgcgtctttacagagtgctgattggtgcatttacaatcctttagctagacacagagtgctgattggtgcgtttataatcctctagctagacagaaaagttttccaagtccccacctgaccgagaagccccactggcttcacctctcactgttatactttggacatttgtccccccaaaatctcatgttgaaatgtaacccctaatgttggaactgaggccagactggatgtggctgggccatgggga

SEQ ID NO: 52 - DNA, 1184 bp, Homo sapiens
  misc_feature: sequence of STAR52
ctcttctttgtttttttattttggggtgtgtgggtacgtgtaagatgagaaatgtacaaacacaagtatttcagaaactccaagtaatattctgtctgtgagttcacggtaaataaataaaaagggcaaagtgacagaaatacaggattattaaaagcaaaataatgttctttgaaatcccccccttggtgtattttttatcttaggatgcagcactttcagcatgcccaagtattgaaagcagtgtttttacgctaccacggtaattttatttagaaaccccatgttcacttttagttttaaaatggtctttatgacataaaattatcagcattcatatttttgtgttttaatattcctttggctacttattgaaacagtaaacattacgaaaattagtaaacaaatctttgatagttgcttatttttgtttaattgaatgtttattttattaggtaaatatacaatcaaatttatttaaaaataatgaggaaaagaatacttttctttcgctttgcgaaagcaaagtgatttttcattcttctccgtccgattccttctcttccagctgccacagccgactgacaggctcccggcggcctgaggagtagtatgcaaattttggatgattgacacctacagtagaagccaatcacgtcaaagtaggatgctgattggttgacaacaataggcgtaaaccttgacgttttaaaaacctgacacccaatccaggcgattcatgcaaataaaggaagggagtcacattaccaggggccagagagacttgagtacgacctcacgtgttcagtggtggatattgcacagacgtctgcaaggtctatataaacgctacataatgttcaactcaattgcttgccttggcctttcccaaacttgtcactggaatataaattatcccttttttaaaaataaaaaaataagaattatgtagtgcacatatatgatggttcatgtagaaatctaaatggacttccaacgcatggaattttcctatttccccctttctttaaattaatcctcagtgaaggaggctgttttcccctagatttcaaaaggacgagatttacagagcctttccttggagaaacccgctctaggcacagatggtcagtaaatttagcttcttcagcgaagttccacatggcaccgccagatggcataag

SEQ ID NO: 53 - DNA, 1431 bp, Homo sapiens
  misc_feature: sequence of STAR53
ccctgaggaagatgacgagtaactccgtaagagaaccttccactcatcccccacatccctgcagacgtgctattctgttatgatactggtatcccatctgtcacttgctccccaaatcattcccttcttacaattttctactgtacagcattgaggctgaacgatgagagatttcccatgctctttctactccctgccctgtatatatccggggatcctccctacccaggatgctgtggggtcccaaaccccaagtaagccctgatatgcgggccacacctttctctagcctaggaattgataacccaggcgaggaagtcactgtggcatgaacagatggttcacttcgaggaaccgtggaaggcgtgtgcaggtcctgagatagggcagaatcggagtgtgcagggtctgcaggtcaggaggagttgagattgcgttgccacgtggtgggaactcactgccacttatttccttctctcttcttgcctcagcctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttccccagttgtttgctcgtgccattattgtgcacacagctctgtgcacgtgtgtgcatatttctttaggaaagattcttagaagtggaattgctgtgtcaaaggagtcatttattcaacaaaacactaatgagtgcgtcctcgtgctgagcgctgttctaggtgctggagcgacgtcagggaacaaggcagacaggagttcctgacccccgttctagaggaggatgtttccagttgttgggttttgtttgtttgtttcttctagagatggtggtcttgctctgtccaggctagagtgcagtggcatgatcatagc

SEQ ID NO: 54 - DNA, 975 bp, Homo sapiens
  misc_feature: sequence of STAR54
ccataaaagtgtttctaaactgcagaaaaatccccctacagtcttacagttcaagaattttcagcatgaaatgcctggtagattacctgactttttttgccaaaaataaggcacagcagctctctcctgactctgactttctatagtccttactgaattatagtccttactgaattcattcttcagtgttgcagtctgaaggacacccacattttctctttgtctttgtcaattctttgtgttgtaagggcaggatgtttaaaagttgaagtcattgacttgcaaaatgagaaatttcagagggcattttgttctctagaccatgtagcttagagcagtgttcacactgaggttgctgctaatgtttctgcagttcttaccaatagtatcatttacccagcaacaggatatgatagaggacttcgaaaaccccagaaaatgttttgccatatatccaaagccctttgggaaatggaaaggaattgcgggctcccatttttatatatggatagatagagaccaagaaagaccaaggcaactccatgtgctttacattaataaagtacaaaatgttaacatgtaggaagtctaggcgaagtttatgtgagaattctttacactaattttgcaacattttaatgcaagtctgaaattatgtcaaaataagtaaaaatttttacaagttaagcagagaataacaatgattagtcagagaaataagtagcaaaatcttcttctcagtattgacttggttgcttttcaatctctgaggacacagcagtcttcgcttccaaatccacaagtcacatcagtgaggagactcagctgagactttggctaatgttggggggtccctcctgtgtctccccaggcgcagtgagcctgcaggccgacctcactcgtggcacacaactaaatctggggagaagcaacccgatgccagcatgatgcagatatctcagggtatgatcggcc

SEQ ID NO: 55 - DNA, 501 bp, Homo sapiens
  misc_feature: sequence of STAR55
cctgaactcatgatccgcccacctcagcctcctgaagtgctgggattacaggtgtgagccaccacacccagccgcaacacactcttgagcaaccaatgtgtcataaaagaaataaaatggaaatcagaaagtatcttgagacagacaaaaatggaaacacaacataccaaaatttatgggacacagcaaaagcagttttaggagggaagtttatagtgatgaatacctacctcaaaatcattagcctgattggatgacactacagtgtataaatgaattgaaaaccacattgtgccccatacatatatacaatttttatttgttaattaaaaataaaataaaactttaaaaaagaagaaagagctcaaataaacaacctaactttatacctcaaggaaatagaagagccagctaagcccaaagttgacagaaggaaaaaaatattggcagaaagaaatgaaacagagactagaaagacaattgaagagatcagcaaaacta

SEQ ID NO: 56 - DNA, 741 bp, Homo sapiens
  misc_feature: sequence of STAR56
acacaggaaaagatcgcaattgttcagcagagctttgaaccggggatgacggtctccctcgttgcccggcaacatggtgtagcagccagccagttatttctctggcgtaagcaataccaggaaggaagtcttactgctgtcgccgccggagaacaggttgttcctgcctctgaacttgctgccgccatgaagcagattaaagaactccagcgcctgctcggcaagaaaacgatggaaaatgaactcctcaaagaagccgttgaatatggacgggcaaaaaagtggatagcgcacgcgcccttattgcccggggatggggagtaagcttagtcagccgttgtctccgggtgtcgcgtgcgcagttgcacgtcattctcagacgaaccgatgactggatggatggccgccgcagtcgtcacactgatgatacggatgtgcttctccgtatacaccatgttatcggagagctgccaacgtatggttatcgtcgggtatgggcgctgcttcgcagacaggcagaacttgatggtatgcctgcgatcaatgccaaacgtgtttaccggatcatgcgccagaatgcgctgttgcttgagcgaaaacctgctgtaccgccatcgaaacgggcacatacaggcagagtggccgtgaaagaaagcaatcagcgatggtgctctgacgggttcgagttctgctgtgataacggagagagactgcgtgtcacgttcgcgctggactgctgtg

SEQ ID NO: 57 - DNA, 1365 bp, Homo sapiens
  misc_feature: sequence of STAR57
tccttctgtaaataggcaaaatgtattttagtttccaccacacatgttcttttctgtagggcttgtatgttggaaattttatccaattattcaattaacactataccaacaatctgctaattctggagatgtggcagtgaataaaaaagttatagtttctgattttgtggagcttggactttaatgatggacaaaacaacacattcttaaatatatatttcatcaaaattatagtgggtgaattatttatatgtgcatttacatgtgtatgtatacataaatgggcggttactggctgcactgagaatgtacacgtggcgcgaacgaggctgggcggtcagagaaggcctcccaaggaggtggctttgaagctgagtggtgcttccacgtgaaaaggctggaaagggcattccaagaaaaggctgaggccagcgggaaagaggttccagtgcgctctgggaacggaaagcgcacctgcctgaaacgaaaatgagtgtgctgaaataggacgctagaaagggaggcagaggctggcaaaagcgaccgaggaggagctcaaaggagcgagcggggaaggccgctgtggagcctggaggaagcacttcggaagcgcttctgagcgggtaaggccgctgggagcatgaactgctgagcaggtgtgtccagaattcgtgggttcttggtctcactgacttcaagaatgaagagggaccgcggaccctcgcggtgagtgttacagctcttaaggtggcgcgtctggagtttgttccttctgatgttcggatgtgttcagagtttcttccttctggtgggttcgtggtctcgctggctcaggagtgaagctgcagaccttcgcggtgagtgttacagctcataaaagcagggtggactcaaagagtgagcagcagcaagatttattgcaaagaatgaaagaacaaagcttccacactgtggaaggggaccccagcgggttgccactgctggctccgcagcctgcttttattctcttatctggccccacccacatcctgctgattggtagagccgaatggtctgttttgacggcgctgattggtgcgtttacaatccctgcgctagatacaaaggttctccacgtccccaccagattagctagatagagtctccacacaaaggttctccaaggccccaccagagtagctagatacagagtgttgattggtgcattcacaaaccctgagctagacacagggtgatgactggtgtgtttacaaaccttgcggtagatacagagtatcaattggcgtatttacaatcactgagctaggcataaaggttctccaggtccccaccagactcaggagcccagctggcttcacccagtgg

SEQ ID NO: 58 - DNA, 1401 bp, Homo sapiens
  misc_feature: sequence of STAR58
aagtttaccttagccctaaattatttcattgtgattggcattttaggaaatatgtattaaggaatgtctcttaggagataaggataacatatgtctaagaaaattatattgaaatattattacatgaactaaaatgttagaactgaaaaaaaattattgtaactccttccagcgtaggcaggagtatctagataccaactttaacaactcaactttaacaacttcgaaccaaccagatggctaggagattcacctatttagcatgatatcttttattgataaaaaaatataaaacttccattaaatttttaagctactacaatcctattaaattttaacttaccagtgttctcaatgctacataatttaaaatcattgaaatcttctgattttaactcctcagtcttgaaatctacttatttttagttacatatatatccaatctactgccgctagtagaagaagcttggaatttgagaaaaaaatcagacgttttgtatattctcatattcactaatttattttttaaatgagtttctgcaatgcatcaagcagtggcaaaacaggagaaaaattaaaattggttgaaaagatatgtgtgccaaacaatcccttgaaatttgatgaagtgactaatcctgagttattgtttcaaatgtgtacctgtttatacaagggtatcacctttgaaatctcaacattaaatgaaattttataagcaatttgttgtaacatgattattataaaattctgatataacattttttattacctgtttagagtttaaagagagaaaaggagttaagaataattacattttcattagcattgtccgggtgcaaaaacttctaacactatcttcaaatctttttctccattgccttctgaacatacccacttgggtatctcattagcactgcaaattcaacattttcgattgctaatttttctccctaaatatttatttgttttctcagctttagccaatgtttcactattgaccatttgctcaagtatagtgacgcttcaatgaccttcagagagctgtttcagtccttcctggactacttgcatgcttccaacaaaatgaagcactcttgatgtcagtcactcaaataaatggaaatgggcccatttactaggaatgttaacagaataaaaagatagacgtgacaccagttgcttcagtccatctccatttacttgcttaaggcctggccatatttctcacagttgatatggcgcagggcacatgtttaaatggctgttcttgtaggatggtttgactgttggattcctcatcttccctctccttaggaaggaaggttacagtagtactgttggctcctggaatatagattcataaagaactaatggagtatcatctcccactgctcttgt

SEQ ID NO: 59 - DNA, 866 bp, Homo sapiens
  misc_feature: sequence of STAR59
gagatcacgccactgcactccagcctgggggacagagcaagactccatctcagaaacaaacaaacacacaaagccagtcaaggtgtttaattcgacggtgtcaggctcaggtctcttgacaggatacatccagcacccgggggaaacgtcgatgggtggggtggaatctattttgtggcctcaagggagggtttgagaggtagtcccgcaagcggtgatggcctaaggaagcccctccgcccaagaagcgatattcatttctagcctgtagccacccaagagggagaatcgggctcgccacagaccccacaacccccaacccaccccacccccacccctcccacctcgtgaaatgggctctcgctccgtcaggctctagtcacaccgtgtggttttggaacctccagcgtgtgtgcgtgggttgcgtggtggggtggggccggctgtggacagaggaggggataaagcggcggtgtcccgcgggtgcccgggacgtggggcgtggggcgtgggtggggtggccagagccttgggaactcgtcgcctgtcgggacgtctcccctcctggtcccctctctgacctacgctccacatcttcgccgttcagtggggaccttgtgggtggaagtcaccatccctttggactttagccgacgaaggccgggctcccaagagtctccccggaggcggggccttgggcaggctcacaaggatgctgacggtgacggttggtgacggtgatgtacttcggaggcctcgggccaatgcagaggtatccatttgacctcggtgggacaggtcagctttgcggagtcccgtgcgtccttccagagactcatccagcgctagcaagcatggtcccgagg

SEQ ID NO: 60 - DNA, 2067 bp, Homo sapiens
  misc_feature: sequence of STAR60
agcagtgcagaactggggaagaagaagagtccctacaccacttaatactcaaaagtactcgcaaaaaataacacccctcaccaggtggcatnattactctccttcattgagaaaattaggaaactggacttcgtagaagctaattgctttatccagagccacctgcatacaaacctgcagcgccacctgcatacaaacctgtcagccgaccccaaagccctcagtcgcaccaagcctctgctgcacaccctcgtgccttcacactggccgttccccaagcctggggcatactncccagctctgagaaatgtattcatccttcaaagccctgctcatgtgtcctnntcaacaggaaaatctcccatgagatgctctgctatccccatctctcctgccccatagcttaggcanacttctgtggtggtgagtcctgggctgtgctgtgatgtgttcgcctgcnatgtntgttcttccccacaatgatgggcccctgaattctctatctctagcacctgtgctcagtaaaggcttgggaaaccaggctcaaagcctggcccagatgccaccttttccagggtgcttccgggggccaccaaccagagtgcagccttctcctccaccaggaactcttgcagccccacccctgagcacctgcaccccattacccatctttgtttctccgtgtgatcgtattattacagaattatatactgtattcttaatacagtatataattgtataattattcttaatacagtatataattatacaaatacaaaatatgtgttaatggaccgtttatgttactggtaaagctttaagtcaacagtgggacattagttaggtttttggcgaagtcaaaagttatatgtgcattttcaacttcttgaggggtcggtacntctnacccccatgttgttcaanggtcaactgtctacacatatcatagctaattcactacagaaatgttagcttgtgtcactagtatctccccttctcataagcttaatacacataccttgagagagctcttggccatctctactaatgactgaagtttttatttattatagatgtcataataggcataaaactacattacatcattcgagtgccaattttgccaccttgaccctcttttgcaaaacaccaacgtcagtacacatatgaagaggaaactgcccgagaactgaagttcctgagaccaggagctgcaggcgttagatagaatatggtgacgagagttacgaggatgacgagagtaaatacttcatactcagtacgtgccaagcactgctataagcgctctgtatgtgtgaagtcatttaatcctcacagcatcccacggtgtaattattttcattatccccatgagggaacagaaactcagaacggttcaacacatatgcgagaagtcgcagccggtcagtgagagagcaggttcccgtccaagcagtcagaccccgagtgcacactctcgacccctgtccagcagactcactcgtcataaggcggggagtgntctgtttcagccagatgctttatgcatctcagagtacccaaaccatgaaagaatgaggcagtattcangagcagatggngctgggcagtaaggctgggcttcagaatagctggaaagctcaagtnatgggacctgcaagaaaaatccattgtttngataaatagccaaagtccctaggctgtaaggggaaggtgtgccaggtgcaagtggagctctaatgtaaaatcgcacctgagtctcctggtcttatgagtnctgggtgtaccccagtgaaaggtcctgctgccaccaagtgggccatggttcagctgtgtaagtgctgagcggcagccggaccgcttcctctaacttcacctccaaaggcacagtgcacctggttcctccagcactcagctgcgaggcccctagccagggtcccggcccccggcccccggcagctgctccagcttccttccccacagcattcaggatggtctgcgttcatgtagacctttgttttcagtctgtgctccgaggtcactggcagcactagccccggctcctgt

SEQ ID NO: 61 - DNA, 1470 bp, Homo sapiens
  misc_feature: sequence of STAR61
cagcccccacatgcccagccctgtgctcagctctgcagcggggcatggtgggcagagacacagaggccaaggccctgcttcggggacggtgggcctgggatgagcatggccttggccttcgccgagagtnctcttgtgaaggaggggtcaggaggggctgctgcagctggggaggagggcgatggcactgtggcangaagtgaantagtgtgggtgcctngcaccccaggcacggccagcctggggtatggacccggggccntctgttctagagcaggaaggtatggtgaggacctcaaaaggacagccactggagagctccaggcagaggnacttgagaggccctggggccatcctgtctcttttctgggtctgtgtgctctgggcctgggcccttcctctgctcccccgggcttggagagggctggccttgcctcgtgcaaaggaccactctagactggtaccaagtctggcccatggcctcctgtgggtgcaggcctgtgcgggtgacctgagagccagggctggcaggtcagagtcaggagagggatggcagtggatgccctgtgcaggatctgcctaatcatggtgaggctggaggaatccaaagtgggcatgcactctgcactcatttctttattcatgtgtgcccatcccaacaagcagggagcctggccaggagggcccctgggagaaggcactgatgggctgtgttccatttaggaaggatggacggttgtgagacgggtaagtcagaacgggctgcccacctcggccgagagggccccgtggtgggttggcaccatctgggcctggagagctgctcaggaggctctctagggctgggtgaccaggnctggggtacagtagccatgggagcaggtgcttacctggggctgtccctgagcaggggctgcattgggtgctctgtgagcacacacttctctattcacctgagtcccnctgagtgatgagnacacccttgttttgcagatgaatctgagcatggagatgttaagtggcttgcctgagccacacagcagatggatggtgtagctgggacctgagggcaggcagtcccagcccgaggacttcccaaggttgtggcaaactctgacagcatgaccccagggaacacccatctcagctctggtcagacactgcggagttgtgttgtaacccacacagctggagacagccaccctagccccacccttatcctctcccaaaggaacctgccctttcccttcattttcctcttactgcattgagggaccacacagtgtggcagaaggaacatgggttcaggacccagatggacttgcttcacagtgcagccctcctgtcctcttgcagagtgcgtcttccactgtgaagttgggacagtcacaccaactcaatactgctgggcccgtcacacggtgggcaggcaacggatggcagtcactggctgtgggtctgcagaggtgg

SEQ ID NO: 62 - DNA, 1011 bp, Homo sapiens
  misc_feature: sequence of STAR62
agtgtcaaatagatctacacaaaacaagataatgtctgcccatttttccaaagataatgtggtgaagtgggtagagagaaatgcatccattctccccacccaacctctgctaaattgtccatgtcacagtactgagaccagggggcttattcccagcgggcagaatgtgcaccaagcacctcttgtctcaatttgcagtctaggccctgctatttgatggtgtgaaggcttgcacctggcatggaaggtccgttttgtacttcttgctttagcagttcaaagagcagggagagctgcgagggcctctgcagcttcagatggatgtggtcagcttgttggaggcgccttctgtggtccattatctccagcccccctgcggtgttgctgtttgcttggcttgtctggctctccatgccttgttggctccaaaatgtcatcatgctgcaccccaggaagaatgtgcaggcccatctcttttatgtgctttgggctattttgattccccgttgggtatattccctaggtaagacccagaagacacaggaggtagttgctttgggagagtttggacctatgggtatgaggtaatagacacagtatcttctctttcatttggtgagactgttagctctggccgcggactgaattccacacagctcacttgggaaaactttattccaaaacatagtcacattgaacattgtggagaatgagggacagagaagaggccctagatttgtacatctgggtgttatgtctataaatagaatgctttggtggtcaactagacttgttcatgttgacatttagtcttgccttttcggtggtgatttaaaaattatgtatatcttgtttggaatatagtggagctatggtgtggcattttcatctggctttttgtttagctcagcccgtcctgttatgggcagccttgaagctcagtagctaatgaagaggtatcctcactccctccagagagcggtcccctcacggctcattgagagtttgtca

SEQ ID NO: 63 - DNA, 1410 bp, Homo sapiens
  misc_feature: sequence of STAR63
ccacagcctgatcgtgctgtcgatgagaggaatctgctctaagggtctgagcggagggagatgccgaagctttgagctttttgtttctggcttaaccttggtggattttcaccctctgggcattacctcttgtccaggggaggggctgggggagtgcctggagctgtagggacagagggctgagtgggggggactgcttgggctgaccacataatattctgctgcgtattaatttttttttgagacagtctttctctgttgcccaggctggagtgtaatggcttgatagctcactgccacctccgcctcctgggttcaagtgattctcctgcttcagcttccggagtagctgggactgcaggtgcccgccaccatggctggctaatttttgtatttttattagcaatggggttttgctatgttgcccaggccggtcccgaactcctgccctcaagtgatacacctgcctcggcctcccaaagtgctgggattagaggcttgagccactgcgcctggccagctgcatattgttaattagacataaaatgcaaaataagatgatataaacacaaaggtgtgaaataagatggacacctgctgagcgcgcctgtcctgaagcatcgcccctctgcaaaagcaggggtcagcatgtgttctccggtccttgctcttacagaggagtgagctgcctatgcgtcttccagccacttcctgggctgctcagaggcctctcacgggtgttctgggttgctgccacttgcaggggtgctgaggcggggctcctcccgtgcggggcatgtccaggccgccctctctgaaggcttggcaggtacaggtgggagtgggggtctctgggctgctgtggggactgggcaggctcctggaagacctccctgtgtttgggctgaaagcgcagcccgaggggaggtccccagggaggccgctgtcgggggtgggggcttggaggagggaggggccgaggagccggcgacactccgtgacggcccaggaacgtccctaaacaaggcgccgcgttctcgatggggtggggtccgctttcttttctcaaaagctgcagttactccatgctcggaggactggcgtccgcgccctgttccaatgctgccccggggccctggccttggggaatcggggccttggactggaccctgggggcttcgcggagccgggcctggcggggcgagcggagcagaggctgggcagccccggggaagcgctcgccaaagccgggcgctgctcccagagcgcgaggtgcagaaccagaggctggtcccgcggcgctaacgagagaagaggaagcgcgctgtgtagagggcgcccaccccgtggggcgaacccccttcctcaactccatggacggggctcatgggttcccagcggctcagacgc

SEQ ID NO: 64 - DNA, 1414 bp, Homo sapiens
  misc_feature: sequence of STAR64
tggatcagatttgttttataccctcccttctactgctctgagagttgtacatcacagtctactgtatctgtttcccattattataatttttttgcactgtgcttgcctgaagggagcctcaagttcatgagtctccctaccctcctcccaaatgagacatggacctttgaatgctttcctgggaccaccaccccacctttcatgctgctgttatccaggattttagttcaacagtgttttaaccccccaaatgagtcatttttattgtttcgtatagtgaatgtgtatttgggtttgcttatatggtgacctgtttatttgctcctcattgtacctcatgctctgctctttccttctagattcagtctctttcctaatgaggtgtctcgcagcaattctttacaagacagccaagataggccagctctcagagcacttgttgtctgaaaaagtcttgtcttatttaatttctttttcttagagatggggtctcattatgttacccacactggtctcaaacttctggcttaaagcggtcctcccaccttggcctcccaaagtgctaggattacaggcgtgagcgacctcgtccagcctgtctgagaaagcgtttgttttgcccttgctctcagatgacagtttggggatagaattctaggtggacggtttttttccttcagccctttgaagagtctgtattttcattatctccctgcattagatgttcttttgcaagtaacgtgtcttttctctctgggtattcttaaggttttctctttgcctttggtgagctgcagtggatttgcttttttcaagaggtcaagagaaaggaaagtgtgaggtttctgttttttactgacaatttgtttgttgatttgttttcccacccagaggttccttgccactttgccaggctggaaggcagacttcttctggtgtcctgttcacagacggggcagcctgcggaaggccctgccacatgcagggcctcggtcctcattcccttgcatgtggacccgggcgtgactcctgttcaggctggcacttcccagagctgagccccagcctgaccttcctcccatactgtcttcacaccccctcctttcttctgatacctggaggttttcctttctttcctgtcacctccacttggattttaaatcctctgtctgtggaattgtattcggcacaggaagatgcttgcaagggccaggctcatcagccctgtccctgctgctggaagcagcacagcagagcctcatgctcaggctgagatggagcagaggcctgcagacgagcacccagctcagctggggttggcgccgatggtggagggtcctcgaaagctctggggacgatggcagagctattggcaggggagccgcagggtcttttgagcccttaaaagatctct

SEQ ID NO: 65 - DNA, 1310 bp, Homo sapiens
  misc_feature: sequence of STAR65
gtgaatgttgatggatcaaatatctttctgtgttgtttatcaaagttaaaataaatgtggtcatttaaaggacaaaagatgaggggttggagtctgttcaagcaaagggtatattaggagaaaagcagaattctctccctgtgaagggacagtgactcctattttccacctcatttttactaactctcctaactatctgcttaggtagagatatatccatgtacatttataaaccacagtgaatcatttgattttggaataaagatagtataaaatgtgtcccagtgttgatatacatcatacattaaatatgtctggcagtgttctaattttacagttgtccaaagataatgttagggcatactggctatggatgaagctccaatgttcagattgcaaagaaacttagaattttactaatgaaaccaaatacatcccaagaaatttttcagaagaaaaaaagagaaactagtagcaaagtaaagaatcaccacaatatcatcagattttttttatatgtagaatatttattcagttcttttttcaagtacaccttgtcttcattcattgtactttattttttgtgaaggtttaaatttatttcttctatgtgtttagtgatatttaaaatttttatttaatcaagtttatcagaaagttctgttagaaaatatgacgaggctttaattccgccatctatattttccgctattatataaagataattgttttctctttttaaaacaacttgaattgggattttatatcataattttttaatgtctttttttattatactttaagttctgggatacatgtgcagaacgtgcaggtgtgttacatagatatacacgtgccatggtggtttgctgcacccactaacctgttatcgacattaggtatttctcctaatgctatcaccccctatttccccaccccccgagaggccccagtgtgtgatgttctcctccctgtgtccatgtgttctcattgttcatctcccacttatggtatctaccataaccttgaaattgtcttatgcattcacttgtttggttgttatatagcctccatcaggacagggatatttgctgctgcttcttttttttttctttttgagacagtcttgctccgtcatccaggctggagtgcttctcggctcaatgcaacctccacctcccaggtttaagcgattctccaacttcagcctcccaaatggctgggactgcaggcatgcaccactacacctggctaatttttgtatttgtaatagagacaatgtttcaccatgttggccaggctggtctcga

SEQ ID NO: 66 - DNA, 1917 bp, Homo sapiens
  misc_feature: sequence of STAR67
aggatcctaaaattttgtgaccctagagcaagtactaactatgaaagtgaaatagagaatgaaggaattatttaattaagtccagcaaaacccaaccaaatcatctgtaaaatatatttgttttcaacatccaggtattttctgtgtaaaaggttgagttgtatgctgacttattgggaaaaataattgagttttccccttcactttgccagtgagaggaaatcagtactgtaattgttaaaggttacccatacctacctctactaccgtctagcataggtaaagtaatgtacactgtgaagtttcctgcttgactgtaatgttttcagtttcatcccattgattcaacagctatttattcagcacttactacaaccatgctggaaacccaagagtaaataggctgtgttactcaacaggactgaggtacagccgaactgtcaggcaaggttgctgtcctttggacttgcctgctttctctctatgtaggaagaagaaatggacataccgtccaggaaatagatatatgttacatttccttattccataattaatattaataaccctggacagaaactaccaagtttctagacccttatagtaccaccttaccctttctggatgaatccttcacatgttgatacattttatccaaatgaaaattttggtactgtaggtataacagacaaagagagaacagaaaactagagatgaagtttgggaaaaggtcaagaaagtaaataatgcttctagaagacacaaaaagaaaaatgaaatggtaatgttgggaaagttttaatacattttgccctaaggaaaaaaactacttgttgaaattctacttaagactggaccttttctctaaaaattgtgcttgatgtgaattaaagcaacacagggaaatttatgggctccttctaagttctacccaactcaccgcaaaactgttcctagtaggtgtggtatactctttcagattctttgtgtgtatgtatatgtgtgtgtgtgtgtgtgtttgtatgtgtacagtctatatacatatgtgtacctacatgtgtgtatatataaatatatatttacctggatgaaatagcatattatagaatattcttttttctttaaatatatatgtgcatacatatgtatatgcacatatatacataaatgtagatatagctaggtaggcattcatgtgaaacaaagaagcctattactttttaatggttgcatgatattccatcataggagtatagtacaacttatgtaacacacatttggcttgttgtaaaattttggtattaataaaatagcacatatcatgcaaagacacccttgcataggtctattcattctttgatttttaccttaggacaaaatttaaaagtagaatttctgggtcaagcagtatgctcatttaaaatgtcattgcatatttccaaattgtcctccagaaaagtagtaacagtaacaattgatggactgcgtgttttctaaaacttgcatttttttccttattggtgaggtttggcattttccatatgtttattggcattttaattttttttggttcatgtcttttattcccttcctgcaaatttgtggtgtgtctcaactttatttatactctcattttcataattttctaaaggaatttgactttaaaaaaataagacagccaatgctttggtttaatttcattgctgctttttgaagtgactgctgtgtttttatatacttttatattttgttgttttagcaaattcttctatattataattgtgtatgctggaacaaaaagttatatttcttaatctagataaaatatttcaagatgttgtaattacagtcccctctaaaatcatataaatagacgcatagctgtgtgatttgtaattagttatgtccattgatagatcc

SEQ ID NO: 67 - DNA, 11 bp, Artificial
  sequence around startcodon of wild-type zeocin resistance gene
aaaccatggcc

SEQ ID NO: 68 - DNA, 50 bp, Artificial
  primer ZEOforwardMUT
gatctcgcgatacaggatttatgttggccaagttgaccagtgccgttccg

SEQ ID NO: 69 - DNA, 26 bp, Artificial
  primer ZEO-WTreverse
aggcgaattcagtcctgctcctcggc

SEQ ID NO: 70 - DNA, 45 bp, Artificial
  primer ZEO-LEUreverse
aggccccgcccccacggctgctcgccgatctcggtcaaggccggc

SEQ ID NO: 71 - DNA, 45 bp, Artificial
  primer ZEO-THRreverse
aggccccgcccccacggctgctcgccgatctcggtggtggccggc

SEQ ID NO: 72 - DNA, 44 bp, Artificial
  primer ZEO-VALreverse
aggccccgcccccacggctgctcgccgatctcggtccacgccgg

SEQ ID NO: 73 - DNA, 11 bp, Artificial
  sequence around startcodon of wt d2EGFP
gaattcatggg

SEQ ID NO: 74 - DNA, 49 bp, Artificial
  primer d2EGFPforwardBamHI
gatcggatcctatgaggaattcgccaccatggtgagcaagggcgaggag

SEQ ID NO: 75 - DNA, 41 bp, Artificial
  primer d2EGFPreverseNotI
aaggaaaaaagcggccgcctacacattgatcctagcagaag

SEQ ID NO: 76 - DNA, 57 bp, Artificial
  spacer sequence
tcgatccaaagactgccaaatctagatccgagattttcaggagctaaggaagctaaa

SEQ ID NO: 77 - DNA, 99 bp, Artificial
  primer ZEOforwardBamHI-ATGmut/space
gatcggatccttggtttatgtcgatccaaagactgccaaatctagatccgagattttcaggagctaaggaagctaaagccaagttgaccagtgaagttc

SEQ ID NO: 78 - DNA, 38 bp, Artificial
  primer ZEOforwardBamHI-GTG
gatcggatccaccgtggccaagttgaccagtgccgttc

SEQ ID NO: 79 - DNA, 38 bp, Artificial
  primer ZEOforwardBamHI-TTG
gatcggatccaccttggccaagttgaccagtgccgttc

SEQ ID NO: 80 - DNA, 35 bp, Artificial
  primer BSDBamHIforward
gatcggatccaccatggccaagcctttgtctcaag

SEQ ID NO: 81 - DNA, 25 bp, Artificial
  primer BSD150reverse
gtaaaatgatatacgttgacaccag

SEQ ID NO: 82 - DNA, 25 bp, Artificial
  primer BSD150forward
ctggtgtcaacgtatatcattttac

SEQ ID NO: 83 - DNA, 24 bp, Artificial
  primer BSD250reverse
gccctgttctcgtttccgatcgcg

SEQ ID NO: 84 - DNA, 24 bp, Artificial
  primer BSD250forward
cgcgatcggaaacgagaacagggc

SEQ ID NO: 85 - DNA, 24 bp, Artificial
  primer BSD350reverse
gccgtcggctgtccgtcactgtcc

SEQ ID NO: 86 - DNA, 24 bp, Artificial
  primer BSD350forward
ggacagtgacggacagccgacggc

SEQ ID NO: 87 - DNA, 38 bp, Artificial
  primer BSD399reverse
gatcgaattcttagccctcccacacgtaaccagagggc

SEQ ID NO: 88 - DNA, 103 bp, Artificial
  primer BSDforwardBamHIAvrII-ATGmut/space
gatcggatcctaggttggtttatgtcgatccaaagactgccaaatctagatccgagattttcaggagctaaggaagctaaagccaagcctttgtctcaagaag

SEQ ID NO: 89 - DNA, 44 bp, Artificial
  primer BSD399reverseEcoRIAvrII
gatcgaattccctaggttagccctcccacacgtaaccagagggc

SEQ ID NO: 90 - DNA, 42 bp, Artificial
  primer BSDforwardBamHIAvrII-GTG
gatcggatcctaggaccgtggccaagcctttgtctcaagaag

SEQ ID NO: 91 - DNA, 42 bp, Artificial
  primer BSDforwardBamHIAvrII-TTG
gatcggatcctaggaccttggccaagcctttgtctcaagaag

SEQ ID NO: 92 - DNA, 375 bp, Artificial
  wt zeocin resistance gene
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 93 - protein, 124 aa, Artificial
  Synthetic Construct
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 94 - DNA, 399 bp, Artificial
  wt blasticidin resistance gene
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 95 - protein, 132 aa, Artificial
  Synthetic Construct
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 96 - DNA, 600 bp, Artificial
  wt puromycin resistance gene
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctga

SEQ ID NO: 97 - protein, 199 aa, Artificial
  Synthetic Construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVECPKDRATWCMTRKPGA

SEQ ID NO: 98 - DNA, 564 bp, Artificial
  wt DHFR gene (from mouse)
atggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaa

SEQ ID NO: 99 - protein, 187 aa, Artificial
  Synthetic Construct
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 100 - DNA, 1143 bp, Artificial
  wt hygromycin resistance gene
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggaggcaaaggaattcgggagatgggggaggctaactgaaacacggaaggagacaataccggaaggaacccgcgctatgacggcaataaaaagacagaataaaacgcacgggtgttgggtcgtttgttcataa

SEQ ID NO: 101 - protein, 380 aa, Artificial
  Synthetic Construct
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPEAKEFGRWGRLTETRKETIPEGTRAMTAIKRQNKTHGCWVVCS

SEQ ID NO: 102 - DNA, 804 bp, Artificial
  wt neomycin resistance gene
atgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 103 - protein, 267 aa, Artificial
  Synthetic Construct
MGSAIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 104 - DNA, 1121 bp, Artificial
  wt glutamine synthase gene (human)
atgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctccagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctggtggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaatta

SEQ ID NO: 105 - protein, 373 aa, Artificial
  Synthetic Construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSGGVANRSASIRIPRTVGQEKKGYFEDRRPSANCDPFSVTEALIRTCLLNETGDEPFQYKN

SEQ ID NO: 106 - DNA, 43 bp, Artificial
  primer GTGspaceBamHIF
gaattcggatccaccgtggcgatccaaagactgccaaatctag

SEQ ID NO: 107 - DNA, 42 bp, Artificial
  primer ZEOTTTGTGBamHIF
gaattcggatcctttgtggccaagttgaccagtgccgttccg

SEQ ID NO: 108 - DNA, 46 bp, Artificial
  primer ZEOForwardGTG-Thr9
aattggatccaccgtggccaagttgaccagtgccgttaccgtgctc

SEQ ID NO: 109 - DNA, 46 bp, Artificial
  pimer ZEOForward GTG-Phe9
aattggatccaccgtggccaagttgaccagtgccgttttcgtgctc

SEQ ID NO: 110 - DNA, 43 bp, Artificial
  primer TTGspaceBamHIF
gaattcggatccaccttggcgatccaaagactgccaaatctag

SEQ ID NO: 111 - DNA, 46 bp, Artificial
  primer ZEOForwardTTG-Thr9
aattggatccaccttggccaagttgaccagtgccgttaccgtgctc

SEQ ID NO: 112 - DNA, 46 bp, Artificial
  pimer ZEOForwardTTG-Phe9
aattggatccaccttggccaagttgaccagtgccgttttcgtgctc

SEQ ID NO: 113 - DNA, 37 bp, Artificial
  primer PURO BamHI F
gatcggatccatggttaccgagtacaagcccacggtg

SEQ ID NO: 114 - DNA, 35 bp, Artificial
  primer PURO300 R LEU
cagccgggaaccgctcaactcggccaggcgcgggc

SEQ ID NO: 115 - DNA, 49 bp, Artificial
  primer PURO300FLEU
cgagttgagcggttcccggctggccgcgcagcaacagctggaaggcctc

SEQ ID NO: 116 - DNA, 44 bp, Artificial
  primer PURO600RLEU
aagcttgaattcaggcaccgggcttgcgggtcaggcaccaggtc

SEQ ID NO: 117 - DNA, 42 bp, Artificial
  primer PUROBamHI TTG1F
gaattcggatccaccttggttaccgagtacaagcccacggtg

SEQ ID NO: 118 - DNA, 804 bp, Artificial
  modified neomycin resistance gene lacking internal ATG sequences
atgggatcggccattgaacaagacggattgcacgcaggttctccggccgcttgggtggagaggctattcggctacgactgggcacaacagacaatcggctgctctgacgccgccgtgttccggctgtcagcgcaggggcgcccggttctttttgtcaagaccgacctgtccggtgccctgaacgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcctggctgacgcactgcggcggctgcatacgcttgatccggctacctgcccattcgaccaccaagcgaaacatcgcatcgagcgagcacgtactcggctggaagccggtcttgtcgatcaggacgatctggacgaagagcatcaggggctcgcgccagccgaactgttcgccaggctcaaggcgcgcctgcccgacggcgacgatctcgtcgtgacccacggcgacgcctgcttgccgaatatcctggtggaaaacggccgcttttctggattcatcgactgtggccggctgggtgtggcggaccgctatcaggacatagcgttggctacccgtgatattgctgaagagcttggcggcgagtgggctgaccgcttcctcgtgctttacggtatcgccgctcccgattcgcagcgcatcgccttctatcgccttcttgacgagttcttctga

SEQ ID NO: 119 - protein, 267 aa, Artificial
  Synthetic Construct
MGSAIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSILADALRRLHTLDPATCPFDHQAKHRIERARTRLEAGLVDQDDLDEEHQGLAPAELFARLKARLPDGDDLVVTHGDACLPNILVENGRFSGFIDCGRLGVADRYQDIALATRDIAEELGGEWADRFLVLYGIAAPDSQRIAFYRLLDEFF

SEQ ID NO: 120 - DNA, 40 bp, Artificial
  primer NEO-F-HindIII
gatcaagcttttggatcggccattgaaacaagacggattg

SEQ ID NO: 121 - DNA, 36 bp, Artificial
  primer NEO EcoRI 800R
aagcttgaattctcagaagaactcgtcaagaaggcg

SEQ ID NO: 122 - DNA, 564 bp, Artificial
  modified dhfr gene lacking internal ATG sequences
atggttcgaccattgaactgcatcgtcgccgtgtcccaaaatctggggattggcaagaacggagacctaccctggcctccgctcaggaacgagttcaagtacttccaaagactgaccacaacctcttcagtggaaggtaaacagaatctggtgattctgggtaggaaaacctggttctccattcctgagaagaatcgacctttaaaggacagaattaatatagttctcagtagagaactcaaagaaccaccacgaggagctcattttcttgccaaaagtttggacgacgccttaagacttattgaacaaccggaattggcaagtaaagtagacctggtttggatagtcggaggcagttctgtttaccaggaagccctgaatcaaccaggccacctcagactctttgtgacaaggattctgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaa

SEQ ID NO: 123 - protein, 187 aa, Artificial
  Synthetic Construct
MVRPLNCIVAVSQNLGIGKNGDLPWPPLRNEFKYFQRLTTTSSVEGKQNLVILGRKTWFSIPEKNRPLKDRINIVLSRELKEPPRGAHFLAKSLDDALRLIEQPELASKVDLVWIVGGSSVYQEALNQPGHLRLFVTRILQEFESDTFFPEIDLGKYKLLPEYPGVLSEVQEEKGIKYKFEVYEKKD

SEQ ID NO: 124 - DNA, 36 bp, Artificial
  primer DHFR-F-HindIII
gatcaagcttttgttcgaccattgaactgcatcgtc

SEQ ID NO: 125 - DNA, 36 bp, Artificial
  primer DHFR-EcoRI-600-R
aagcttgaattcttagtctttcttctcgtagacttc

SEQ ID NO: 126 - DNA, 154 bp, Artificial
  combined synthetic polyadenylation sequence and pausing signal from the human alpha2 globin gene
aataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 127 - DNA, 596 bp, Artificial
  IRES sequence
gcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccccgggata

(19) United States
(12) Patent Application Publication
(10) Pub. No.: US 2006/0141577 A1
(43) Pub. Date: Jun. 29, 2006

(54) Selection of host cells expressing protein at high levels

(76) Inventors: Arie Pieter Otte, Amersfoort (NL); Henricus Johannes Maria Van Blokland, Wijdewormer (NL); Theodorus Hendrikus Jacobus Kwaks, Amsterdam (NL); Richard George Antonius Bernards Sewalt, Arnhem (NL)

Correspondence Address: TRASK BRITT, P.O. BOX 2550, SALT LAKE CITY, UT, 84110, US

(21) Appl. No.: 11/359,953

(22) Filed: Feb. 21, 2006

Related U.S. Application Data
(63) Continuation-in-part of application No. 11/269,525, filed on Nov. 7, 2005.
(60) Provisional application No. 60/626,301, filed on Nov. 8, 2004.
(60) Provisional application No. 60/696,610, filed on Jul. 5, 2005.

(30) Foreign Application Priority Data
Nov. 8, 2004 (EP) 04105593.0

(51) Int. Cl. C12P 21/06; C07H 21/04; C07K 14/705; C07K 16/28

(52) U.S. Cl. 435/69.1; 435/455; 435/325; 530/350; 530/388.22; 536/23.5

(57) ABSTRACT

The invention provides a DNA molecule comprising a multicistronic transcription unit coding for i) a polypeptide of interest, and for ii) a selectable marker polypeptide functional in a eukaryotic host cell, wherein the polypeptide of interest has a translation initiation sequence separate from that of the selectable marker polypeptide, and wherein the coding sequence for the polypeptide of interest is upstream from the coding sequence for the selectable marker polypeptide in said multicistronic transcription unit, and wherein an internal ribosome entry site (IRES) is present downstream from the coding sequence for the polypeptide of interest and upstream from the coding sequence for the selectable marker polypeptide, and wherein the nucleic acid sequence coding for the selectable marker polypeptide in the coding strand comprises a GTG or a TTG startcodon. The invention also provides methods for obtaining host cells expressing a polypeptide of interest, said host cells comprising the DNA molecules of the invention. The invention further provides the production of polypeptides of interest, comprising culturing host cells comprising the DNA molecules according to the invention.

### CROSS-REFERENCE TO RELATED APPLICATIONS

This application is a continuation-in-part of co-pending U.S. patent application Ser. No. 11/269,525, filed Nov. 7, 2005, the contents of the entirety of which is incorporated by this reference, which application claims priority under 35 U.S.C. Section 119(e) to U.S. Provisional Patent Application Ser. No. 60/626,301, filed Nov. 8, 2004, and to U.S. Provisional Patent Application Ser. No. 60/696,610, filed Jul. 5, 2005, the contents of the entirety of both of which are incorporated by this reference. The U.S. patent application Ser. No. 11/269,525 also claims the benefit of EP 04105593.0, filed Nov. 8, 2004.

### STATEMENT ACCORDING TO 37 C.F.R. § 1.52(e)(5)—SEQUENCE LISTING SUBMITTED ON COMPACT DISC

Pursuant to 37 C.F.R. § 1.52(e)(1)(ii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “2578-7691US seq list.txt” which is 186 KB and created on Feb. 21, 2006.

### BACKGROUND OF THE INVENTION

### Field of the Invention

The invention relates to the field of molecular biology and biotechnology. More specifically the present invention relates to means and methods for improving the selection of host cells that express proteins at high levels.

Proteins can be produced in various host cells for a wide range of applications in biology and biotechnology, for instance as biopharmaceuticals. Eukaryotic and particularly mammalian host cells are preferred for this purpose for expression of many proteins, for instance when such proteins have certain posttranslational modifications such as glycosylation. Methods for such production are well established, and generally entail the expression in a host cell of a nucleic acid (also referred to as ‘transgene’) encoding the protein of interest. In general, the transgene together with a selectable marker gene is introduced into a precursor cell, cells are selected for the expression of the selectable marker gene, and one or more clones that express the protein of interest at high levels are identified, and used for the expression of the protein of interest.

One problem associated with the expression of transgenes is that it is unpredictable, stemming from the high likelihood that the transgene will become inactive due to gene silencing (McBurney et al., 2002), and therefore many host cell clones have to be tested for high expression of the transgene.

Methods to select recombinant host cells expressing relatively high levels of desired proteins are known.

One method describes the use of selectable marker proteins with mutations in their coding sequence that diminish, but not destroy the function of the marker (e.g., WO 01/32901). The rationale is that higher levels of the mutant marker expression are required when selection conditions are employed and therefore selection for high expression of the marker is achieved, therewith concomitantly selecting host cells that also express the gene of interest at high levels.

Another method makes use of a selection marker gene under control of a promoter sequence that has been mutated such that the promoter has an activity level substantially below that of its corresponding wild type (U.S. Pat. No. 5,627,033).

Another method describes the use of an impaired dominant selectable marker sequence, such as neomycin phosphotransferase with an impaired consensus Kozak sequence, to decrease the number of colonies to be screened and to increase the expression levels of a gene of interest that is co-linked to the dominant selectable marker (U.S. Pat. Nos. 5,648,267 and 5,733,779). In preferred embodiments therein, the gene of interest is placed within an (artificial) intron in the dominant selectable marker. The gene of interest and the dominant selectable marker are in different transcriptional cassettes and each contains its own eukaryotic promoter in this method (U.S. Pat. Nos. 5,648,267 and 5,733,779).

Another method uses the principle of a selectable marker gene containing an intron that does not naturally occur within the selectable gene, wherein the intron is capable of being spliced in a host cell to provide mRNA encoding a selectable protein and wherein the intron in the selectable gene reduces the level of selectable protein produced from the selectable gene in the host cell (European Patent 0724639 B1).

In yet another method, DNA constructs are used comprising a selectable gene positioned within an intron defined by a 5′ splice donor site comprising an efficient splice donor sequence such that the efficiency of splicing an mRNA having said splice donor site is between about 80-99%, and a 3′ splice acceptor site, and a product gene encoding a product of interest downstream of 3′ splice acceptor site, the selectable gene and the product gene being controlled by the same transcriptional regulatory region (U.S. Pat. No. 5,561,053).

In certain methods, use is made of polycistronic expression vector constructs. An early report of use of this principle describes a polycistronic expression vector, containing sequences coding for both the desired protein and a selectable protein, which coding sequences are governed by the same promoter and separated by a translational stop and start signal codons (U.S. Pat. No. 4,965,196). In preferred embodiments in U.S. Pat. No. 4,965,196, the selectable marker is the amplifiable DHFR gene. In a particularly preferred embodiment of the system described in U.S. Pat. No. 4,965,196, the sequence coding for the selectable marker is downstream from that coding for the desired polypeptide, such that procedures designed to select for the cells transformed by the selectable marker will also select for particularly enhanced production of the desired protein.

In further improvements based on the concept of multicistronic expression vectors, bicistronic vectors have been described for the rapid and efficient creation of stable mammalian cell lines that express recombinant protein. These vectors contain an internal ribosome entry site (IRES) between the upstream coding sequence for the protein of interest and the downstream coding sequence of the selection marker (Rees et al, 1996). Such vectors are commercially available, for instance the pIRES1 vectors from Clontech (CLONTECHniques, October 1996). Using such vectors for introduction into host cells, selection of sufficient expression of the downstream marker protein then automatically selects for high transcription levels of the multicistronic mRNA, and hence a strongly increased probability of high expression of the protein of interest is envisaged using such vectors.

Preferably in such methods, the IRES used is an IRES which gives a relatively low level of translation of the selection marker gene, to further improve the chances of selecting for host cells with a high expression level of the protein of interest by selecting for expression of the selection marker protein (see e.g. international publication WO 03/106684).

The present invention aims at providing improved means and methods for selection of host cells expressing high levels of proteins of interest.

### BRIEF SUMMARY OF THE INVENTION

U.S. patent application Ser. No. 11/269,525 (hereinafter the '525 application) and International Patent Application No. PCT/EP2005/055794, both incorporated in their entirety by reference herein, disclose a concept for selecting host cells expressing high levels of polypeptides of interest, the concept referred to therein as ‘reciprocal interdependent translation’. In that concept, a multicistronic transcription unit is used wherein a sequence encoding a selectable marker polypeptide is upstream of a sequence encoding a polypeptide of interest, and wherein the translation of the selectable marker polypeptide is impaired by mutations therein, whereas translation of the polypeptide of interest is very high (see e.g. FIG. 2 herein for a schematic view). The present invention provides alternative means and methods for selecting host cells expressing high levels of polypeptide.

In one aspect, the invention provides a DNA molecule comprising a multicistronic transcription unit coding for i) a polypeptide of interest, and for ii) a selectable marker polypeptide functional in a eukaryotic host cell, wherein the polypeptide of interest has a translation initiation sequence separate from that of the selectable marker polypeptide, and wherein the coding sequence for the polypeptide of interest is upstream from the coding sequence for the selectable marker polypeptide in said multicistronic transcription unit, and wherein an internal ribosome entry site (IRES) is present downstream from the coding sequence for the polypeptide of interest and upstream from the coding sequence for the selectable marker polypeptide, and wherein the nucleic acid sequence coding for the selectable marker polypeptide in the coding strand comprises a translation start sequence chosen from the group consisting of: a) an ATG startcodon in a non-optimal context for translation initiation, comprising the sequence (C/T)(A/T/G)(A/T/G)ATG(A/T/C) wherein the startcodon is underlined; b) a GTG startcodon; c) a TTG startcodon; d) a CTG startcodon; e) a ATT startcodon; and f) a ACG startcodon.

In certain embodiments thereof, the translation start sequence in the coding strand for the selectable marker polypeptide comprises an ATG sequence defining a startcodon, said ATG sequence being in a non-optimal context for translation initiation. This results in a decreased use of this ATG as startcodon, when compared to an ATG startcodon in an optimal context.

In a preferred embodiment, the translation start sequence in the coding strand for the selectable marker polypeptide comprises a startcodon different from an ATG startcodon, such as one of GTG, TTG, CTG, ATT, or ACG sequence, the first two thereof being the most preferred. Such non-ATG startcodons preferably are flanked by sequences providing for relatively good recognition of the non-ATG sequences as startcodons, such that at least some ribosomes start translation from these startcodons, i.e. the translation start sequence preferably comprises the sequence ACC[non-ATG startcodon]G or GCC[non-ATG startcodon]G.

In preferred embodiments, the selectable marker protein provides resistance against lethal and/or growth-inhibitory effects of a selection agent, such as an antibiotic.

Preferably, the coding sequence of the polypeptide of interest comprises an optimal translation start sequence.

The invention further provides expression cassettes comprising a DNA molecule according to the invention, which expression cassettes further comprise a promoter upstream of the multicistronic expression unit and being functional in a eukaryotic host cell for initiation transcription of the multicistronic expression unit, and said expression cassettes further comprising a transcription termination sequence downstream of the multicistronic expression unit.

In preferred embodiments thereof, such expression cassettes further comprise at least one chromatin control element chosen from the group consisting of a matrix or scaffold attachment region (MAR/SAR), an insulator sequence, a ubiquitous chromatin opener element (UCOE), and an anti-repressor sequence. Anti-repressor sequences are most preferred in this aspect, and in preferred embodiments said anti-repressor sequences are chosen from the group consisting of: a) any one SEQ. ID. NO. 1 through SEQ. ID. NO. 66; b) fragments of any one of SEQ. ID. NO. 1 through SEQ. ID. NO. 66, wherein said fragments have anti-repressor activity; c) sequences that are at least 70% identical in nucleotide sequence to a) or b) wherein said sequences have anti-repressor activity; and d) the complement to any one of a) to c). In certain preferred embodiments, said anti-repressor sequences are chosen from the group consisting of: STAR67 (SEQ. ID. NO. 66), STAR7 (SEQ. ID. NO. 7), STAR9 (SEQ. ID. NO. 9), STAR17 (SEQ. ID. NO. 17), STAR27 (SEQ. ID. NO. 27), STAR29 (SEQ. ID. NO. 29), STAR43 (SEQ. ID. NO. 43), STAR44 (SEQ. ID. NO. 44), STAR45 (SEQ. ID. NO. 45), STAR47 (SEQ. ID. NO. 47), STAR61 (SEQ. ID. NO. 61), and functional fragments or derivatives of these STAR sequences. In certain embodiments, the expression cassette comprises STAR67, or a functional fragment or derivative thereof, positioned upstream of the promoter driving expression of the multicistronic gene. In certain embodiments, the multicistronic gene is flanked on both sides by at least one anti-repressor sequence. In certain preferred embodiments, expression cassettes are provided according to the invention, comprising in 5′ to 3′ order: anti-repressor sequence A—anti-repressor sequence B—[promoter—multicistronic transcription unit according to the invention (encoding the functional selectable marker protein and downstream thereof the polypeptide of interest)—transcription termination sequence]—anti-repressor sequence C, wherein A, B and C may be the same or different.

In certain embodiments, the polypeptide of interest is a part of a multimeric protein, for example a heavy or light chain of an immunoglobulin.

The invention also provides DNA molecules comprising a sequence encoding a functional selectable marker polypeptide, characterized in that such DNA molecules comprise a mutation that decreases the translation initiation efficiency of the functional selectable marker polypeptide in a eukaryotic host cell. Preferably, such a DNA molecule comprises a GTG or a TTG startcodon followed by an otherwise functional selectable marker coding sequence.

The invention also provides host cells comprising DNA molecules according to the invention.

The invention further provides methods for generating host cells expressing a polypeptide of interest, the method comprising the steps of: introducing into a plurality of precursor host cells an expression cassette according to the invention, culturing the cells under conditions selecting for expression of the selectable marker polypeptide, and selecting at least one host cell producing the polypeptide of interest.

In a further aspect, the invention provides methods for producing a polypeptide of interest, the methods comprising culturing a host cell, said host cell comprising an expression cassette according to the invention, and expressing the polypeptide of interest from the expression cassette. In preferred embodiments thereof, the polypeptide of interest is further isolated from the host cells and/or from the host cell culture medium.

In further aspects, the invention provides RNA molecules having the sequence of a transcription product of a DNA molecule according to the invention.

In another aspect, the invention provides functional selectable marker polypeptides comprising a mutation as compared to their wild type sequence of their first amino acid from Methionine into either one of Valine (encoded by a GTG startcodon) or Leucine (encoded by a TTG startcodon), which polypeptides are obtainable by expression from certain DNA molecules according to the invention.

### BRIEF DESCRIPTION OF THE SEVERAL VIEWS OF THE DRAWINGS

FIG. 1. Schematic representation of the use of a selection marker gene (zeocin resistance gene) according to the invention of the incorporated '525 application. A. wild-type zeocin resitance gene, having its normal translation initation site (ATG startcodon) and one internal ATG codon, which codes for methionine. B. mutant zeocin resistance gene, wherein the internal ATG has been mutated into a codon for leucine; this mutant is a functional zeocin resistance gene. C. same as B, but comprising a mutated translation initiation site, wherein the context of the ATG startcodon has been mutated to decrease the translation initiation. D. same as B, but comprising a mutated startcodon (GTG). E. same as B, but with a TTG startcodon. The numbers under the figures C-E schematically indicate a relative amount of initiation frequency (under the startcodon) and ‘scan-through’ frequency (under the coding sequence) by the ribosomes, but only in a semi-quantitative manner, i.e. they indicate the efficiency of translation initiation compared to each other, but the qualitative numbers may differ completely: the numbers only serve to explain the invention. See example 1 for details.

FIG. 2. Schematic representation of a multicistronic transcription unit according to the invention of the incorporated '525 application, with more or less reciprocal interdependent translation efficiency. Explanation as for FIG. 1, but now a dEGFP gene (here exemplifying a gene of interest) has been placed downstream of the selectable marker polypeptide coding sequence. The Zeocin resistance gene comprises the internal Met→Leu mutation (see FIG. 1B). See example 2 for details.

FIG. 3. Results of selection systems according to the invention of the incorporated '525 application, with and without STAR elements. A. zeocin resistance gene with ATG startcodon in bad context (referred to as “ATGmut” in the picture, but including a spacer sequence behind the ATG in the bad context, so in the text generally referred to as “ATGmut/space”). B. zeocin resistance gene with GTG startcodon. C. zeocin resistance gene with TTG startcodon. d2EGFP signal for independent colonies is shown on the vertical axis. See example 2 for details.

FIG. 4. Results of selection system according to the invention of the incorporated '525 application in upscaled experiment (A), and comparison with selection system according to prior art using an IRES (B). d2EGFP signal for independent colonies is shown on the vertical axis. See example 3 for details.

FIG. 5. Results of selection system with multicistronic transcription unit according to the invention of the incorporated '525 application, using blasticidin as a selectable marker. A. blasticidin resistance gene mutated to comprise a GTG startcodon. B. blasticidin resistance gene mutated to comprise a TTG startcodon. The blasticidin resistance gene has further been mutated to remove all internal ATG sequences. d2EGFP signal for independent colonies is shown on the vertical axis. See example 4 for details.

FIG. 6. Stability of expression of several clones with a multicistronic transcription unit according to the invention (including a zeocin with TTG startcodon) of the incorporated '525 application. Selection pressure (100 μg/ml zeocin) was present during the complete experiment. d2EGFP signal for independent colonies is shown on the vertical axis. See example 5 for details.

FIG. 7. As FIG. 6, but zeocin concentration was lowered to 20 μg/ml after establishment of clones.

FIG. 8. As FIG. 6, but zeocin was absent from culture medium after establishment of clones.

FIG. 9. Expression of an antibody (anti-EpCAM) using the selection system with the multicistronic transcription unit according to the invention of the incorporated '525 application. The heavy chain (HC) and light chain (LC) are the polypeptide of interest in this example. Each of these is present in a separate transcription unit, which are both on a single nucleic acid molecule in this example. The HC is preceded by the zeocin resistance gene coding for a selectable marker polypeptide, while the LC is preceded by the blasticidin resistance gene coding for a selectable marker polypeptide. Both resistance genes have been mutated to comprise an ATG startcodon in a non-optimal context (“mutATG” in Figure, but including a spacer sequence, and hence in the text generally referred to as “ATGmut/space”). Each of the multicistronic transcription units is under control of a CMV promoter. Constructs with STAR sequences as indicated were compared to constructs without STAR sequences. The antibody levels obtained when these constructs were introduced into host cells are given on the vertical axis in pg/cell/day for various independent clones. See example 6 for details.

FIG. 10. As FIG. 9, but both the selection marker genes have been provided with a GTG startcodon. See example 6 for details.

FIG. 11. As FIG. 9, but both the selection marker genes have been provided with a TTG startcodon. See example 6 for details.

FIG. 12. Stability of expression in sub-clones in the absence of selection pressure (after establishing colonies under selection pressure, some colonies where sub-cloned in medium containing no zeocin). See example 5 for details.

FIG. 13. Copy-number dependency of expression levels of an embodiment of the invention of the incorporated '525 application. See example 5 for details.

FIG. 14. As FIG. 1, but for the blasticidin resistance gene. None of the 4 internal ATG's in this gene are in frame coding for a methionine, and therefore the redundancy of the genetic code was used to mutate these ATG's without mutating the internal amino acid sequence of the encoded protein.

FIG. 15. Coding sequence of the wild-type zeocin resistance gene (SEQ. ID. NO. 92). Bold ATG's code for methione. The first bold ATG is the startcodon.

FIG. 16. Coding sequence of the wild-type blasticidin resistance gene (SEQ. ID. NO. 94). Bold ATG's code for methione. The first bold ATG is the startcodon. Other ATG's in the sequence are underlined: these internal ATG's do not code for methionine, because they are not in frame.

FIG. 17. Coding sequence of the wild-type puromycin resistance gene (SEQ. ID. NO. 96). Bold ATG's code for methione. The first bold ATG is the startcodon.

FIG. 18. Coding sequence of the wild-type mouse DHFR gene (SEQ. ID. NO. 98). Bold ATG's code for methione. The first bold ATG is the startcodon. Other ATG's in the sequence are underlined: these internal ATG's do not code for methionine, because they are not in frame.

FIG. 19. Coding sequence of the wild-type hygromycin resistance gene (SEQ. ID. NO. 100). Bold ATG's code for methione. The first bold ATG is the startcodon. Other ATG's in the sequence are underlined: these internal ATG's do not code for methionine, because they are not in frame.

FIG. 20. Coding sequence of the wild-type neomycin resistance gene (SEQ. ID. NO. 102). Bold ATG's code for methione. The first bold ATG is the startcodon. Other ATG's in the sequence are underlined: these internal ATG's do not code for methionine, because they are not in frame.

FIG. 21. Coding sequence of the wild-type human glutamine synthase (GS) gene (SEQ. ID. NO. 104). Bold ATG's code for methione. The first bold ATG is the startcodon. Other ATG's in the sequence are underlined: these internal ATG's do not code for methionine, because they are not in frame.

FIG. 22. Schematic representation of some further modified zeocin resistance selection marker genes with a GTG startcodon according to the invention, allowing for further fine-tuning of the selection stringency. See example 7 for details.

FIG. 23. Results with expression systems containing the further modified zeocin resistance selection marker genes. See example 7 for details. Dots indicate individual data points; lines indicate the average expression levels; used constructs (see also FIG. 22) are indicated on the horizontal axis (the addition of 7/67/7 at the end of the construct name indicates the presence of STAR sequences 7 and 67 upstream of the promoter and STAR7 downstream of the transcription termination site), and schematically depicted above the graph; vertical axis indicates d2EGFP signal.

FIG. 24. Schematic representation of some further modified zeocin resistance selection marker genes with a TTG startcodon according to the invention, allowing for further fine-tuning of the selection stringency. See example 8 for details.

FIG. 25. Results with expression systems containing the further modified zeocin resistance selection marker genes. See example 8 for details. Dots indicate individual data points; lines indicate the average expression levels; used constructs are indicated on the horizontal axis, and schematically depicted above the graph; vertical axis indicates d2EGFP signal.

FIG. 26. As FIG. 1, but for the puromycin resistance gene. All three internal ATG's code for methione (panel A), and are replaced by CTG sequences coding for leucine (panel B). See example 9 for details.

FIG. 27. Results with expression constructs containing the puromycin resistance gene with a TTG startcodon and no internal ATG codons. See example 9 for details. Dots indicate individual data points; lines indicate the average expression levels; used constructs are indicated on the horizontal axis, and schematically depicted above the graph; vertical axis indicates d2EGFP signal.

FIG. 28. As FIG. 1, but for the neomycin resistance gene. See Example 10 for details. A. wild-type neomycin resistance gene; ATG sequences are indicated, ATGs coding for methionine are indicated by Met above the ATG. B. neomycin resistance gene without ATG sequences, and with a GTG startcodon. C. neomycin resistance gene without ATG sequences, and with a TTG startcodon.

FIG. 29. As FIG. 1, but for the dhfr gene. See Example 11 for details. A. wild-type dhfr gene; ATG sequences are indicated, ATGs coding for methionine are indicated by Met above the ATG. B. dhfr gene without ATG sequences, and with a GTG startcodon. C. dhfr gene without ATG sequences, and with a TTG startcodon.

FIG. 30. Results with expression constructs (zeocin selectable marker) according to the invention of the incorporated '525 application in PER.C6 cells. See Example 12 for details. Dots indicate individual data points; lines indicate the average expression levels; used constructs are indicated on the horizontal axis, and schematically depicted above the graph; vertical axis indicates d2EGFP signal.

FIG. 31. Results with expression constructs (blasticidin selectable marker) according to the invention of the incorporated '525 application in PER.C6 cells. See Example 12 for details. Dots indicate individual data points; lines indicate the average expression levels; used constructs are indicated on the horizontal axis, and schematically depicted above the graph; vertical axis indicates d2EGFP signal.

FIG. 32. Results with expression constructs according to the invention of the incorporated '525 application, further comprising a transcription pause (TRAP) sequence. See Example 13 for details. Dots indicate individual data points; lines indicate the average expression levels; used constructs are indicated on the horizontal axis, and schematically depicted above the graph; vertical axis indicates d2EGFP signal.

FIG. 33. Copy-number dependency of expression of an antibody using transcription units according to the invention of the incorporated '525 application. See Example 14 for details.

FIG. 34. Antibody expression from colonies containing expression constructs according to the invention of the incorporated '525 application, wherein the copy number of the expression constructs is amplified by methotrexate. See Example 15 for details. White bars: selection with zeocin and blasticidin; black bars: selection with zeocin, blasticidin and methotrexate (MTX). Numbers of tested colonies are depicted on the horizontal axis.

FIG. 35. Results with different promoters. See Example 16 for details. Dots indicate individual data points; lines indicate the average expression levels; used constructs are indicated on the horizontal axis, and schematically depicted above the graph; vertical axis indicates d2EGFP signal.

FIG. 36. Results with different STAR elements. See example 17 for details. Dots indicate individual data points; lines indicate the average expression levels; used constructs are indicated on the horizontal axis, and schematically depicted above the graph; vertical axis indicates d2EGFP signal.

FIG. 37. Results with other chromatin control elements. See Example 18 for details. Dots indicate individual data points; lines indicate the average expression levels; used constructs are indicated on the horizontal axis, and schematically depicted above the graph (black triangles indicate different tested chromatin control elements); vertical axis indicates d2EGFP signal.

FIG. 38. Results with expression constructs according to the invention. The expression construct contains the sequence encoding the polypeptide of interest (exemplified here by d2EGFP) upstream of an IRES, which is upstream of the sequence encoding the selectable marker according to the invention (exemplified here by the zeocin resistance gene, with a TTG startcodon (TTG Zeo) (or in controls with its normal ATG startcodon (ATG Zeo)). See example 19 for details. Dots indicate individual data points; lines indicate the average expression levels; used constructs are indicated on the horizontal axis, and schematically depicted above the graph; vertical axis indicates d2EGFP signal.

### DETAILED DESCRIPTION OF THE INVENTION

In one aspect, the invention provides a DNA molecule comprising a multicistronic transcription unit coding for i) a polypeptide of interest, and for ii) a selectable marker polypeptide functional in a eukaryotic host cell, wherein the polypeptide of interest has a translation initiation sequence separate from that of the selectable marker polypeptide, and wherein the coding sequence for the polypeptide of interest is upstream from the coding sequence for the selectable marker polypeptide in said multicistronic transcription unit, and wherein an internal ribosome entry site (IRES) is present downstream from the coding sequence for the polypeptide of interest and upstream from the coding sequence for the selectable marker polypeptide, and wherein the nucleic acid sequence coding for the selectable marker polypeptide in the coding strand comprises a translation start sequence chosen from the group consisting of: a) an ATG startcodon in a non-optimal context for translation initiation, comprising the sequence (C/T)(A/T/G)(A/T/G)ATG(A/T/C) wherein the startcodon is underlined; b) a GTG startcodon; c) a TTG startcodon; d) a CTG startcodon; e) a ATT startcodon; and f) a ACG startcodon. Such a DNA molecule can be used according to the invention for obtaining eukaryotic host cells expressing high levels of the polypeptide of interest, by selecting for the expression of the selectable marker polypeptide. Subsequently or simultaneously, one or more host cell(s) expressing the polypeptide of interest can be identified, and further used for expression of high levels of the polypeptide of interest.

The term “monocistronic gene” is defined as a gene capable of providing a RNA molecule that encodes one polypeptide. A “multicistronic transcription unit”, also referred to as multicistronic gene, is defined as a gene capable of providing an RNA molecule that encodes at least two polypeptides. The term “bicistronic gene” is defined as a gene capable of providing a RNA molecule that encodes two polypeptides. A bicistronic gene is therefore encompassed within the definition of a multicistronic gene. A “polypeptide” as used herein comprises at least five amino acids linked by peptide bonds, and can for instance be a protein or a part, such as a subunit, thereof. Mostly, the terms polypeptide and protein are used interchangeably herein. A “gene” or a “transcription unit” as used in the present invention can comprise chromosomal DNA, cDNA, artificial DNA, combinations thereof, and the like. Transcription units comprising several cistrons are transcribed as a single mRNA.

A multicistronic transcription unit according to the invention preferably is a bicistronic transcription unit coding from 5′ to 3′ for a polypeptide of interest and for a selectable marker polypeptide. Hence, the polypeptide of interest is encoded upstream from the coding sequence for the selectable marker polypeptide. The IRES is operably linked to the sequence encoding the selectable marker polypeptide, and hence the selectable marker polypeptide is dependent from the IRES for its translation.

It is preferred to use separate transcription units for the expression of different polypeptides of interest, also when these form part of a multimeric protein (see e.g. example 6: the heavy and light chain of an antibody each are encoded by a separate transcription unit, each of these expression units being a bicistronic expression unit).

The DNA molecules of the invention can be present in the form of double stranded DNA, having with respect to the selectable marker polypeptide and the polypeptide of interest a coding strand and a non-coding strand, the coding strand being the strand with the same sequence as the translated RNA, except for the presence of T instead of U. Hence, an AUG startcodon is coded for in the coding strand by an ATG sequence, and the strand containing this ATG sequence corresponding to the AUG startcodon in the RNA is referred to as the coding strand of the DNA. It will be clear to the skilled person that startcodons or translation initiation sequences are in fact present in an RNA molecule, but that these can be considered equally embodied in a DNA molecule coding for such an RNA molecule; hence, wherever the present invention refers to a startcodon or translation initation sequence, the corresponding DNA molecule having the same sequence as the RNA sequence but for the presence of a T instead of a U in the coding strand of said DNA molecule is meant to be included, and vice versa, except where explicitly specified otherwise. In other words, a startcodon is for instance an AUG sequence in RNA, but the corresponding ATG sequence in the coding strand of the DNA is referred to as startcodon as well in the present invention. The same is used for the reference of ‘in frame’ coding sequences, meaning triplets (3 bases) in the RNA molecule that are translated into an amino acid, but also to be interpreted as the corresponding trinucleotide sequences in the coding strand of the DNA molecule.

The selectable marker polypeptide and the polypeptide of interest encoded by the multicistronic gene each have their own translation initation sequence, and therefore each have their own startcodon (as well as stopcodon), i.e. they are encoded by separate open reading frames.

The term “selection marker” or “selectable marker” is typically used to refer to a gene and/or protein whose presence can be detected directly or indirectly in a cell, for example a polypeptide that inactivates a selection agent and protects the host cell from the agent's lethal or growth-inhibitory effects (e.g. an antibiotic resistance gene and/or protein). Another possibility is that said selection marker induces fluorescence or a color deposit (e.g. green fluorescent protein (GFP) and derivatives (e.g d2EGFP), luciferase, lacZ, alkaline phosphatase, etc.), which can be used for selecting cells expressing the polypeptide inducing the color deposit, e.g. using a fluorescence activated cell sorter (FACS) for selecting cells that express GFP. Preferably, the selectable marker polypeptide according to the invention provides resistance against lethal and/or growth-inhibitory effects of a selection agent. The selectable marker polypeptide is encoded by the DNA of the invention. The selectable marker polypeptide according to the invention must be functional in a eukaryotic host cell, and hence being capable of being selected for in eukaryotic host cells. Any selectable marker polypeptide fulfilling this criterion can in principle be used according to the present invention. Such selectable marker polypeptides are well known in the art and routinely used when eukaryotic host cell clones are to be obtained, and several examples are provided herein. In certain embodiments, a selection marker used for the invention is zeocin. In other embodiments, blasticidin is used. The person skilled in the art will know that other selection markers are available and can be used, e.g. neomycin, puromycin, bleomycin, hygromycin, etc. In other embodiments, kanamycin is used. In yet other embodiments, the DHFR gene is used as a selectable marker, which can be selected for by methotrexate, especially by increasing the concentration of methotrexate cells can be selected for increased copy numbers of the DHFR gene. Similarly, the glutamine synthetase (GS) gene can be used, for which selection is possible in cells having insufficient GS (e.g. NS-0 cells) by culturing in media without glutamine, or alternatively in cells having sufficient GS (e.g. CHO cells) by adding an inhibitor of GS, methionine sulphoximine (MSX). Other selectable marker genes that could be used, and their selection agents, are for instance described in table 1 of U.S. Pat. No. 5,561,053, incorporated by reference herein; see also Kaufman, Methods in Enzymology, 185:537-566 (1990), for a review of these.

When two multicistronic transcription units are to be selected for according to the invention in a single host cell, each one preferably contains the coding sequence for a different selectable marker, to allow selection for both multicistronic transcription units. Of course, both multicistronic transcription units may be present on a single nucleic acid molecule or alternatively each one may be present on a separate nucleic acid molecule.

The term “selection” is typically defined as the process of using a selection marker/selectable marker and a selection agent to identify host cells with specific genetic properties (e.g. that the host cell contains a transgene integrated into its genome). It is clear to a person skilled in the art that numerous combinations of selection markers are possible. One antibiotic that is particularly advantageous is zeocin, because the zeocin-resistance protein (zeocin-R) acts by binding the drug and rendering it harmless. Therefore it is easy to titrate the amount of drug that kills cells with low levels of zeocin-R expression, while allowing the high-expressors to survive. All other antibiotic-resistance proteins in common use are enzymes, and thus act catalytically (not 1:1 with the drug). Hence, the antibiotic zeocin is a preferred selection marker. However, the invention also works with other selection markers.

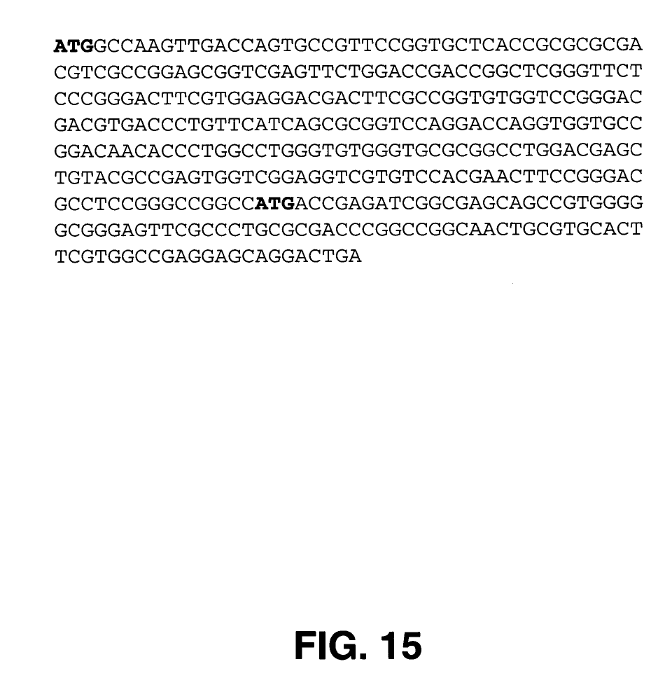

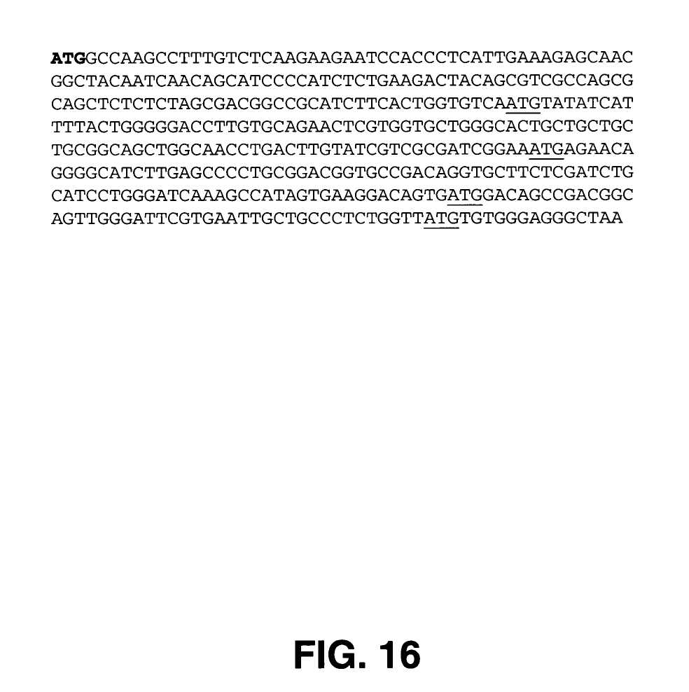

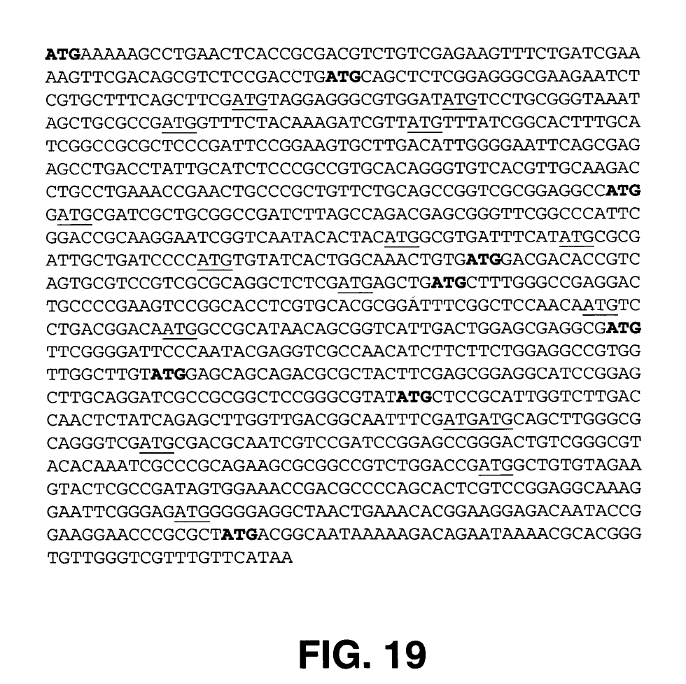

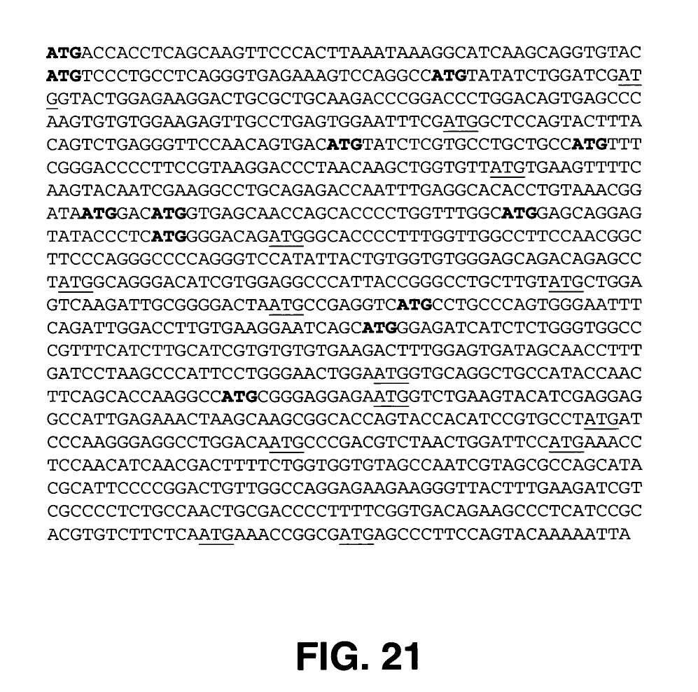

A selectable marker polypeptide according to the invention is the protein that is encoded by the nucleic acid of the invention, which polypeptide can be detected, for instance because it provides resistance to a selection agent such as an antibiotic. Hence, when an antibiotic is used as a selection agent, the DNA encodes a polypeptide that confers resistance to the selection agent, which polypeptide is the selectable marker polypeptide. DNA sequences coding for such selectable marker polypeptides are known, and several examples of wild-type sequences of DNA encoding selectable marker proteins are provided herein (FIGS. 15-21). It will be clear that mutants or derivatives of selectable markers can also be suitably used according to the invention, and are therefore included within the scope of the term ‘selectable marker polypeptide’, as long as the selectable marker protein is still functional.

For convenience and as generally accepted by the skilled person, in many publications as well as herein, often the gene and protein encoding the resistance to a selection agent is referred to as the ‘selectable agent (resistance) gene’ or ‘selection agent (resistance) protein’, respectively, although the official names may be different, e.g. the gene coding for the protein conferring restance to neomycin (as well as to G418 and kanamycin) is often referred to as neomycin (resistance) (or neo^{r}) gene, while the official name is aminoglycoside 3′-phosphotransferase gene.

For the present invention, it is beneficial to have low levels of expression of the selectable marker polypeptide, so that stringent selection is possible. In the present invention this is brought about by using a selectable marker coding sequence with a non-optimal translation efficiency. Upon selection, only cells that have nevertheless sufficient levels of selectable marker polypeptide will be selected, meaning that such cells must have sufficient transcription of the multicistronic transcription unit and sufficient translation of the selectable marker polypeptide, which provides a selection for cells where the multicistronic transcription unit has been integrated or otherwise present in the host cells at a place where expression levels from this transcription unit are high.

The DNA molecules according to the invention have the coding sequence for the selectable marker polypeptide downstream of the coding sequence for the polypeptide of interest. Hence, the multicistronic transcription unit comprises in the 5′ to 3′ direction (both in the transcribed strand of the DNA and in the resulting transcribed RNA) the sequence encoding the polypeptide of interest and the coding sequence for the selectable marker polypeptide. The IRES is upstream of the coding sequence for the selectable marker polypeptide.

According to the invention, the coding region of the gene of interest is preferably translated from the cap-dependent ORF, and the polypeptide of interest is produced in abundance. The selectable marker polypeptide is translated from an IRES. To decrease translation of the selectable marker cistron, according to the invention the nucleic acid sequence coding for the selectable marker polypeptide comprises a mutation in the startcodon (or in the context thereof) that decreases the translation initiation efficiency of the selectable marker polypeptide in a eukaryotic host cell. Preferably, a GTG startcodon or more prefereably a TTG startcodon is engineered into the selectable marker polypeptide. The translation efficiency is lower than that of the corresponding wild-type sequence in the same cell, i.e. the mutation results in less polypeptide per cell per time unit, and hence less selectable marker polypeptide. This can be detected using routine methods known to the person skilled in the art. For instance in the case of antibiotic selection the mutation will result in less resistance than obtained with the sequence having no such mutation and hence normal translation efficiency, which difference can easily be detected by determining the number of surviving colonies after a normal selection period, which will be lower when a translation efficiency decreasing mutation is present. As is well known to the person skilled in the art there are a number of parameters that indicate the expression level marker polypeptide such as, the maximum concentration of selection agent to which cells are still resistant, number of surviving colonies at a given concentration, growth speed (doubling time) of the cells in the presence of selection agent, combinations of the above, and the like.

The mutation that decreases the translation initiation efficiency according to the invention is established by providing the selectable marker polypeptide coding sequence with a non-optimal translation start sequence.

For example, the translation initiation efficiency of the selectable marker gene in eukaryotic cells can be suitably decreased according to the invention by mutating the startcodon and/or the nucleotides in positions −3 to −1 and +4 (where the A of the ATG startcodon is nt +1), for instance in the coding strand of the corresponding DNA sequence, to provide a non-optimal translation start sequence. A translation start sequence is often referred to in the field as ‘Kozak sequence’, and an optimal Kozak sequence is RCCATGG, the startcodon underlined, R being a purine, i.e. A or G (see Kozak M, 1986, 1987, 1989, 1990, 1997, 2002). Hence, besides the startcodon itself, the context thereof, in particular nucleotides −3 to −1 and +4, are relevant, and an optimal translation startsequence comprises an optimal startcodon (i.e. ATG) in an optimal context (i.e. the ATG directly preceded by RCC and directly followed by G). A non-optimal translation start sequence is defined herein as any sequence that gives at least some detectable translation in a eukaryotic cell (detectable because the selection marker polypeptide is detectable), and not having the consensus sequence RCCATGG (startcodon underlined). Translation by the ribosomes is most efficient when an optimal Kozak sequence is present (see Kozak M, 1986, 1987, 1989, 1990, 1997, 2002). However, in a small percentage of events, non-optimal translation initiation sequences are recognized and used by the ribosome to start translation. The present invention makes use of this principle, and allows for decreasing and even fine-tuning of the amount of translation and hence expression of the selectable marker polypeptide, which can therefore be used to increase the stringency of the selection system.

In a first embodiment of the invention, the ATG startcodon of the selectable marker polypeptide (in the coding strand of the DNA, coding for the corresponding AUG startcodon in the RNA transcription product) is left intact, but the positions at −3 to −1 and +4 are mutated such that they do not fulfill the optimal Kozak sequence any more, e.g. by providing the sequence TTTATGT as the translation start site (ATG startcodon underlined). It will be clear that other mutations around the startcodon at positions −3 to −1 and/or +4 could be used with similar results using the teaching of the present invention, as can be routinely and easily tested by the person skilled in the art. The idea of this first embodiment is that the ATG startcodon is placed in a ‘non-optimal’ context for translation initiation.

In a second and preferred embodiment, the ATG startcodon itself of the selectable marker polypeptide is mutated. This will in general lead to even lower levels of translation initiation than the first embodiment. The ATG startcodon in the second embodiment is mutated into another codon, which has been reported to provide some translation initiation, for instance to GTG, TTG, CTG, ATT, or ACG (collectively referred to herein as ‘non-optimal start codons’). In preferred embodiments, the ATG startcodon is mutated into a GTG startcodon. This provides still lower expression levels (lower translation) than with the ATG startcodon intact but in a non-optimal context. More preferably, the ATG startcodon is mutated to a TTG startcodon, which provides even lower expression levels of the selectable marker polypeptide than with the GTG startcodon (Kozak M, 1986, 1987, 1989, 1990, 1997, 2002; see also examples 2-6 herein). The use of non-ATG startcodons in the coding sequence for a selectable marker polypeptide in a multicistronic transcription unit according to the present invention was not disclosed nor suggested in the prior art and, preferably in combination with chromatin control elements, leads to very high levels of expression of the polypeptide of interest, as also shown in the incorporated '525 application.

For the second embodiment, i.e. where a non-ATG startcodon is used, it is strongly preferred to provide an optimal context for such a startcodon, i.e. the non-optimal startcodons are preferably directly preceded by nucleotides RCC in positions −3 to −1 and directly followed by a G nucleotide (position +4). However, it has been reported that using the sequence TTTGTGG (startcodon underlined), some initiation is observed at least in vitro, so although strongly preferred it may not be absolutely required to provide an optimal context for the non-optimal startcodons.

ATG sequences within the coding sequence for a polypeptide, but excluding the ATG startcodon, are referred to as ‘internal ATGs’, and if these are in frame with the ORF and therefore code for methionine, the resulting methionine in the polypeptide is referred to as an ‘internal methionine’. It is strongly preferred according to the invention of the incorporated '525 application that the coding region (following the startcodon, not necessarily including the startcodon) coding for the selectable marker polypeptide is devoid of any ATG sequence in the coding strand of the DNA, up to (but not including) the startcodon of the polypeptide of interest (obviously, the startcodon of the polypeptide of interest may be, and in fact preferably is, an ATG startcodon). The incorporated '525 application discloses how to bring this about and how to test the resulting selectable marker polypeptides for functionality. For the present invention, where the selectable marker polypeptide coding sequence is downstream of an IRES and downstream of the coding sequence for the polypeptide of interest, internal ATGs in the sequence encoding the selectable marker polypeptide can remain intact.

Clearly, it is strongly preferred according to the present invention, that the translation start sequence of the polypeptide of interest comprises an optimal translation start sequence, i.e. having the consensus sequence RCCATGG (startcodon underlined). This will result in a very efficient translation of the polypeptide of interest.

By providing the coding sequence of the marker with different mutations leading to several levels of decreased translation efficiency, the stringency of selection can be increased. Fine-tuning of the selection system is thus possible using the multicistronic transcription units according to the invention: for instance using a GTG startcodon for the selection marker polypeptide, only few ribosomes will translate from this startcodon, resulting in low levels of selectable marker protein, and hence a high stringency of selection; using a TTG startcodon even further increases the stringency of selection because even less ribosomes will translate the selectable marker polypeptide from this startcodon.

It is demonstrated in the incorporated '525 application that the multicistronic expression units disclosed therein can be used in a very robust selection system, leading to a very large percentage of clones that express the polypeptide of interest at high levels, as desired. In addition, the expression levels obtained for the polypeptide of interest appear to be significantly higher than those obtained when an even larger number of colonies are screened using selection systems hitherto known.

In addition to a decreased translation initiation efficiency, it could be beneficial to also provide for decreased translation elongation efficiency of the selectable marker polypeptide, e.g. by mutating the coding sequence thereof so that it comprises several non-preferred codons of the host cell, in order to further decrease the translation levels of the marker polypeptide and allow still more stringent selection conditions, if desired. In certain embodiments, besides the mutation(s) that decrease the translation efficiency according to the invention, the selectable marker polypeptide further comprises a mutation that reduces the activity of the selectable marker polypeptide compared to its wild-type counterpart. This may be used to increase the stringency of selection even further. As non-limiting examples, proline at position 9 in the zeocin resistance polypeptide may be mutated, e.g. to Thr or Phe, and for the neomycin resistance polypeptide, amino acid residue 182 or 261 or both may further be mutated (see e.g. WO 01/32901).

In some embodiments of the invention, a so-called spacer sequence is placed downstream of the sequence encoding the startcodon of the selectable marker polypeptide, which spacer sequence preferably is a sequence in frame with the startcodon and encoding a few amino acids, and that does not contain a secondary structure (Kozak, 1990), and does not contain the sequence ATG. Such a spacer sequence can be used to further decrease the translation initiation frequency if a secondary structure is present in the RNA (Kozak, 1990) of the selectable marker polypeptide (e.g. for zeocin, possibly for blasticidin), and hence increase the stringency of the selection system according to the invention.

The invention also provides a DNA molecule comprising the sequence encoding a selectable marker protein according to the invention, which DNA molecule has been provided with a mutation that decreases the translation efficiency of the functional selectable marker polypeptide in a eukarytic host cell. In preferred embodiments hereof, said DNA molecule in the coding strand has been mutated compared to the wild-type sequence encoding said selectable marker polypeptide, such that the sequence ATG of the startcodon is mutated into GTG (encoding Valine) or into TTG (encoding Leucine), and wherein the selectable marker polypeptide is still functional in a eukaryotic host cell. Such DNA molecules encompass a useful intermediate product according to the invention. These molecules can be prepared first, introduced into eukaryotic host cells and tested for functionality (for some markers this is even possible in prokaryotic host cells), if desired in a (semi-) quantitative manner, of the selectable marker polypeptide. They may then be further used to prepare a DNA molecule according to the invention, comprising the multicistronic transcription unit.

In one embodiment thereof, the invention provides a DNA molecule comprising a DNA sequence encoding a protein that confers resistance to zeocin, said DNA sequence comprising SEQ. ID. NO. 92, with the proviso that the first ATG (the startcodon, encoding Methionine) is replaced by either a GTG (encoding Valine) or a TTG (encoding Leucine) startcodon.

In another embodiment thereof, the invention provides a DNA molecule comprising a DNA sequence encoding a protein that confers resistance to blasticidin, said DNA sequence comprising SEQ. ID. NO. 94, with the proviso that the first ATG (the startcodon, encoding Methionine) is replaced by either a GTG (encoding Valine) or a TTG (encoding Leucine) startcodon.

In another embodiment thereof, the invention provides a DNA molecule comprising a DNA sequence encoding a protein that confers resistance to neomycin, said DNA sequence comprising SEQ. ID. NO. 102, with the proviso that the first ATG (the startcodon, encoding Methionine) is replaced by either a GTG (encoding Valine) or a TTG (encoding Leucine) startcodon.

In another embodiment thereof, the invention provides a DNA molecule comprising a DNA sequence encoding a protein that confers resistance to puromycin, said DNA sequence comprising SEQ. ID. NO. 96, with the proviso that the first ATG (the startcodon, encoding Methionine) is replaced by either a GTG (encoding Valine) or a TTG (encoding Leucine) startcodon.

In another embodiment thereof, the invention provides a DNA molecule comprising a DNA sequence encoding a protein that confers resistance to hygromycin, said DNA sequence comprising SEQ. ID. NO. 100, with the proviso that the first ATG (the startcodon, encoding Methionine) is replaced by either a GTG (encoding Valine) or a TTG (encoding Leucine) startcodon.

In another embodiment thereof, the invention provides a DNA molecule comprising a DNA sequence encoding a protein with dihydrofolate reductase (dhfr) activity (conferring resistance to methotrexate), said DNA sequence comprising SEQ. ID. NO. 98, with the proviso that the first ATG (the startcodon, encoding Methionine) is replaced by either a GTG (encoding Valine) or a TTG (encoding Leucine) startcodon.

In another embodiment thereof, the invention provides a DNA molecule comprising a DNA sequence encoding a protein with glutamine synthetase (GS) activity, said DNA sequence comprising SEQ. ID. NO. 104, with the proviso that the first ATG (the startcodon, encoding Methionine) is replaced by either a GTG (encoding Valine) or a TTG (encoding Leucine) startcodon.

It will be clear that for these embodiments, any DNA molecules as described but having mutations in the sequence downstream of the first ATG (startcodon) coding for the selectable marker protein are also encompassed in the invention, as long as the respective encoded selectable marker protein still has activity. For instance any silent mutations that do not alter the encoded protein because of the redundancy of the genetic code are also encompassed. Further mutations that lead to conservative amino acid mutations or to other mutations are also encompassed, as long as the encoded protein still has activity, which may or may not be lower than that of the wild-type protein as encoded by the indicated sequences. In particular, it is preferred that the encoded protein is at least 70%, preferably at least 80%, more preferably at least 90%, still more preferably at least 95% identical to the proteins encoded by the respective indicated sequences. Testing for activity of the selectable marker proteins can be done by routine methods.

The invention also provides the selectable marker proteins encoded by these embodiments.

It is a preferred aspect of the invention to provide an expression cassette comprising the DNA molecule according to the invention, having the multicistronic transcription unit. Such an expression cassette is useful to express sequences of interest, for instance in host cells. An ‘expression cassette’ as used herein is a nucleic acid sequence comprising at least a promoter functionally linked to a sequence of which expression is desired. Preferably, an expression cassette further contains transcription termination and polyadenylation sequences. Other regulatory sequences such as enhancers may also be included. Hence, the invention provides an expression cassette comprising in the following order: 5′—promoter—multicistronic transcription unit according to the invention, coding for a polypeptide of interest and downstream thereof a selectable marker polypeptide—transcription termination sequence—3′. The promoter must be capable of functioning in a eukaryotic host cell, i.e. it must be capable of driving transcription of the multicistronic transcription unit. The promoter is thus operably linked to the multicistronic transcription unit. The expression cassette may optionally further contain other elements known in the art, e.g. splice sites to comprise introns, and the like. In some embodiments, an intron is present behind the promoter and before the sequence encoding the polypeptide of interest. An IRES is operably linked to the cistron that contains the selectable marker polypeptide coding sequence.

To obtain expression of nucleic acid sequences encoding protein, it is well known to those skilled in the art that sequences capable of driving such expression, can be functionally linked to the nucleic acid sequences encoding the protein, resulting in recombinant nucleic acid molecules encoding a protein in expressible format. In the present invention, the expression cassette comprises a multicistronic transcription unit. In general, the promoter sequence is placed upstream of the sequences that should be expressed. Much used expression vectors are available in the art, e.g. the pcDNA and pEF vector series of Invitrogen, pMSCV and pTK-Hyg from BD Sciences, pCMV-Script from Stratagene, etc, which can be used to obtain suitable promoters and/or transcription terminator sequences, polyA sequences, and the like.

Where the sequence encoding the polypeptide of interest is properly inserted with reference to sequences governing the transcription and translation of the encoded polypeptide, the resulting expression cassette is useful to produce the polypeptide of interest, referred to as expression. Sequences driving expression may include promoters, enhancers and the like, and combinations thereof. These should be capable of functioning in the host cell, thereby driving expression of the nucleic acid sequences that are functionally linked to them. The person skilled in the art is aware that various promoters can be used to obtain expression of a gene in host cells. Promoters can be constitutive or regulated, and can be obtained from various sources, including viruses, prokaryotic, or eukaryotic sources, or artificially designed. Expression of nucleic acids of interest may be from the natural promoter or derivative thereof or from an entirely heterologous promoter (Kaufman, 2000). Some well-known and much used promoters for expression in eukaryotic cells comprise promoters derived from viruses, such as adenovirus, e.g. the E1A promoter, promoters derived from cytomegalovirus (CMV), such as the CMV immediate early (IE) promoter (referred to herein as the CMV promoter) (obtainable for instance from pcDNA, Invitrogen), promoters derived from Simian Virus 40 (SV40) (Das et al, 1985), and the like. Suitable promoters can also be derived from eukaryotic cells, such as methallothionein (MT) promoters, elongation factor 1α (EF-1α) promoter (Gill et al., 2001), ubiquitin C or UB6 promoter (Gill et al., 2001; Schorpp et al, 1996), actin promoter, an immunoglobulin promoter, heat shock promoters, and the like. Some preferred promoters for obtaining expression in eukaryotic cells, which are suitable promoters in the present invention, are the CMV-promoter, a mammalian EF1-alpha promoter, a mammalian ubiquitin promoter such as a ubiquitin C promoter, or a SV40 promoter (e.g. obtainable from pIRES, cat.no. 631605, BD Sciences). Testing for promoter function and strength of a promoter is a matter of routine for a person skilled in the art, and in general may for instance encompass cloning a test gene such as lacZ, luciferase, GFP, etc. behind the promoter sequence, and test for expression of the test gene. Of course, promoters may be altered by deletion, addition, mutation of sequences therein, and tested for functionality, to find new, attenuated, or improved promoter sequences. According to the present invention, strong promoters that give high transcription levels in the eukaryotic cells of choice are preferred.

In certain embodiments, a DNA molecule according to the invention is part of a vector, e.g. a plasmid. Such vectors can easily be manipulated by methods well known to the person skilled in the art, and can for instance be designed for being capable of replication in prokaryotic and/or eukaryotic cells. In addition, many vectors can directly or in the form of isolated desired fragment therefrom be used for transformation of eukaryotic cells and will integrate in whole or in part into the genome of such cells, resulting in stable host cells comprising the desired nucleic acid in their genome.

Conventional expression systems are DNA molecules in the form of a recombinant plasmid or a recombinant viral genome. The plasmid or the viral genome is introduced into (eukaryotic host) cells and preferably integrated into their genomes by methods known in the art. In preferred embodiments, the present invention also uses these types of DNA molecules to deliver its improved transgene expression system. A preferred embodiment of the invention is the use of plasmid DNA for delivery of the expression system. A plasmid contains a number of components: conventional components, known in the art, are an origin of replication and a selectable marker for propagation of the plasmid in bacterial cells; a selectable marker that functions in eukaryotic cells to identify and isolate host cells that carry an integrated transgene expression system; the protein of interest, whose high-level transcription is brought about by a promoter that is functional in eukaryotic cells (e.g. the human cytomegalovirus major immediate early promoter/enhancer, pCMV (Boshart et al., 1985); and viral transcriptional terminators (e.g. the SV40 polyadenylation site (Kaufman & Sharp, 1982) for the transgene of interest and the selectable marker.

The vector used can be any vector that is suitable for cloning DNA and that can be used for transcription of a nucleic acid of interest. When host cells are used it is preferred that the vector is an integrating vector. Alternatively, the vector may be an episomally replicating vector.

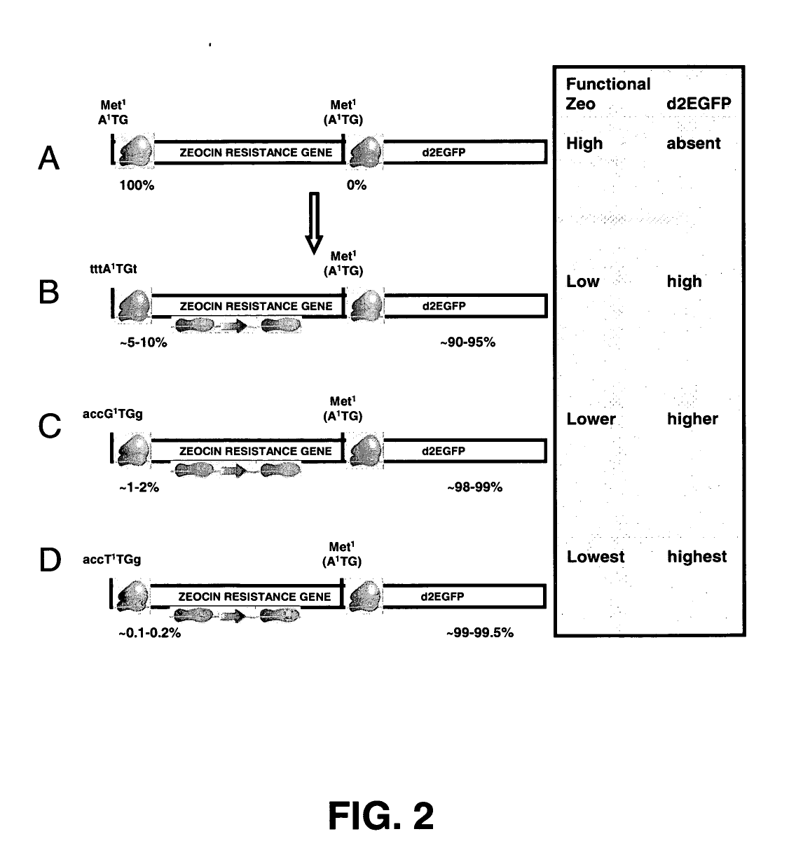

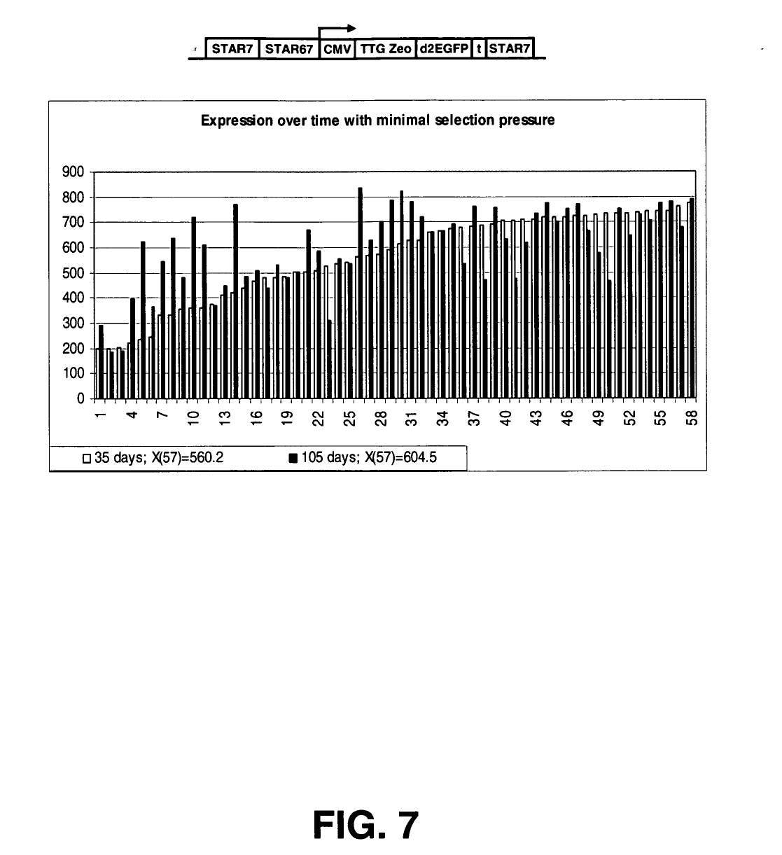

It is widely appreciated that chromatin structure and other epigenetic control mechanisms may influence the expression of transgenes in eukaryotic cells (e.g. Whitelaw et al, 2001). The multicistronic expression units according to the invention form part of a selection system with a rather rigourous selection regime. This generally requires high transcription levels in the host cells of choice. To increase the chance of finding clones of host cells that survive the rigorous selection regime, and possibly to increase the stability of expression in obtained clones, it will generally be preferable to increase the predictability of transcription. Therefore, in preferred embodiments, an expression cassette according to the invention further comprises at least one chromatin control element. A ‘chromatin control element’ as used herein is a collective term for DNA sequences that may somehow have an effect on the chromatin structure and therewith on the expression level and/or stability of expression of transgenes in their vicinity (they function ‘in cis’, and hence are placed preferably within 5 kb, more preferably within 2 kb, still more preferably within 1 kb from the transgene) within eukaryotic cells. Such elements have sometimes been used to increase the number of clones having desired levels of transgene expression. The mechanisms by which these elements work may differ for and even within different classes of such elements, and are not completely known for all types of such elements. However, such elements have been described, and for the purpose of the present invention chromatin control elements are chosen from the group consisting of matrix or scaffold attachment regions (MARs/SARs) (e.g. Phi-Van et al, 1990; WO 02/074969, WO 2005/040377), insulators (West et al, 2002) such as the beta-globin insulator element (5′ HS4 of the chicken beta-globin locus), scs, scs', and the like (e.g. Chung et al, 1993, 1997; Kellum and Schedl, 1991; WO 94/23046, WO 96/04390, WO 01/02553, WO 2004/027072), a ubiquitous chromatin opening element (UCOE) (WO 00/05393, WO 02/24930, WO 02/099089, WO 02/099070), and anti-repressor sequences (also referred to as ‘STAR’ sequences) (Kwaks et al, 2003; WO 03/004704). Non-limiting examples of MAR/SAR sequences that could be used in the current invention are the chicken lysosyme 5′ MAR (Phi-Van et al, 1990) or fragments thereof, e.g. the B, K and F regions as described in WO 02/074969); DNA sequences comprising at least one bent DNA element and at least one binding site for a DNA binding protein, preferably containing at least 10% of dinucleotide TA, and/or at least 12% of dinucleotide AT on a stretch of 100 contiguous base pairs, such as a sequence selected from the group of comprising the sequences SEQ ID Nos 1 to 27 in WO 2005/040377, fragments of any one of SEQ ID Nos 1 to 27 in WO 2005/040377 being at least 100 nucleotides in length and having MAR activity, sequences that are at least 70% identical in nucleotide sequence to any one of SEQ ID Nos 1 to 27 in WO 2005/040377 or fragments thereof and having MAR activity, wherein MAR activity is defined as being capable of binding to nuclear matrices/scaffolds in vitro and/or of altering the expression of coding sequences operably linked to a promoter; sequences chosen from any one of SEQ ID NO: 1 to 5 in WO 02/074969, fragments of any one of any one of SEQ ID NO: 1 to 5 in WO 02/074969 and having MAR activity, sequences that are at least 70% identical in nucleotide sequence to any one of SEQ ID NO: 1 to 5 in WO 02/074969 or fragments thereof and having MAR activity; sequences chosen from SEQ ID NO: 1 and SEQ ID NO: 2 in WO 2004/027072, functional fragments thereof and sequences being at least 70% identical thereto. A non-limiting example of insulator sequences that could be used in the present invention is a sequence that comprises SEQ ID NO:1 of WO 01/02553. Non-limiting examples of UCOEs that could be used in the present invention are sequences depicted in FIGS. 2 and 7 of WO 02/24930, functional fragments thereof and sequences being at least 70% identical thereto while still retaining activity; sequences comprising SEQ ID NO: 28 of US 2005/181428, functional fragments thereof and sequences being at least 70% identical thereto while still retaining activity.

Preferably, said chromatin control element is an anti-repressor sequence, preferably chosen from the group consisting of: a) any one SEQ. ID. NO. 1 through SEQ. ID. NO. 66; b) fragments of any one of SEQ. ID. NO. 1 through SEQ. ID. NO. 66, wherein said fragments have anti-repressor activity (‘functional fragments’); c) sequences that are at least 70% identical in nucleotide sequence to a) or b) wherein said sequences have anti-repressor activity (‘functional derivatives’); and d) the complement to any one of a) to c). Preferably, said chromatin control element is chosen from the group consisting of STAR67 (SEQ. ID. NO. 66), STAR7 (SEQ. ID. NO. 7), STAR9 (SEQ. ID. NO. 9), STAR17 (SEQ. ID. NO. 17), STAR27 (SEQ. ID. NO. 27), STAR29 (SEQ. ID. NO. 29), STAR43 (SEQ. ID. NO. 43), STAR44 (SEQ. ID. NO. 44), STAR45 (SEQ. ID. NO. 45), STAR47 (SEQ. ID. NO. 47), STAR61 (SEQ. ID. NO. 61), or a functional fragment or derivative of said STAR sequences. In a particularly preferred embodiment, said STAR sequence is STAR 67 (SEQ. ID. NO. 66) or a functional fragment or derivative thereof. In certain preferred embodiments, STAR 67 or a functional fragment or derivative thereof is positioned upstream of a promoter driving expression of the multicistronic transcription unit. In other preferred embodiments, the expression cassettes according to the invention are flanked on both sides by at least one anti-repressor sequence.

Sequences having anti-repressor activity as used herein are sequences that are capable of at least in part counteracting the repressive effect of HP1 or HPC2 proteins when these proteins are tethered to DNA. Sequences having anti-repressor activity (sometimes also referred to as anti-repressor sequences or anti-repressor elements herein) suitable for the present invention, have been disclosed in WO 03/004704, incorporated herein by reference, and were coined “STAR” sequences therein (wherever a sequence is referred to as a STAR sequence herein, this sequence has anti-repressor activity according to the invention). As a non-limiting example, the sequences of 66 anti-repressor elements, named STAR1-65 (see WO 03/004704) and STAR67 (see WO 2006/005718), are presented herein as SEQ. ID. NOs. 1-65 and 66, respectively.

According to the invention, a functional fragment or derivative of a given anti-repressor element is considered equivalent to said anti-repressor element, when it still has anti-repressor activity. The presence of such anti-repressor activity can easily be checked by the person skilled in the art, for instance by the assay described below. Functional fragments or derivatives can easily be obtained by a person skilled in the art of molecular biology, by starting with a given anti-repressor sequence, and making deletions, additions, substitutions, inversions and the like (see e.g. WO 03/004704). A functional fragment or derivative also comprises orthologs from other species, which can be found using the known anti-repressor sequences by methods known by the person skilled in the art (see e.g. WO 03/004704). Hence, the present invention encompasses fragments of the anti-repressor sequences, wherein said fragments still have anti-repressor activity. The invention also encompasses sequences that are at least 70% identical in nucleotide sequence to said sequences having anti-repressor activity or to functional fragments thereof having anti-repressor activity, as long as these sequences that are at least 70% identical still have the anti-repressor activity according to the invention. Preferably, said sequences are at least 80% identical, more preferably at least 90% identical and still more preferably at least 95% identical to the reference native sequence or functional fragment thereof. For fragments of a given sequence, percent identity refers to that portion of the reference native sequence that is found in the fragment.

Sequences having anti-repressor activity according to the invention can be obtained by various methods, including but not limited to the cloning from the human genome or from the genome of another organism, or by for instance amplifying known anti-repressor sequences directly from such a genome by using the knowledge of the sequences, e.g. by PCR, or can in part or wholly be chemically synthesized.

Sequences having anti-repressor activity, and functional fragments or derivatives thereof, are structurally defined herein by their sequence and in addition are functionally defined as sequences having anti-repressor activity, which can be determined with the assay described below.

Any sequence having anti-repressor activity according to the present invention should at least be capable of surviving the following functional assay (see WO 03/004704, example 1, incorporated herein by reference).

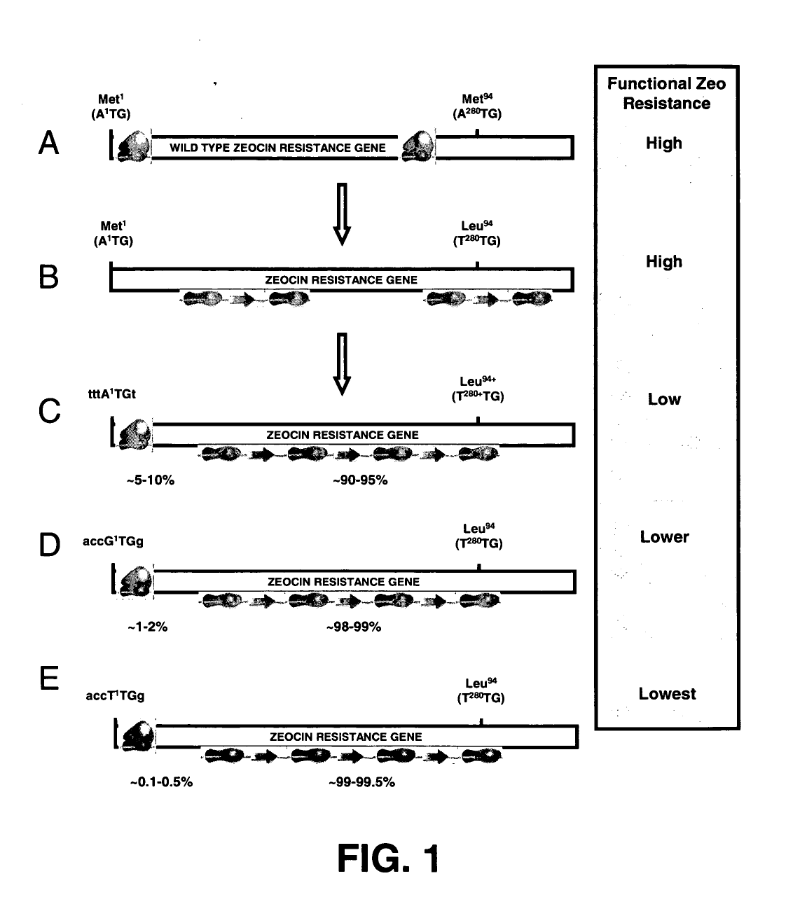

Human U-2 OS cells (ATCC HTB-96) are stably transfected with the pTet-Off plasmid (Clontech K1620-A) and with nucleic acid encoding a LexA-repressor fusion protein containing the LexA DNA binding domain and the coding region of either HP1 or HPC2 (Drosophila Polycomb group proteins that repress gene expression when tethered to DNA; the assay works with either fusion protein) under control of the Tet-Off transcriptional regulatory system (Gossen and Bujard, 1992). These cells are referred to below as the reporter cells for the anti-repressor activity assay. A reporter plasmid, which provides hygromycin resistance, contains a polylinker sequence positioned between four LexA operator sites and the SV40 promoter that controls the zeocin resistance gene. The sequence to be tested for anti-repressor activity can be cloned in said polylinker. Construction of a suitable reporter plasmid, such as pSelect, is described in example 1 and FIG. 1 of WO 00/004704. The reporter plasmid is transfected into the reporter cells, and the cells are cultured under hygromycin selection (25 μg/ml; selection for presence of the reporter plasmid) and tetracycline repression (doxycycline, 10 ng/ml; prevents expression of the LexA-repressor fusion protein). After 1 week of growth under these conditions, the doxycycline concentration is reduced to 0.1 ng/ml to induce the LexA-repressor gene, and after 2 days zeocin is added to 250 μg/ml. The cells are cultured for 5 weeks, until the control cultures (transfected with empty reporter plasmid, i.e. lacking a cloned anti-repressor sequence in the polylinker) are killed by the zeocin (in this control plasmid, the SV40 promoter is repressed by the LexA-repressor fusion protein that is tethered to the LexA operating sites, resulting in insufficient zeocin expression in such cells to survive zeocin selection). A sequence has anti-repressor activity according to the present invention if, when said sequence is cloned in the polylinker of the reporter plasmid, the reporter cells survive the 5 weeks selection under zeocin. Cells from such colonies can still be propagated onto new medium containing zeocin after the 5 weeks zeocin selection, whereas cells transfected with reporter plasmids lacking anti-repressor sequences cannot be propagated onto new medium containing zeocin. Any sequence not capable of conferring such growth after 5 weeks on zeocin in this assay, does not qualify as a sequence having anti-repressor activity, or functional fragment or functional derivative thereof according to the present invention. As an example, other known chromatin control elements such as those tested by Van der Vlag et al (2000), including Drosophila scs (Kellum and Schedl, 1991), 5′-HS4 of the chicken β-globin locus (Chung et al, 1993, 1997) or Matrix Attachment Regions (MARs) (Phi-Van et al., 1990), do not survive this assay.

In addition, it is preferred that the anti-repressor sequence or functional fragment or derivative thereof confers a higher proportion of reporter over-expressing clones when flanking a reporter gene (e.g. luciferase, GFP) which is integrated into the genome of U-2 OS or CHO cells, compared to when said reporter gene is not flanked by anti-repressor sequences, or flanked by weaker repression blocking sequences such as Drosophila scs. This can be verified using for instance the pSDH vector, or similar vectors, as described in example 1 and FIG. 2 of WO 03/004704.

Anti-repressor elements can have at least one of three consequences for production of protein: (1) they increase the predictability of identifying host cell lines that express a protein at industrially acceptable levels (they impair the ability of adjacent heterochromatin to silence the transgene, so that the position of integration has a less pronounced effect on expression); (2) they result in host cell lines with increased protein yields; and/or (3) they result in host cell lines that exhibit more stable protein production during prolonged cultivation.

Any STAR sequence can be used in the expression cassettes according to the present invention, but the following STAR sequences are particularly useful: STAR67 (SEQ. ID. NO. 66), STAR7 (SEQ. ID. NO. 7), STAR9 (SEQ. ID. NO. 9), STAR17 (SEQ. ID. NO. 17), STAR27 (SEQ. ID. NO. 27), STAR29 (SEQ. ID. NO. 29), STAR43 (SEQ. ID. NO. 43), STAR44 (SEQ. ID. NO. 44), STAR45 (SEQ. ID. NO. 45), STAR47 (SEQ. ID. NO. 47), STAR61 (SEQ. ID. NO. 61), or functional fragments or derivatives of these STAR sequences.

In certain embodiments said anti-repressor sequence, preferably STAR67, is placed upstream of said promoter, preferably such that less than 2 kb are present between the 3′ end of the anti-repressor sequence and the start of the promoter sequence. In preferred embodiments, less than 1 kb, more preferably less than 500 nucleotides (nt), still more preferably less than about 200, 100, 50, or 30 nt are present between the 3′ end of the anti-repressor sequence and the start of the promoter sequence. In certain preferred embodiments, the anti-repressor sequence is cloned directly upstream of the promoter, resulting in only about 0-20 nt between the 3′ end of the anti-repressor sequence and the start of the promoter sequence.

For the production of multimeric proteins, two or more expression cassettes can be used. Preferably, both expression cassettes are multicistronic expression cassettes according to the invention, each coding for a different selectable marker protein, so that selection for both expression cassettes is possible. This embodiment has proven to give good results, e.g. for the expression of the heavy and light chain of antibodies. It will be clear that both expression cassettes may be placed on one nucleic acid molecule or both may be present on a separate nucleic acid molecule, before they are introduced into host cells. An advantage of placing them on one nucleic acid molecule is that the two expression cassettes are present in a single predetermined ratio (e.g. 1:1) when introduced into host cells. On the other hand, when present on two different nucleic acid molecules, this allows the possibility to vary the molar ratio of the two expression cassettes when introducing them into host cells, which may be an advantage if the preferred molar ratio is different from 1:1 or when it is unknown beforehand what is the preferred molar ratio, so that variation thereof and empirically finding the optimum can easily be performed by the skilled person. According to the invention, preferably at least one of the expression cassettes, but more preferably each of them, comprises a chromatin control element, more preferably an anti-repressor sequence.

In another embodiment, the different subunits or parts of a multimeric protein are present on a single expression cassette.

Instead of or in addition to the presence of a STAR sequence placed upstream of a promoter in an expression cassette, it has proven highly beneficial to provide a STAR sequence on both sides of an expression cassette, such that expression cassette comprising the transgene is flanked by two STAR sequences, which in certain embodiments are essentially identical to each other.

It is shown herein that the combination of a first anti-repressor element upstream of a promoter and flanking the expression cassette by two other anti-repressor sequences provides superior results.

As at least some anti-repressor sequences can be directional (WO 00/004704), the anti-repressor sequences flanking the expression cassette (anti-repressor A and B) may beneficially placed in opposite direction with respect to each other, such that the 3′ end of each of these anti-repressor sequences is facing inwards to the expression cassette (and to each other). Hence, in preferred embodiments, the 5′ side of an anti-repressor element faces the DNA/chromatin of which the influence on the transgene is to be diminished by said anti-repressor element. For an anti-repressor sequence upstream of a promoter in an expression cassette, the 3′ end faces the promoter. The sequences of the anti-repressor elements in the sequence listing (SEQ. ID. NOs. 1-66) are given in 5′ to 3′ direction, unless otherwise indicated.

In certain embodiments, transcription units or expression cassettes according to the invention are provided, further comprising: a) a transcription pause (TRAP) sequence upstream of the promoter that drives transcription of the multicistronic transcription unit, said TRAP being in a 5′ to 3′ direction; or b) a TRAP sequence downstream of said open reading frame of the polypeptide of interest and preferably downstream of the transcription termination sequence of said multicistronic transcription unit, said TRAP being in a 3′ to 5′ orientation; or c) both a) and b); wherein a TRAP sequence is functionally defined as a sequence which when placed into a transcription unit, results in a reduced level of transcription in the nucleic acid present on the 3′ side of the TRAP when compared to the level of transcription observed in the nucleic acid on the 5′ side of the TRAP. Non-limiting examples of TRAP sequences are transcription termination and/or polyadenylation signals. One non-limiting example of a TRAP sequence is given in SEQ. ID. NO. 126. Examples of other TRAP sequences, methods to find these, and uses thereof have been described in WO 2004/055215.

DNA molecules comprising multicistronic transcription units and/or expression cassettes according to the present invention can be used for improving expression of nucleic acid, preferably in host cells. The terms “cell”/“host cell” and “cell line”/“host cell line” are respectively typically defined as a cell and homogeneous populations thereof that can be maintained in cell culture by methods known in the art, and that have the ability to express heterologous or homologous proteins.

Prokaryotic host cells can be used to propagate and/or perform genetic engineering with the DNA molecules of the invention, especially when present on plasmids capable of replicating in prokaryotic host cells such as bacteria.

A host cell according to the present invention preferably is a eukaryotic cell, more preferably a mammalian cell, such as a rodent cell or a human cell or fusion between different cells. In certain non-limiting embodiments, said host cell is a U-2 OS osteosarcoma, CHO (Chinese hamster ovary), HEK 293, HuNS-1 myeloma, WERI-Rb-1 retinoblastoma, BHK, Vero, non-secreting mouse myeloma Sp2/0-Ag 14, non-secreting mouse myeloma NS0, NCI-H295R adrenal gland carcinomal or a PER.C6 cell.

In certain embodiments of the invention, a host cell is a cell expressing at least E1A, and preferably also E1B, of an adenovirus. As non-limiting examples, such a cell can be derived from for instance human cells, for instance from a kidney (example: HEK 293 cells, see Graham et al, 1977), lung (e.g. A549, see e.g. WO 98/39411) or retina (example: HER cells marketed under the trade mark PER.C6™, see U.S. Pat. No. 5,994,128), or from amniocytes (e.g. N52.E6, described in U.S. Pat. No. 6,558,948), and similarly from other cells. Methods for obtaining such cells are described for instance in U.S. Pat. No. 5,994,128 and U.S. Pat. No. 6,558,948. PER.C6 cells for the purpose of the present invention means cells from an upstream or downstream passage or a descendent of an upstream or downstream passage of cells as deposited under ECACC no. 96022940, i.e. having the characteristics of those cells. It has been previously shown that such cells are capable of expression of proteins at high levels (e.g. WO 00/63403, and Jones et al, 2003). In other preferred embodiments, the host cells are CHO cells, for instance CHO-K1, CHO-S, CHO-DG44, CHO-DUKXB11, and the like. In certain embodiments, said CHO cells have a dhfr⁻ phenotype.

Such eukaryotic host cells can express desired polypeptides, and are often used for that purpose. They can be obtained by introduction of a DNA molecule of the invention, preferably in the form of an expression cassette, into the cells. Preferably, the expression cassette is integrated in the genome of the host cells, which can be in different positions in various host cells, and selection will provide for a clone where the transgene is integrated in a suitable position, leading to a host cell clone with desired properties in terms of expression levels, stability, growth characteristics, and the like. Alternatively the multicistronic transcription unit may be targeted or randomly selected for integration into a chromosomal region that is transcriptionally active, e.g. behind a promoter present in the genome. Selection for cells containing the DNA of the invention can be performed by selecting for the selectable marker polypeptide, using routine methods known by the person skilled in the art. When such a multicistronic transcription unit is integrated behind a promoter in the genome, an expression cassette according to the invention can be generated in situ, i.e. within the genome of the host cells.

Preferably the host cells are from a stable clone that can be selected and propagated according to standard procedures known to the person skilled in the art. A culture of such a clone is capable of producing polypeptide of interest, if the cells comprise the multicistronic transcription unit of the invention. Cells according to the invention preferably are able to grow in suspension culture in serum-free medium.

In preferred embodiments, the DNA molecule comprising the multicistronic transcription unit of the invention, preferably in the form of an expression cassette, is integrated into the genome of the eukaryotic host cell according to the invention. This will provide for stable inheritance of the multicistronic transcription unit.

Selection for the presence of the selectable marker polypeptide, and hence for expression, can be performed during the initial obtaining of the cells, and could be lowered or stopped altogether after stable clones have been obtained. It is however also possible to apply the selection agent during later stages continuously, or only occasionally, possibly at lower levels than during initial selection of the host cells.

A polypeptide of interest according to the invention can be any protein, and may be a monomeric protein or a (part of a) multimeric protein. A multimeric protein comprises at least two polypeptide chains. Non-limiting examples of a protein of interest according to the invention are enzymes, hormones, immunoglobulin chains, therapeutic proteins like anti-cancer proteins, blood coagulation proteins such as Factor VIII, multi-functional proteins, such as erythropoietin, diagnostic proteins, or proteins or fragments thereof useful for vaccination purposes, all known to the person skilled in the art.

In certain embodiments, an expression cassette of the invention encodes an immunoglobulin heavy or light chain or an antigen binding part, derivative and/or analogue thereof. In a preferred embodiment a protein expression unit according to the invention is provided, wherein said protein of interest is an immunoglobulin heavy chain. In yet another preferred embodiment a protein expression unit according to the invention is provided, wherein said protein of interest is an immunoglobulin light chain. When these two protein expression units are present within the same (host) cell a multimeric protein and more specifically an immunoglobulin, is assembled. Hence, in certain embodiments, the protein of interest is an immunoglobulin, such as an antibody, which is a multimeric protein. Preferably, such an antibody is a human or humanized antibody. In certain embodiments thereof, it is an IgG, IgA, or IgM antibody. An immunoglobulin may be encoded by the heavy and light chains on different expression cassettes, or on a single expression cassette. Preferably, the heavy and light chain are each present on a separate expression cassette, each having its own promoter (which may be the same or different for the two expression cassettes), each comprising a multicistronic transcription unit according to the invention, the heavy and light chain being the polypeptide of interest, and preferably each coding for a different selectable marker protein, so that selection for both heavy and light chain expression cassette can be performed when the expression cassettes are introduced and/or present in a eukaryotic host cell.

The polypeptide of interest may be from any source, and in certain embodiments is a mammalian protein, an artificial protein (e.g. a fusion protein or mutated protein), and preferably is a human protein.

Obviously, the configurations of the expression cassettes of the present invention may also be used when the ultimate goal is not the production of a polypeptide of interest, but the RNA itself, for instance for producing increased quantities of RNA from an expression cassette, which may be used for purposes of regulating other genes (e.g. RNAi, antisense RNA), gene therapy, in vitro protein production, etc.

In one aspect, the invention provides a method for generating a host cell expressing a polypeptide of interest, the method comprising the steps of: a) introducing into a plurality of precursor cells an expression cassette according to the invention, and b) culturing the generated cells under conditions selecting for expression of the selectable marker polypeptide, and c) selecting at least one host cell producing the polypeptide of interest. This novel method provides a very good result in terms of the ratio of obtained clones versus clones with high expression of the desired polypeptide. Using the most stringent conditions, i.e. the weakest translation efficiency for the selectable marker polypeptide (using the weakest translation start sequence), far fewer colonies are obtained using the same concentration of selection agent than with known selection systems, and a relatively high percentage of the obtained clones produces the polypeptide of interest at high levels. In addition, the obtained levels of expression appear higher than those obtained when an even larger number of clones using the known selection systems are used.

It is an additional advantage that the selection system is swift because it does not require copy number amplification of the transgene. Hence, cells with low copy numbers of the multicistronic transcription units already provide high expression levels. High transgene copy numbers of the transgene may be prone to genetic instability and repeat-induced silencing (e.g. Kim et al, 1998; McBurney et al, 2002). Therefore, an additional advantage of the embodiments of the invention with relatively low transgene copy numbers is that lower copy numbers are anticipated to be less prone to recombination and to repeat-induced silencing, and therefore less problems in this respect are anticipated when using host cells with a limited number of copies of the transgene compared to host cells obtained using an amplification system where hundreds or even thousands of copies of the selectable marker and protein of interest coding sequences may be present in the genome of the cell. The present invention provides examples of high expression levels, using the multicistronic transcription unit selection system, while the copy number of the transgene is relatively low, i.e less than 30 copies per cell, or even less than 20 copies per cell. Hence, the present invention allows the generation of host cells according to the invention, comprising less than 30 copies of the multicistronic transcription unit in the genome of the host cells, preferably less than 25, more preferably less than 20 copies, while at the same time providing sufficient expression levels of the polypeptide of interest for commercial purposes, e.g. more than 15, preferably more than 20 pg/cell/day of an antibody.

While clones having relatively low copy numbers of the multicistronic transcription units and high expression levels can be obtained, the selection system of the invention nevertheless can be combined with amplification methods to even further improve expression levels. This can for instance be accomplished by amplification of a co-integrated dhfr gene using methotrexate, for instance by placing dhfr on the same nucleic acid molecule as the multicistronic transcription unit of the invention, or by cotransfection when dhfr is on a separate DNA molecule.

In one aspect, the invention provides a method for producing a polypeptide of interest, the method comprising culturing a host cell, said host cell comprising a DNA molecule comprising a multicistronic expression unit or an expression cassette according to the invention, and expressing the polypeptide of interest from the coding sequence for the polypeptide of interest.

The host cell for this aspect is a eukaryotic host cell, preferably a mammalian cell, such as a CHO cell, further as described above.

Introduction of nucleic acid that is to be expressed in a cell, can be done by one of several methods, which as such are known to the person skilled in the art, also dependent on the format of the nucleic acid to be introduced. Said methods include but are not limited to transfection, infection, injection, transformation, and the like. Suitable host cells that express the polypeptide of interest can be obtained by selection as described above.

In certain embodiments, selection agent is present in the culture medium at least part of the time during the culturing, either in sufficient concentrations to select for cells expressing the selectable marker polypeptide or in lower concentrations. In preferred embodiments, selection agent is no longer present in the culture medium during the production phase when the polypeptide is expressed.

Culturing a cell is done to enable it to metabolize, and/or grow and/or divide and/or produce recombinant proteins of interest. This can be accomplished by methods well known to persons skilled in the art, and includes but is not limited to providing nutrients for the cell. The methods comprise growth adhering to surfaces, growth in suspension, or combinations thereof. Culturing can be done for instance in dishes, roller bottles or in bioreactors, using batch, fed-batch, continuous systems such as perfusion systems, and the like. In order to achieve large scale (continuous) production of recombinant proteins through cell culture it is preferred in the art to have cells capable of growing in suspension, and it is preferred to have cells capable of being cultured in the absence of animal- or human-derived serum or animal- or human-derived serum components.

The conditions for growing or multiplying cells (see e.g. Tissue Culture, Academic Press, Kruse and Paterson, editors (1973)) and the conditions for expression of the recombinant product are known to the person skilled in the art. In general, principles, protocols, and practical techniques for maximizing the productivity of mammalian cell cultures can be found in Mammalian Cell Biotechnology: a Practical Approach (M. Butler, ed., IRL Press, 1991).

In a preferred embodiment, the expressed protein is collected (isolated), either from the cells or from the culture medium or from both. It may then be further purified using known methods, e.g. filtration, column chromatography, etc, by methods generally known to the person skilled in the art.

The selection method according to the present invention works in the absence of chromatin control elements, but improved results are obtained when the multicistronic expression units are provided with such elements. The selection method according to the present invention works particularly well when an expression cassette according to the invention, comprising at least one anti-repressor sequence is used. Depending on the selection agent and conditions, the selection can in certain cases be made so stringent, that only very few or even no host cells survive the selection, unless anti-repressor sequences are present. Hence, the combination of the novel selection method and anti-repressor sequences provides a very attractive method to obtain only limited numbers of colonies with a greatly improved chance of high expression of the polypeptide of interest therein, while at the same time the obtained clones comprising the expression cassettes with anti-repressor sequences provide for stable expression of the polypeptide of interest, i.e. they are less prone to silencing or other mechanisms of lowering expression than conventional expression cassettes.

In certain embodiments, almost no clones are obtained when no anti-repressor sequence is present in the expression cassette according to the invention, providing for very stringent selection. The novel selection system disclosed herein therefore also provides the possibility to test parts of anti-repressor elements for functionality, by analyzing the effects of such sequences when present in expression cassettes of the invention under selection conditions. This easy screen, which provides an almost or even complete black and white difference in many cases, therefore can contribute to identifying functional parts or derivatives from anti-repressor sequences. When known anti-repressor sequences are tested, this assay can be used to characterize them further. When fragments of known anti-repressor sequences are tested, the assay will provide functional fragments of such known anti-repressor sequences.

In one aspect the invention provides a multicistronic transcription unit having an alternative configuration compared to the configuration disclosed in the incorporated '525 application: in the alternative configuration of the present invention, the sequence coding for the polypeptide of interest is upstream of the sequence coding for the selectable marker polypeptide, and the selectable marker polypeptide is operably linked to a cap-independent translation initiation sequence, preferably an internal ribosome entry site (IRES). Such multicistronic transcription units as such were known (e.g. Rees et al, 1996, WO 03/106684), but had not been combined with a non-optimal startcodon. According to the alternative of the present invention, the startcodon (or the context thereof) of the selectable marker polypeptide is changed into a non-optimal startcodon, to further decrease the translation initiation rate for the selectable marker. This therefore leads to a desired decreased level of expression of the selectable marker polypeptide, and can result in highly effective selection host cells expressing high levels of the polypeptide of interest, as with the embodiments disclosed in the incorporated '525 application. One potential advantage of this alternative aspect of the present invention, compared to the embodiments outlined in the '525 application, is that the coding sequence of the selectable marker polypeptide needs no further modification of internal ATG sequences, because any internal ATG sequences therein can remain intact since they are no longer relevant for translation of further downstream polypeptides. This may be especially advantageous if the coding sequence for the selectable marker polypeptide contains several internal ATG sequences, because the task of changing these and testing the resulting construct for functionality does not have to be performed for the present invention: only mutation of the ATG startcodon (or its context) suffices in this case. As will be understood by the person skilled in the art after reading the description of the present invention, this aspect of the invention can further be advantageously combined with the embodiments outlined above for the multicistronic transcription units. For instance expression cassettes comprising the multicistronic transcription unit can further in preferred embodiments comprise at least one chromatin control element. It is shown hereinbelow (example 19) that this alternative provided by the present invention also leads to very good results.

In one aspect, the invention therefore provides a DNA molecule comprising a multicistronic transcription unit coding for i) a polypeptide of interest, and for ii) a selectable marker polypeptide functional in a eukaryotic host cell, wherein the polypeptide of interest has a translation initiation sequence separate from that of the selectable marker polypeptide, and wherein the coding sequence for the polypeptide of interest is upstream from the coding sequence for the selectable marker polypeptide in said multicistronic transcription unit, and wherein an internal ribosome entry site (IRES) is present downstream from the coding sequence for the polypeptide of interest and upstream from the coding sequence for the selectable marker polypeptide, and wherein the nucleic acid sequence coding for the selectable marker polypeptide in the coding strand comprises a translation start sequence chosen from the group consisting of: a) an ATG startcodon in a non-optimal context for translation initiation, comprising the sequence (C/T)(A/T/G)(A/T/G)ATG(A/T/C) wherein the startcodon is underlined; b) a GTG startcodon; c) a TTG startcodon; d) a CTG startcodon; e) a ATT startcodon; and f) a ACG startcodon. The coding sequence for the selectable marker polypeptide is under translational control of the IRES, whereas the coding sequence for the protein of interest is preferably translated in a cap-dependent manner. The coding sequence for the polypeptide of interest comprises a stopcodon, so that translation of the first cistron ends upstream of the IRES, which IRES is operably linked to the second cistron.

As will be readily apparent to the skilled person after reading the present disclosure, most parts of these multicistronic expression units can be advantageously varied along the same lines as indicated above for the multicistronic expression units having an opposite order of the coding sequences for the polypeptide of interest and the selectable marker polypeptide (i.e. the multicistronic transcription units of the incorporated '525 application). For instance, the preferred startcodons for the selectable marker polypeptide, the incorporation into expression cassettes, the host cells, the promoters, the presence of chromatin control elements, etc. can be varied and used in preferred embodiments as described supra. Also the use of these multicistronic expression units and expression cassettes is as described supra. Therefore, this aspect is really an alternative to the means and methods described in the incorporated '525 application, with the main difference being that the order of the polypeptides in the multicistronic expression units is reversed, and that an IRES is now required for the translation of the selectable marker polypeptide.

As used herein, an “internal ribosome entry site” or “IRES” refers to an element that promotes direct internal ribosome entry to the initiation codon, such as normally an ATG, but in this invention preferably GTG or TTG, of a cistron (a protein encoding region), thereby leading to the cap-independent translation of the gene. See, e.g., Jackson R J, Howell M T, Kaminski A (1990) Trends Biochem Sci 15 (12): 477-83) and Jackson R J and Kaminski, A. (1995) RNA 1 (10): 985-1000. The present invention encompasses the use of any IRES element, which is able to promote direct internal ribosome entry to the initiation codon of a cistron. “Under translational control of an IRES” as used herein means that translation is associated with the IRES and proceeds in a cap-independent manner. As used herein, the term “IRES” encompasses functional variations of IRES sequences as long as the variation is able to promote direct internal ribosome entry to the initiation codon of a cistron. As used herein, “cistron” refers to a polynucleotide sequence, or gene, of a protein, polypeptide, or peptide of interest. “Operably linked” refers to a situation where the components described are in a relationship permitting them to function in their intended manner. Thus, for example, a promoter “operably linked” to a cistron is ligated in such a manner that expression of the cistron is achieved under conditions compatible with the promoter. Similarly, a nucleotide sequence of an IRES operably linked to a cistron is ligated in such a manner that translation of the cistron is achieved under conditions compatible with the IRES.

Internal ribosome binding site (IRES) elements are known from viral and mammalian genes (Martinez-Salas, 1999), and have also been identified in screens of small synthetic oligonucleotides (Venkatesan & Dasgupta, 2001). The IRES from the encephalomyocarditis virus has been analyzed in detail (Mizuguchi et al., 2000). An IRES is an element encoded in DNA that results in a structure in the transcribed RNA at which eukaryotic ribosomes can bind and initiate translation. An IRES permits two or more proteins to be produced from a single RNA molecule (the first protein is translated by ribosomes that bind the RNA at the cap structure of its 5′ terminus, (Martinez-Salas, 1999)). Translation of proteins from IRES elements is less efficient than cap-dependent translation: the amount of protein from IRES-dependent open reading frames (ORFs) ranges from less than 20% to 50% of the amount from the first ORF (Mizuguchi et al., 2000). The reduced efficiency of IRES-dependent translation provides an advantage that is exploited by this embodiment of the current invention. Furthermore, mutation of IRES elements can attenuate their activity, and lower the expression from the IRES-dependent ORFs to below 10% of the first ORF (Lopez de Quinto & Martinez-Salas, 1998, Rees et al., 1996). The advantage exploited by the invention is as follows: when the IRES-dependent ORF encodes a selectable marker protein, its low relative level of translation means that high absolute levels of transcription must occur in order for the recombinant host cell to be selected. Therefore, selected recombinant host cell isolates will by necessity express high amounts of the transgene mRNA. Since the recombinant protein is translated from the cap-dependent ORF, it can be produced in abundance resulting in high product yields. On top of this, the non-optimal (i.e. non-ATG) startcodon for the selectable marker polypeptide according to the invention, further improves the chances of obtaining a preferred host cell, i.e. a host cell expressing high levels of recombinant protein of interest.

It is clear to a person skilled in the art that changes to the IRES can be made without altering the essence of the function of the IRES (hence, providing a protein translation initiation site with a reduced translation efficiency), resulting in a modified IRES. Use of a modified IRES which is still capable of providing a small percentage of translation (compared to a 5′ cap translation) is therefore also included in this invention.

The practice of this invention will employ, unless otherwise indicated, conventional techniques of immunology, molecular biology, microbiology, cell biology, and recombinant DNA, which are within the skill of the art. See e.g. Sambrook, Fritsch and Maniatis, Molecular Cloning: A Laboratory Manual, 2^{nd} edition, 1989; Current Protocols in Molecular Biology, Ausubel F M, et al, eds, 1987; the series Methods in Enzymology (Academic Press, Inc.); PCR2: A Practical Approach, MacPherson M J, Hams B D, Taylor G R, eds, 1995; Antibodies: A Laboratory Manual, Harlow and Lane, eds, 1988.

The invention is further explained in the following examples. The examples do not limit the invention in any way. They merely serve to clarify the invention.

### EXAMPLES

Examples 1-18 describe details of several embodiments of the incorporated '525 application. Example 19 describes the selection system with the multicistronic transcription unit of the present invention, and it will be clear that the variations described in examples 1-18 can also be applied and tested for the multicistronic transcription units of the present application.

### Example 1

### Construction and Testing of a Zeocin Resistance Gene Product with No Internal Methionine

The basic idea behind the development of the novel selection system of the incorporated '525 application is to place the gene encoding the resistance gene upstream of a gene of interest, and one promoter drives the expression of this bicistronic mRNA. The translation of the bicistronic mRNA is such that only in a small percentage of translation events the resistance gene will be translated into protein and that most of the time the downstream gene of interest will be translated into protein. Hence the translation efficiency of the upstream resistance gene must be severely hampered in comparison to the translation efficiency of the downstream gene of interest. To achieve this, three steps can be taken according to the invention of the '525 application:

1) within the resistance gene on the mRNA, the searching ribosome preferably should not meet another AUG, since any downstream AUG may serve as translation start codon, resulting in a lower translation efficiency of the second, downstream gene of interest. Hence, preferably any AUG in the resistance gene mRNA will have to be replaced. In case this AUG is a functional codon that encodes a methionine, this amino acid will have to be replaced by a different amino acid, for instance by a leucine (FIGS. 1A and B);

2) the start codon of the resistance gene must have a bad context (be part of a non-optimal translation start sequence); i.e. the ribosomes must start translation at this start codon only in a limited number of events, and hence in most events continue to search for a better, more optimal start codon (FIG. 1C-E). Three different stringencies can be distinguished: a) the normal ATG startcodon, but placed in a bad context (TTTATGT) (called ATGmut) (FIG. 1C), b) preferably when placed in an optimal context, GTG can serve as startcodon (ACCGTGG) (FIG. 1D) and c) preferably when placed in an optimal context, TTG can serve as startcodon (ACCTTGG) (FIG. 1E). The most stringent translation condition is the TTG codon, followed by the GTG codon (FIG. 1). The Zeo mRNA with a TTG as start codon is expected to produce the least Zeocin resistance protein and will hence convey the lowest functional Zeocin resistance to cells (FIGS. 1, 2).

3) preferably, the normal start codon (ATG) of the downstream gene of interest should have an optimal translation context (e.g. ACCATGG)(FIG. 2A-D). This warrants that, after steps 1 and 2 have been taken, in most events the start codon of the gene of interest will function as start codon of the bicistronic mRNA.

In this example, step 1 is performed, that is, in the Zeocin resistance gene one existing internal methionine is replaced by another amino acid (FIG. 1B-E). It is important that after such a change the Zeo protein still confers Zeocin resistance to the transfected cells. Since it is not known beforehand which amino acid will fulfill this criterium, three different amino acids have been tried: leucine, threonine and valine. The different constructs with distinct amino acids have than been tested for their ability to still confer Zeocin resistance to the transfected cells.

Materials and Methods

Construction of the Plasmids

The original Zeo open reading frame has the following sequence around the startcodon: AAACCATGGCC (startcodon in bold; SEQ. ID. NO. 67). This is a startcodon with an optimal translational context (FIG. 1A). First the optimal context of the start codon of the Zeo open reading frame was changed through amplification from plasmid pCMV-zeo [Invitrogen V50120], with primer pair ZEOforwardMUT (SEQ. ID. NO. 68): GATCTCGCGATACAGGATTTATGTTGGCCAAGTTGACCAGTGCCGTTCCG and ZEO-WTreverse (WT=Wild type; SEQ. ID. NO. 69): AGGCGAATTCAGTCCTGCTCCTCGGC, using pCMV-ZEO (Invitrogen; V50120) as a template. The amplified product was cut with NruI-EcoRI, and ligated into pcDNA3, resulting in pZEOATGmut.

The original Zeo open reading frame contains an in frame ATG, encoding methionine at amino acid position 94 (out of 124). This internal ATG, encoding the methionine at position 94 was changed in such a way that the methionine was changed into leucine, threonine or valine respectively:

1) To replace the internal codon for methionine in the Zeo open reading frame with the codon for leucine (FIG. 1B), part of the Zeo open reading frame was amplified using primer pair ZEOforwardMUT (SEQ. ID. NO. 68) and ZEO-LEUreverse (SEQ. ID. NO. 70): AGGCCCCGCCCCCACGGCTGCTCGCCGATCTCGGTCAAGGCCGGC. The PCR product was cut with BamHI-BglI and ligated into pZEOATGmut. This resulted in pZEO(leu). To replace the internal codon for methionine in the Zeo open reading frame with the codon for threonine (not shown, but as in FIG. 1B), part of the Zeo open reading frame was amplified using primer pair ZEOforwardMUT (SEQ. ID. NO. 68) and ZEO-THRreverse (SEQ. ID. NO. 71): AGGCCCCGCCCCCACGGCTGCTCGCCGATCTCGGTGGTGGCCGGC. The PCR product was cut with BamHI-BglI and ligated into pZEOATGmut. This resulted in pZEO(thr). To replace the internal codon for methionine in the Zeo open reading frame with the codon for valine (not shown, but as in FIG. 1B)(GTG), part of the Zeo open reading frame was amplified using primer pair ZEOforwardMUT (SEQ. ID. NO. 68) and ZEO-VALreverse (SEQ. ID. NO. 72): AGGCCCCGCCCCCACGGCTGCTCGCCGATCTCGGTCCACGCCGG. The PCR product was cut with BamHI-BglI and ligated into pZEOATGmut. This resulted in pZEO(val).

Transfection and Culturing of Cells

The Chinese Hamster Ovary cell line CHO-K1 (ATCC CCL-61) was cultured in HAMS-F12 medium+10% Fetal Calf Serum containing 2 mM glutamine, 100 U/ml penicillin, and 100 micrograms/ml streptomycin at 37° C./5% CO₂. Cells were transfected with the plasmids using Lipofectamine 2000 (Invitrogen) as described by the manufacturer. Briefly, cells were seeded to culture vessels and grown overnight to 70-90% confluence. Lipofectamine reagent was combined with plasmid DNA at a ratio of 6 microliters per microgram (e.g. for a 10 cm Petri dish, 20 micrograms DNA and 120 microliters Lipofectamine) and added to the cells. After overnight incubation the transfection mixture was replaced with fresh medium, and the transfected cells were incubated further. After overnight cultivation, cells were trypsinized and seeded into fresh culture vessels with fresh medium containing zeocin (100 μg/ml). When individual colonies became visible (approximately ten days after transfection) colonies were counted.

Results

Four plasmids were transfected to CHO-K1 cells, 1) pZEO(WT), 2) pZEO(leu), 3) pZEO(thr), and 4) pZEO(val). The cells were selected on 100 μg/ml zeocine. Transfection of pZEO(leu) resulted in an equal number of zeocin resistant colonies in comparison with the control pZEO (WT). pZEO(thr) and pZEO(val) gave less colonies, but the differences were not in the order of a magnitude. Hence it was concluded that changes of the internal methionine into leucine, threonine or valine all resulted in a Zeocin resistance protein that is still able to confer zeocin resistance to the transfected cells. Rather arbitrarily, pZEO(leu) was chosen as starting point for creating different startcodons on the Zeo open reading frame. Hence in the examples below the start as well as internal methionines are always replaced by leucine, for zeocin, but also for other selectable marker genes, as will be clear from further examples.

### Example 2

### Creation and Testing of Zeocin-d2EGFP Bicistronic Constructs with Differential Translation Efficiencies

To create a bicistronic mRNA encompassing a mutated Zeocin resistance mRNA with less translational efficiency, and the d2EGFP gene as downstream gene of interest, the start codon of the d2EGFP gene was first optimized (step 3 in example 1). After that, the different versions of the Zeocin resistance gene were created. The differences between these versions are that they have different start codons, with distinct translational efficiency (step 2 in Example 1, FIG. 1C-E). These different Zeocin resistance gene versions were cloned upstream of the modified d2EGFP gene (FIG. 2).

Materials and Methods

Creation of Plasmids

The d2EGFP reporter ORF was introduced into pcDNA3. The sequence around the startcodon of this d2EGFP cDNA is GAATTCATGGG (startcodon in bold; SEQ. ID. NO. 73), which is not optimal. As a first step, d2EGFP was amplified from pd2EGFP (Clontech 6010-1) with primers d2EGFPforwardBamHI (SEQ. ID. NO. 74): GATCGGATCCTATGAGGAATTCGCCACCATGGTGAGCAAGGGCGAGGAG and d2EGFPreverseNotI (SEQ. ID. NO. 75): AAGGAAAAAAGCGGCCGCCTACACATTGATCCTAGCAGAAG. This product contains now a startcodon with an optimal translational context (ACCATGG). This created pd2EGFP and subsequently, the Zeo open reading frame was ligated into pd2EGFP, resulting in pZEO-d2EGFP. It is pointed out here that the optimization of the translational start sequence of the gene of interest (here: EGFP as a model gene) is not essential but preferred in order to skew the translation initiation frequency towards the gene of interest still further.

Now three classes of constructs were made:

1) ATG as a start codon in the Zeo resistance gene, but in a bad context (TTTATGT) (not shown, but as in FIG. 2B) and followed by spacer sequence, instead of the optimal ATG (FIG. 2A). The spacer sequence is placed downstream of the ATG sequence. In the zeocin (and possibly in the blasticidin) RNA, a secondary structure is present, causing the ribosome to be temporarily delayed. Because of this, a poor startcodon can in some cases be used by the ribosome, despite being a bad startcodon or being in a non-optimal context for translation initiation. This causes the chance of translation to increase, and in case of the current invention therefore renders the stringency for selection lower. To decrease this effect, and hence to further decrease the translation initation efficiency, a spacer sequence is introduced that does not contain a secondary structure (Kozak, 1990). Hence, the term ‘space’ is introduced, and used in the plasmid and primer names to indicate the presence of such a spacer sequence. The spacer removes the ‘ribosome delaying sequence’ from the neighbourhoud of the initiation codon, therewith causing the ribosome to start translating less frequently, and hence increasing the stringency of the selection according to the invention. The spacer introduces some extra amino acids in the coding sequence. This has been done in some cases for both zeocin and for blasticidin, as will be apparent from the examples. The nomenclature of the plasmids and primers in general in the following is along these lines: the name of the selectable marker polypeptide is referred to by abbreviation (e.g. Zeo, Blas, etc); the startcodon is mentioned (e.g. ATG, GTG, TTG); when this startcodon is placed in a non-optimal context for translation initiation, the addition “mut” is used (this is usually only done for ATG startcodons, as combining a non-optimal context with a non-ATG startcodon usually does not result in sufficient translation initiation to allow for selection); when a spacer sequence is used behind the startcodon, the addition “space” is used (this is done usually for “ATGmut” startcodons for Zeo or Blas selectable markers). The Zeo open reading frame was amplified with primer pair ZEOforwardBamHI-ATGmut/space (SEQ. ID. NO. 77): GATCGGATCCTTGGTTTATGTCGATCCAAAGACTGCCAAATCTAGATCCGAGATTTTC AGGAGCTAAGGAAGCTAAAGCCAAGTTGACCAGTGAAGTTC (wherein the sequence following the underlined sequence comprises the spacer sequence), and ZEOWTreverse (SEQ. ID. NO. 69), the PCR product was cut with EcoRI-BamHI, and ligated into pd2EGF, cut with EcoRI-BamHI, creating pZEO-ATGmut/space-d2EGFP.

2) GTG as a start codon in the Zeo resistance gene, instead of ATG (FIG. 2C). The Zeo open reading frame was amplified with primer pair ZEOforwardBamHI-GTG (SEQ. ID. NO. 78): GATCGGATCCACCGTGGCCAAGTTGACCAGTGCCGTTC and ZEOWTreverse (SEQ. ID. NO. 69), the PCR product was cut with EcoRI-BamHI, and ligated into pd2EGFP, cut with EcoRI-BamHI, creating pZEO-GTG-d2EGFP.

3) TTG as a start codon in the Zeo resistance gene, instead of ATG (FIG. 2D). The Zeo open reading frame was amplified with primer pair ZEOforwardBamHI-TTG: GATCGGATCCACCTTGGCCAAGTTGACCAGTGCCGTTC (SEQ. ID. NO. 79) and ZEOWTreverse (SEQ. ID. NO. 69), the PCR product was cut with EcoRI-BamHI, and ligated into pd2EGFP, cut with EcoRI-BamHI, creating pZEO-TTG-d2EGFP.

Transfection, Culturing and Analysis of CHO Cells

The Chinese Hamster Ovary cell line CHO-K1 (ATCC CCL-61) was cultured in HAMS-F12 medium+10% Fetal Calf Serum containing 2 mM glutamine, 100 U/ml penicillin, and 100 micrograms/ml streptomycin at 37° C./5% CO₂. Cells were transfected with the plasmids using Lipofectamine 2000 (Invitrogen) as described by the manufacturer. Briefly, cells were seeded to culture vessels and grown overnight to 70-90% confluence. Lipofectamine reagent was combined with plasmid DNA at a ratio of 15 microliters per 3 microgram (e.g. for a 10 cm Petri dish, 20 micrograms DNA and 120 microliters Lipofectamine) and added after 30 minutes incubation at 25° C. to the cells. After overnight incubation the transfection mixture was replaced with fresh medium, and the transfected cells were incubated further. After overnight cultivation, cells were trypsinized and seeded into fresh culture vessels with fresh medium. After another overnight incubation zeocin was added to a concentration of 50 μg/ml and the cells were cultured further. After another three days the medium was replaced by fresh medium containing zeocin (100 μg/ml) and cultured further. When individual colonies became visible (approximately ten days after transfection) medium was removed and replaced with fresh medium without zeocin. Individual clones were isolated and transferred to 24-well plates in medium without zeocin. One day after isolation of the colonies zeocin was added to the medium. Expression of the d2EGFP reporter gene was assessed approximately 3 weeks after transfection. d2EGFP expression levels in the colonies were measured after periods of two weeks.

Results

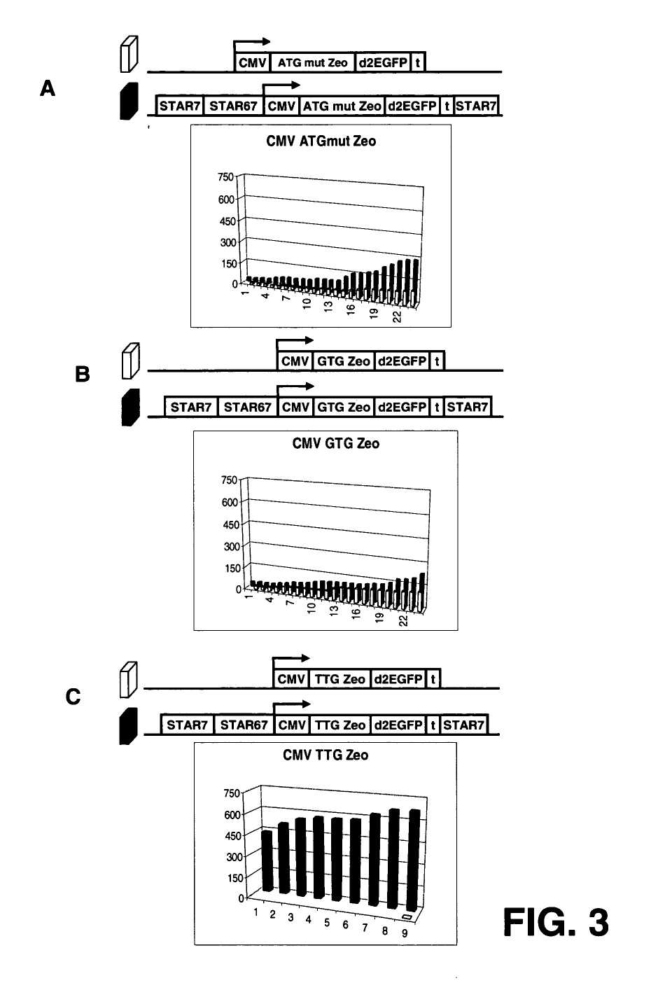

CHO-K1 cells were transfected with constructs that contain the ATGmut/space Zeo (FIG. 2B), GTG Zeo (FIG. 2C) and TTG Zeo (FIG. 2D) genes as selection gene, all being cloned upstream of the d2EGFP reporter gene. These three constructs were without STAR elements (Control) or with STAR elements 7 and 67 upstream of the CMV promoter and STAR 7 downstream from the d2EGFP gene (FIG. 3). FIG. 3 shows that both the control (without STAR elements) constructs with ATGmut/space Zeo (A) and GTG Zeo (B) gave colonies that expressed d2EGFP protein. The average d2EGFP expression level of 24 ATGmut/space Zeo colonies was 46 and of GTG Zeo colonies was 75. This higher average expression level in GTG Zeo colonies may reflect the higher stringency of GTG, in comparison with ATGmut/space (example 1). Addition of STAR elements 7 and 67 to the constructs resulted in colonies that had higher average d2EGFP expression levels. Transfection of the ATGmut/space Zeo STAR 7/67/7 construct resulted in colonies with an average d2EGFP expression level of 118, which is a factor 2.6 higher than the average in the control cells (46). Addition of STAR elements to the GTG Zeo construct resulted in an average d2EGFP expression level of 99, which is a factor 1.3 higher than the average in the control cells (75).

Importantly, no colonies were established when the TTG Zeo construct was transfected. However, the construct with TTG Zeo, flanked with STARs 7 and 67 resulted in the establishment of 6 colonies, with an average d2EGFP expression level of 576 (FIG. 3C). Thus the highest translation stringency, brought about by the TTG startcodon (FIG. 1) yields to the highest d2EGFP expression levels, as predicted in FIG. 2. The results also indicate that the stringency of the TTG Zeo alone (without STAR elements) is at least in some experiments too high for colonies to survive. However, in later independent experiments (see below), some colonies were found with this construct without STAR elements, indicating that the stringency of the selection system with the TTG startcodon in the zeocin selection marker not necessarily precludes the finding of colonies when no STAR elements are present, and that the number of colonies obtained may vary between experiments.

It is concluded that the use of STAR elements in combination with the stringent selection system according to the invention allows to readily identify high producers of the gene of interest.

### Example 3

### Establishment of a Higher Number of TTG Zeo STAR Colonies and Comparison with an IRES-Zeo Construct

The results in example 2 indicate that the TTG Zeo has extremely stringent translation efficiency, which might be to high to convey Zeocin resistance to the cells. The transfection was scaled up to test whether there would be some colonies that have such high expression levels that they survive. Scaling up the experiment could also address the question whether the high average of TTG Zeo STAR 7/67/7 would become higher when more colonies were analyzed.

Materials and Methods

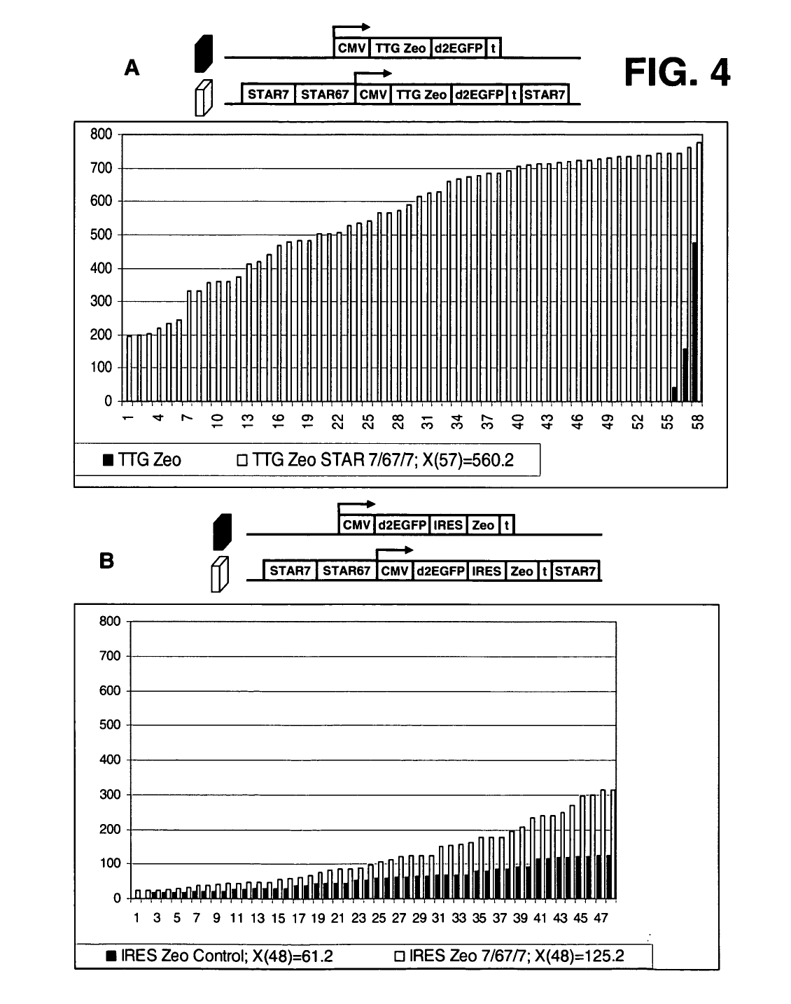

CHO-K1 cells were transfected with the constructs that have the TTG Zeo gene as selection marker, with and without STAR elements 7 and 67 (FIG. 4). Transfections, selection, culturing etc were as in example 2, except that 6 times more cells, DNA and Lipofectamine 2000 were used. Transfections and selection were done in Petri dishes.

Results

FIG. 4A shows that transfection with the TTG Zeo STAR 7/67/7 construct resulted in the generation of many colonies with an average d2EGFP signal of 560. This is as high as in example 2, except that now 58 colonies were analyzed. When compared to a construct with the Zeocin resistance gene placed behind an IRES sequence (FIG. 4B), the average d2EGFP expression level was 61, and when STAR elements 7 and 67 were added to such a construct, the average d2EGFP expression level was 125, a factor 2 above the control (FIG. 4B). The average of the TTG Zeo STAR 7/67/7 colonies was therefore a factor 9.2 higher than the STAR-less IRES-Zeo colonies and a factor 4.5 higher than the STAR7/67/7 IRES Zeo colonies.

An observation is that the form of the curve of all expressing colonies differs between the TTG Zeo STAR7/67/7 and IRES-Zeo STAR 7/67/7. In the first case (TTG Zeo) the curve levels off, whereas in the second case (IRES-Zeo) the curve has a more ‘exponential’ shape. The plateau in the TTG Zeo curve could indicate that the cells have reached a maximum d2EGFP expression level, above which the d2EGFP expression levels become toxic and the cells die. However, it later appeared that the high values were close to the maximum value that could be detected with the settings of the detector of the FACS analyser. In later experiments, the settings of the FACS analyser were changed to allow for detection of higher values, and indeed in some instances higher values than obtained here were measured in later independent experiments (see below).

Due to up-scaling of the transfections three colonies with the STAR-less TTG Zeo construct could be picked. The d2EGFP expression levels of these colonies were 475, 158 and 43. The last colony died soon after the first measurement. This result indicates that the TTG Zeo construct can convey Zeocin resistance, resulting in colonies that also can give high expression levels in some instances. Hence, the novel selection method according to the invention can be applied with expression cassettes that do not contain chromatin control elements, although it is clearly preferred to use expression cassettes comprising at least one such element, preferably a STAR element.

The results indicate that STAR elements allow a more stringent selection system according to the invention, such as exemplified in this example, resulting in the picking of colonies that have a very high average protein expression level.

### Example 4

### Creation and Testing of Blasticidin-d2EGFP Bicistronic Constructs with Differential Translation Efficiencies

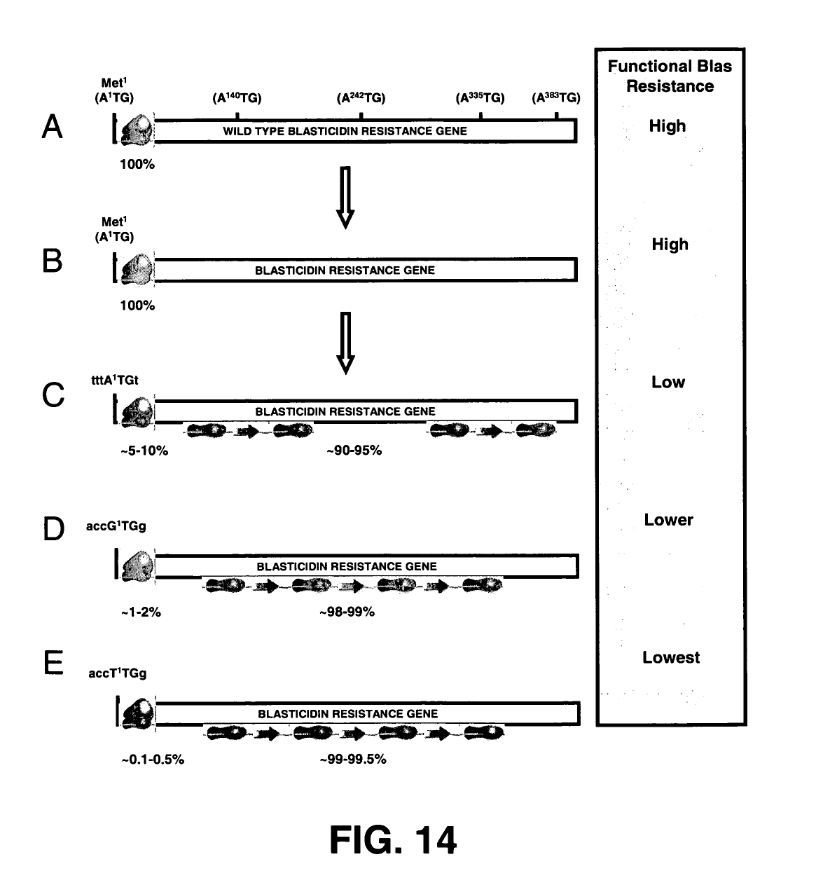

There are four internal ATGs in the blasticidine resistance gene, none of which codes for a methionine (FIG. 14A). These ATGs have to be eliminated though (FIG. 14B), since they will serve as start codon when the ATG startcodon (or the context thereof) has been modified, and this will result in peptides that do not resemble blasticidine resistance protein. More importantly, these ATGs will prevent efficient translation of the gene of interest, as represented by d2EGFP in this example for purposes of illustration. To eliminate the internal ATGs, the blasticidine resistance protein open reading frame was first amplified with 4 primer pairs, generating 4 blasticidine resistance protein fragments. The primer pairs were:

| | |
|---|---|
| A) | |
| BSDBamHIforward: | |
| GATCGGATCCACCATGGCCAAGCCTTTGTCTCAAG | (SEQ.ID.NO.80) |
| BSD150reverse: | |
| GTAAAATGATATACGTTGACACCAG | (SEQ.ID.NO.81) |
| B) | |
| BSD150forward: | |
| CTGGTGTCAACGTATATCATTTTAC | (SEQ.ID.NO.82) |
| BSD250reverse: | |
| GCCCTGTTCTCGTTTCCGATCGCG | (SEQ.ID.NO.83) |
| C) | |
| BSD250forward: | |
| CGCGATCGGAAACGAGAACAGGGC | (SEQ.ID.NO.84) |
| BSD350reverse: | |
| GCCGTCGGCTGTCCGTCACTGTCC | (SEQ.ID.NO.85) |
| D) | |
| BSD350forward: | |
| GGACAGTGACGGACAGCCGACGGC | (SEQ.ID.NO.86) |
| BSD399reverse: | |
| GATCGAATTCTTAGCCCTCCCACACGTAACCA | (SEQ.ID.NO.87) |
| GAGGGC | |

Fragments A to D were isolated from an agarose gel and mixed together. Next, only primers BSDBamHIforward and BSD399reverse were used to create the full length blasticidine resistance protein cDNA, but with all internal ATGs replaced. The reconstituted blasticidine was then cut with EcoRI-BamHI, and cloned into pZEO-GTG-d2EGFP, cut with EcoRI-BamHI (which releases Zeo), resulting in pBSDmut-d2EGFP. The entire blasticidine resistance protein open reading frame was sequenced to verify that all ATGs were replaced.

With this mutated gene encoding blasticidine resistance protein (Blas), three classes of constructs are made (FIG. 14C-E):

1) ATG as a start codon, but in a bad context and followed by spacer sequence. The mutated blasticidine resistance protein open reading frame in pBSD-d2EGFP was amplified using primers BSDforwardBamHIAvrII-ATGmut/space (SEQ. ID. NO. 88): GATCGGATCCTAGGTTGGTTTATGTCGATCCAAAGACTGCCAAATCTAGATCCGAGA TTTTCAGGAGCTAAGGAAGCTAAAGCCAAGCCTTTGTCTCAAGAAG,

and BSD399reverseEcoRIAvrII (SEQ. ID. NO. 89):

GATCGAATTCCCTAGGTTAGCCCTCCCACACGTAACCAGAGGGC, the PCR product is cut with BamHI-EcoRI, and ligated into pZEO-GTG-d2EGFP, cut with EcoRI-BamHI. This results in pBSD-ATGmut/space-d2EGFP.

2) GTG as a start codon instead of ATG. The mutated blasticidine resistance protein open reading frame in pBSD-d2EGFP was amplified using primers BSDforwardBamHIAvrII-GTG (SEQ. ID. NO. 90): GATCGGATCCTAGGACCGTGGCCAAGCCTTTGTCTCAAGAAG and BSD399reverseEcoRIAvrII (SEQ. ID. NO. 89), the PCR product was cut with BamHI-EcoRI, and ligated into pZEO-GTG-d2EGFP, cut with EcoRI-BamHI. This results in pBSD-GTG-d2EGFP.

3) TTG as a start codon instead of ATG. The mutated blasticidine open reading frame in pBSD-d2EGFP was amplified using primers BSDforwardBamHIAvrII-TTG (SEQ. ID. NO. 91): GATCGGATCCTAGGACCTTGGCCAAGCCTTTGTCTCAAGAAG and BSD399reverseEcoRIAvrII (SEQ. ID. NO. 89), the PCR product was cut with BamHI-EcoRI, and ligated into pZEO-GTG-d2EGFP, cut with EcoRI-BamHI. This results in pBSD-TTG-d2EGFP.

Results

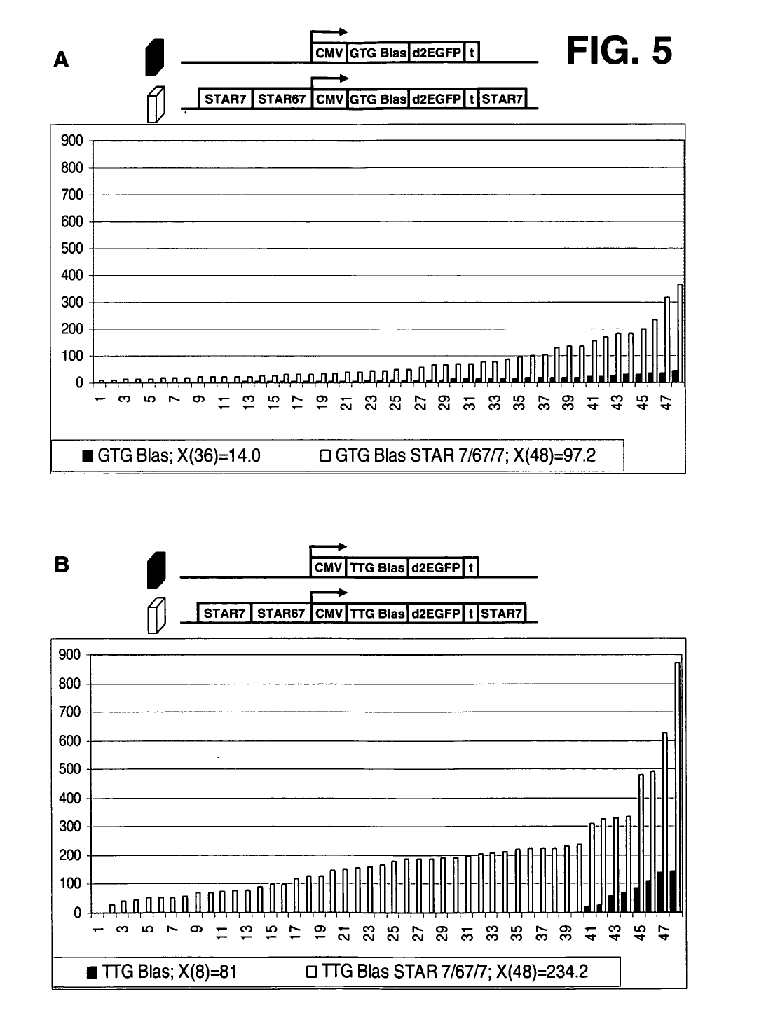

CHO-K1 cells were transfected with constructs that contain the GTG Blas (FIG. 5A) and TTG Blas (FIG. 5B) genes as selection gene, all being cloned upstream of the d2EGFP reporter gene. Selection took place in the presence of 20 μg/ml Blasticidine. The two constructs were without STAR elements (Control) or with STAR elements 7 and 67 upstream of the CMV promoter and STAR7 downstream from the d2EGFP gene (FIG. 5). FIG. 5 shows that both the control (without STAR elements) constructs with GTG Blas (A) and TTG Blas (B) gave colonies that expressed d2EGFP protein. The average d2EGFP signal of 24 GTG Blas colonies was 14.0 (FIG. 5A) and of TTG Blas colonies was 81 (FIG. 5B). This higher average expression level in TTG Blas colonies may reflect the higher stringency of TTG, in comparison with GTG (see also example 2). However, only 8 colonies survived under the more stringent TTG conditions.

Addition of STAR elements 7 and 67 to the constructs resulted in colonies that had higher average d2EGFP expression levels. Transfection of the GTG Blas STAR 7/67/7 construct resulted in colonies with an average d2EGFP expression level of 97.2 (FIG. 5A), which is a factor 6.9 higher than the average in the control cells (14.0). Addition of STAR elements to the TTG Blas construct resulted in an average d2EGFP signal of 234.2 (FIG. 5B), which is a factor 2.9 higher than the average in the control cells (81). However, note again that only 8 colonies survived the harsh selection conditions of TTG Blas, whereas 48 colonies survived with TTG Blas STAR 7/67/7. When only the five highest values are compared, the average of the five highest TTG Blas was 109.1 and the average of the five highest TTG Blas STAR 7/67/7 was 561.2, which is a factor 5.1 higher.

The results indicate that STAR elements allow a more stringent selection system, resulting in the picking of colonies that have a very high average protein expression level. They also show that this selection is not restricted to the Zeocin resistance protein alone, but that also other selection marker polypeptides, in this case the blasticidine resistance protein, can be used.

### Example 5

### Stability of d2EGFP Expression in the Novel Selection System

Colonies described in example 3 were further cultured under several conditions to assess the stability of d2EGFP expression over an extended time period.

Results

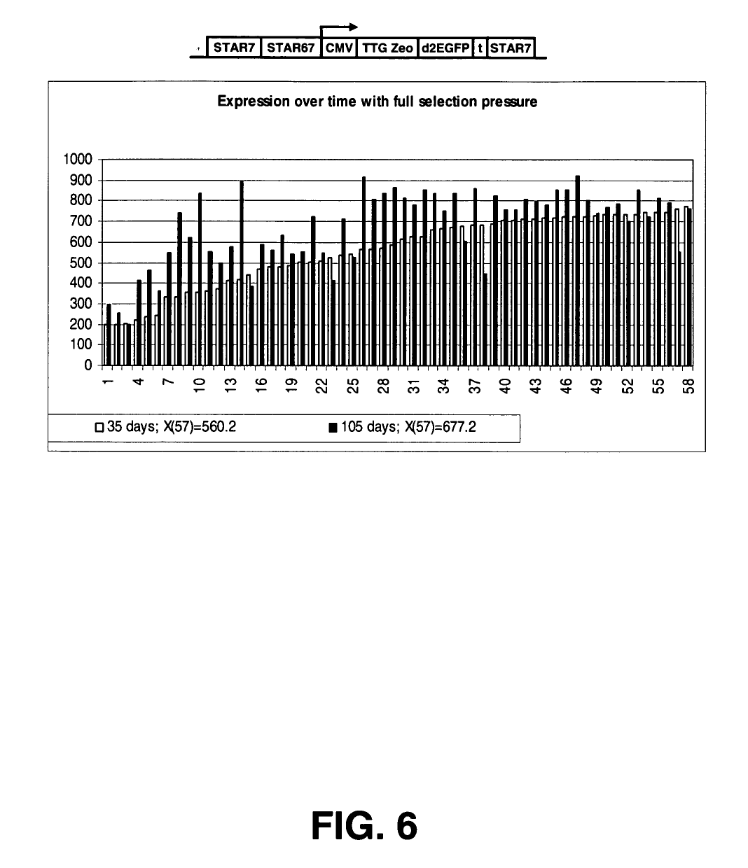

The TTG Zeo STAR 7/67/7 containing colonies in FIG. 4A were cultured for an additional 70 days in the presence of 100 μg/ml Zeocin. As shown in FIG. 6, the average d2EGFP signal rose from 560.2 after 35 days to 677.2 after 105 days. Except for some rare colonies all colonies had a higher d2EGFP expression level.

When the level of Zeocin was lowered to 20 μg/ml Zeocin, there was still an increase in the average d2EGFP expression level, from 560.2 after 35 days to 604.5 after 105 days (FIG. 7).

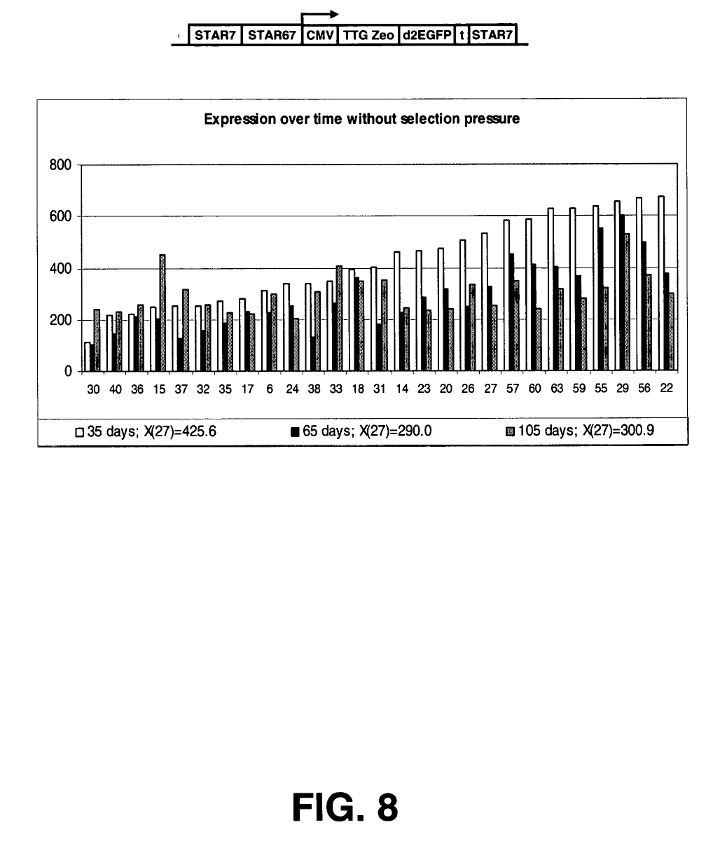

When no selection pressure was present at all due to removal of the Zeocin from the culture medium, approximately 50% of the colonies became mosaic, that is, within one colony non-d2EGFP expressing cells became apparent. This resulted in lowering of d2EGFP expression levels to less than 50% of the original levels. If the signal became less than 67% (decrease of at least one-third) from the original signal, the colony was considered to be unstable in respect to d2EGFP expression. Of the 57 original colonies 27 colonies remained stable according to this criterion; the average d2EGFP signal of these colonies after 35 days (while still under selection pressure) was 425.6, whereas the average d2EGFP signal without selection pressure after 65 days was 290.0. When measured after 105 days, the average signal in the 27 colonies was 300.9. Hence, after an initial decrease, the expression levels in the 27 colonies remained stable according to this criterion (FIG. 8).

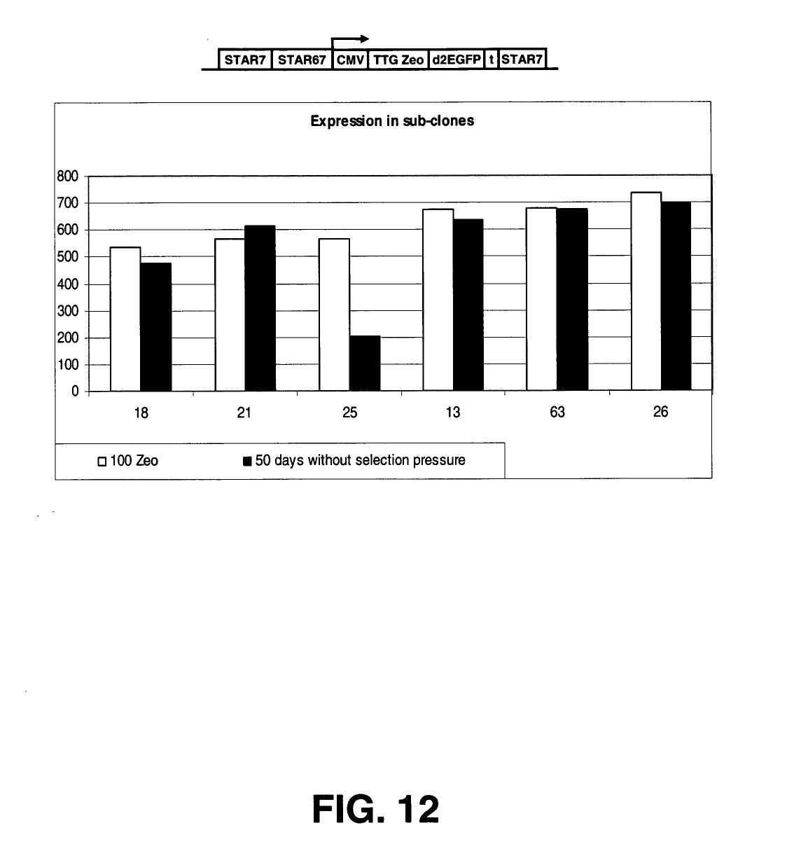

Six of the colonies were subjected to one round of sub-cloning. Cells were sown in 96-wells plates as such that each well contained approximately 0.3 cells. No Zeocin was present in the medium so that from the start the sub clones grew without selection pressure. Of each original colony six sub clones were randomly isolated and grown in 6-wells plates till analysis. In FIG. 12 we compared the original values of the original clones, as already shown in FIG. 4A, with one of the sub clones. In one of the six clones (clone 25), no sub clone was present with d2EGFP signal in the range of the original clone. However, in five out of six cases at least one the sub clones had equal d2EGFP expression levels as the parent clone. These expression levels were determined after 50 days without selection pressure. We conclude that one round of sub cloning is sufficient to obtain a high number of colonies that remain stable for high expression in the absence of selection pressure. This has been confirmed in a similar experiment (not shown).

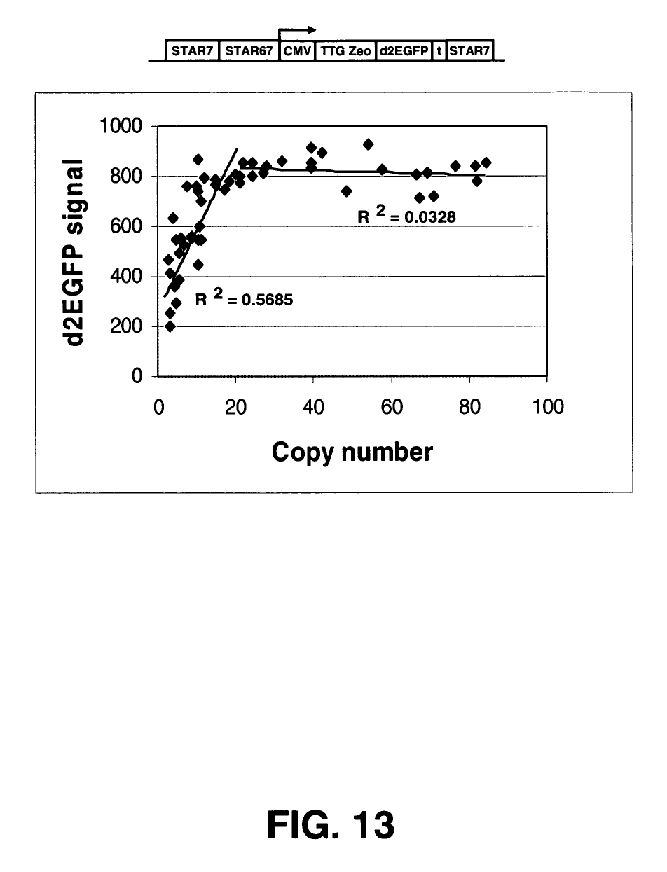

We compared the number of copies that integrated in the TTG Zeo STAR 7/67/7 colonies. DNA was isolated when colonies were 105 days under Zeocin selection pressure (see FIG. 6). As shown in FIG. 13 two populations could be distinguished. In FIG. 13 the cut off was made at 20 copies and the R² value is calculated and shown. Also the R² value from data with higher than 20 copies is shown. In the range from 100 to 800 d2EGFP signal there was a high degree of copy number dependency, as signified by a relatively high R² of 0.5685 (FIG. 13). However, in the population of colonies that fluctuate around a d2EGFP signal of 800 a high variation in copy number was observed (FIG. 13), as signified with a low R² of 0.0328. Together the data show that in the novel selection system, in colonies that contain TTG Zeo STAR 7/67/7 constructs there is copy number dependent d2EGFP expression up to ˜20 copies. Also, although copy number dependency is lost when >20 copies are present, still a substantial proportion of the colonies with high (>800) d2EGFP signal have no more than 30 copies (FIG. 13). This combination between high d2EGFP expression and a relatively low copy number (between 10 and 30) may be important for identifying colonies that remain relatively stable without selection pressure. It is an advantage to have clones with relatively low copy numbers (less than about 30, more preferably less than about 20) that give high expression levels, because such clones are believed to be less amenable to genetic instability. The present selection system allows to generate such clones, including from CHO cells.

### Example 6

### Creation and Testing of Zeocin-Blasticidin-EpCAM Bicistronic Constructs with Differential Translation Efficiencies

To test the selection system on the production of an antibody, the anti-EpCAM antibody (see also example 5 of the incorporated '525 application and of WO2006/005718) was taken as example.

Results

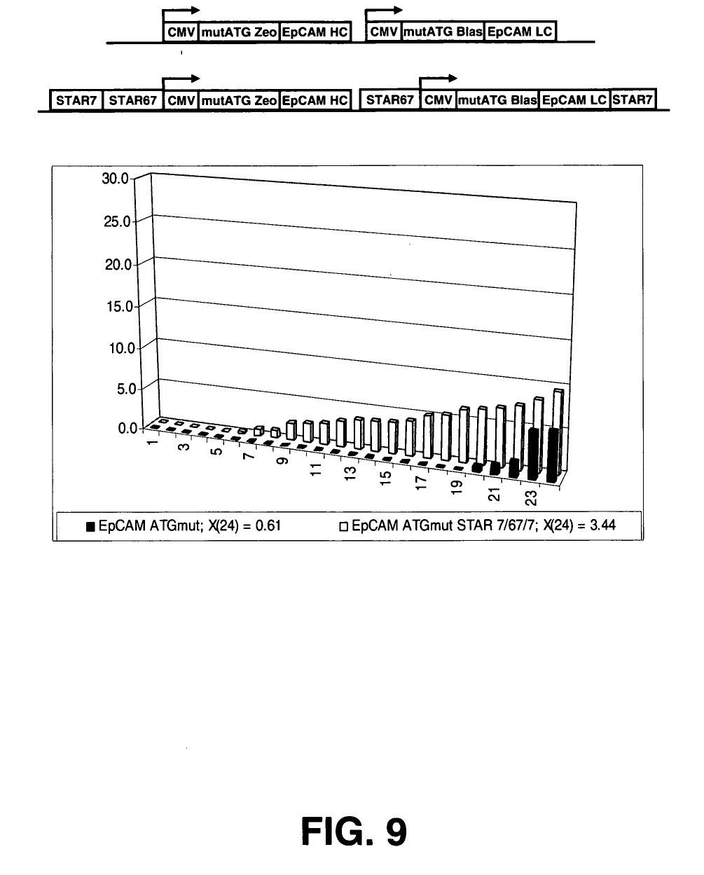

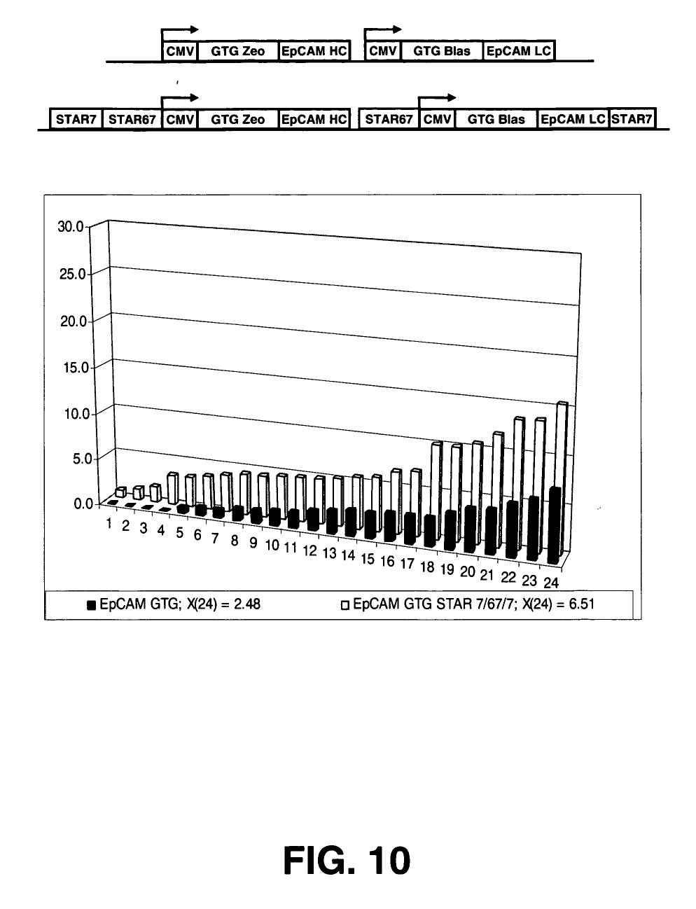

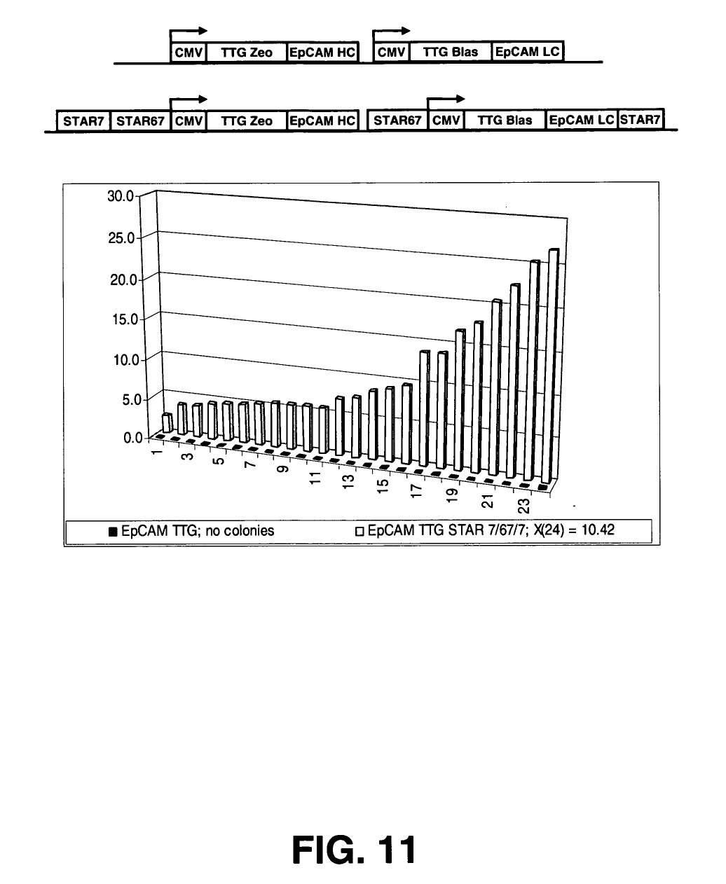

A plasmid was created on which both the heavy chain (HC) and light chain (LC) were placed, each in a separate transcription unit (FIG. 9-11). Expression of both chains was driven by the CMV promoter. Upstream of the EpCAM heavy chain the Zeocin resistance gene was placed, either with the ATGmut/space (FIG. 9), GTG (FIG. 10) or TTG (FIG. 11) as startcodon (see example 2). Upstream of the EpCAM light chain the Blasticidine resistance gene was placed, either with the ATGmut/space (FIG. 9), GTG (FIG. 10) or TTG (FIG. 11) as startcodon (see example 4). Two types of constructs were made, one construct without STAR elements (Control) and one construct with a combination of STAR 7 and 67 elements. The STAR elements were placed as follows: upstream of each CMV promoter (i.e. one for the transcription unit comprising HC and one for the transcription unit comprising LC) STAR 67 was placed and the resulting construct was flanked with a 5′ and 3′ STAR 7 element (FIGS. 9-11). All constructs were transfected to CHO-K1 cells and selected on 100 μg/ml Zeocin and 20 μg/ml Blasticidin (at the same time). After selection independent colonies were isolated and propagated under continuous selection pressure (using 100 μg/ml zeocin and 20 μg/ml blasticidin). FIG. 9 shows that the STAR 7/67/7 combination had a beneficial effect on EpCAM production. The ATGmut/space Zeo and ATGmut/space Blas had no effect on the number of colonies that were formed with plasmids containing STAR elements or not. However, the average EpCAM expression levels of either 24 control versus STAR 7/67/7 colonies ranged from 0.61 pg/cell/day in the control to 3.44 pg/cell/day in the STAR7/67/7 construct (FIG. 9). This is a factor 5.6 increase. Since there were many colonies in the ATGmut/space control with 0 pg/cell/day, also the average EpCAM production in the highest five colonies was compared. In the control ATGmut/space this was 3.0 pg/cell/day, versus 7.8 pg/cell/day with the ATGmut/space STAR 7/67/7 construct, an increase of a factor 2.6.

FIG. 10 also shows that the STAR 7/67/7 combination had a beneficial effect on EpCAM production, using the GTG startcodon for the markers. With the GTG Zeo and GTG Blas STAR 7/67/7 construct approximately 2 times more colonies were formed. Also, the average EpCAM expression levels of either 24 control versus STAR 7/67/7 colonies ranged from 2.44 pg/cell/day in the control to 6.51 pg/cell/day in the STAR7/67/7 construct (FIG. 10). This is a factor 2.7 increase. Also the average EpCAM production in the highest five colonies was compared. In the control GTG this was 5.7 pg/cell/day, versus 13.0 pg/cell/day with the GTG STAR 7/67/7 construct, an increase of a factor 2.3. Also note that the average EpCAM production mediated by the GTG start codon for the selection markers was significantly higher than with the ATGmut/space start codon.

FIG. 11 shows that with the TTG Zeo and TTG Blas control construct no colonies were formed, similar as in example 2. With the STAR 7/67/7 TTG construct colonies were formed. The average EpCAM expression levels of the STAR 7/67/7 TTG colonies was 10.4 pg/cell/day (FIG. 11). This is again higher than with the ATGmut/space and GTG as start codon (see FIGS. 9, 10 for comparison). The average EpCAM production in the highest five TTG STAR 7/67/7 colonies was 22.5 pg/cell/day.

The results show that the selection system can also be applied to two simultaneously produced polypeptides, in this case two polypeptides of a multimeric protein, casu quo an antibody. The EpCAM production closely follows the results obtained with d2EGFP. The TTG as start codon is more stringent than the GTG start codon, which in turn is more stringent than the ATGmut/space (FIGS. 1 and 2). Higher stringency results in a decreasing number of colonies, with no colonies in the case of the TTG control that has no STAR elements, and higher stringency of the selection marker is coupled to higher expression of the protein of interest.

### Example 7

### Creation and Testing of Additional GTG Zeocin-d2EGFP Bicistronic Constructs with Differential Translation Efficiencies

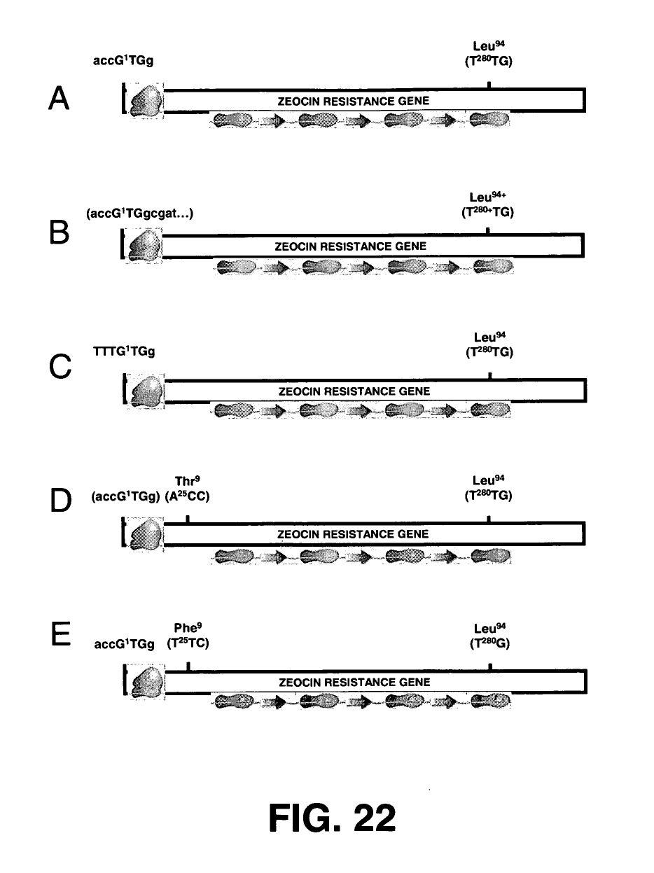

Different versions of the Zeocin resistance gene with mutated startcodons were described in Example 1. Besides the described GTG codons (Example 1, FIG. 22A), additional modified startcodons with distinct translational efficiency are possible. These different Zeocin resistance gene versions were created (FIG. 22) and cloned upstream of the modified d2EGFP gene, as in Example 2.

Materials and Methods

Creation of Plasmids

Four additional GTG constructs were made:

GTG as a start codon in the Zeo resistance gene (FIG. 22A), but followed by a spacer sequence (FIG. 22B). The mutspace-Zeo open reading frame was amplified with primer pair GTGspaceBamHIF (SEQ. ID. NO. 106): GAATTCGGATCCACCGTGGCGATCCAAAGACTGCCAAATCTAG and (wherein the sequence following the underlined sequence comprises the spacer sequence), and ZEOWTreverse (SEQ. ID. NO. 69), the PCR product was cut with EcoRI-BamHI, and ligated into pd2EGFP, cut with EcoRI-BamHI, creating pZEO-GTGspace-d2EGFP.

2) GTG as a start codon in the Zeo resistance gene, but in a bad context (TTTGTG) (FIG. 22C). The Zeo open reading frame was amplified with primer pair ZEOTTTGTGBamHIF (SEQ. ID. NO. 107): GAATTCGGATCCTTTGTGGCCAAGTTGACCAGTGCCGTTCCG and ZEOWTreverse (SEQ. ID. NO. 69), the PCR product was cut with EcoRI-BamHI, and ligated into pd2EGFP, cut with EcoRI-BamHI, creating pZEO(leu)-TTTGTG-d2EGFP.

3) GTG as a start codon in the Zeo resistance gene, instead of ATG (FIG. 22A), but with an additional mutation in the Zeo open reading frame at Pro9, which was replaced with threonine (Thr) (FIG. 22D). The Thr9 mutation was introduced by amplifying the Zeo open reading with primer pair ZEOForwardGTG-Thr9 (SEQ. ID. NO. 108): AATTGGATCCACCGTGGCCAAGTTGACCAGTGCCGTTACCGTGCTC and ZEOWTreverse (SEQ. ID. NO. 69), the PCR product was cut with EcoRI-BamHI, and ligated into pd2EGFP, cut with EcoRI-BamHI, creating pZEO-GTG-Thr9-d2EGFP.

4) GTG as a start codon in the Zeo resistance gene, instead of ATG (FIG. 22A), but with an additional mutation in the Zeo open reading frame at Pro9, with was replaced with Phenylalanine (Phe) (FIG. 22E). The Phe9 mutation was introduced by amplifying the Zeo open reading with primer pair ZEOForward GTG-Phe9 (SEQ. ID. NO. 109): AATTGGATCCACCGTGGCCAAGTTGACCAGTGCCGTTTTCGTGCTC and ZEOWTreverse (SEQ. ID. NO. 69), the PCR product was cut with EcoRI-BamHI, and ligated into pd2EGFP, cut with EcoRI-BamHI, creating pZEO-GTG-Phe9-d2EGFP.

Transfection, Culturing and Analysis of CHO Cells

Transfection, culturing and analysis of CHO-K1 cells was performed as in Example 1.

Results

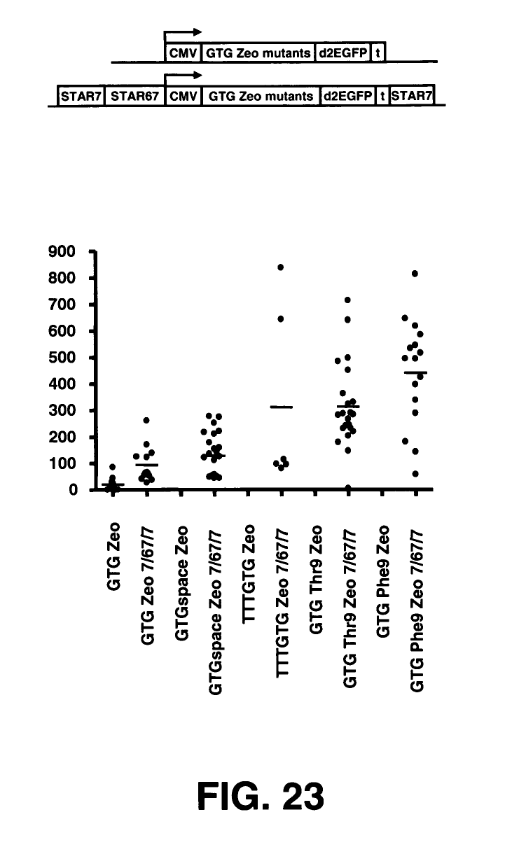

CHO-K1 cells were transfected with constructs that contain the GTG Zeo (FIG. 22A), GTGspace Zeo (FIG. 22B), TTT GTG Zeo (also called: GTGmut Zeo) (FIG. 22C), GTG Thr9 Zeo(leu) (FIG. 22D) and GTG Phe9 Zeo(leu) (FIG. 22D) genes as selection gene, all being cloned upstream of the d2EGFP reporter gene. These five constructs were without STAR elements (Control) or with STAR elements 7 and 67 upstream of the CMV promoter and STAR 7 downstream from the d2EGFP gene (FIG. 22). FIG. 23 shows that of the control constructs without STAR elements only the GTG Zeo construct without STAR elements gave colonies that expressed d2EGFP protein. In contrast, all constructs containing STAR elements gave colonies that expressed d2EGFP protein. The mean d2EGFP fluorescence signal of 11 GTG Zeo Control colonies was 20.3, of 13 GTG Zeo colonies with STARs 7/67/7 104.9, of 24 GTG space Zeo 7/67/7 colonies 201.5, of 6 TTT GTG Zeo 7/67/7 colonies 310.5, of 22 GTG Thr9 Zeo 7/67/7 colonies 423, and of 16 GTG Phe9 Zeo colonies 550.2 (FIG. 23).

The higher stringencies of the novel GTG mutations correlate with higher mean fluorescence signals (FIG. 23). The TTT GTG Zeo 7/67/7, however, gave only two high expressing colonies and a few low expressing colonies. This may indicate that this mutation is at the brink of the stringency that these cells can bear with a fixed concentration of Zeocin added to the culture medium.

The Thr9 and Phe9 mutations do not influence the translation efficiency of the Zeo mutants. Instead they reduce the functionality of the Zeocin resistance protein, by preventing an optimal interaction between the two halves of the Zeocin resistance protein (Dumas et al, 1994). This implies that more of the protein has to be produced to achieve resistance against the Zeocin in the culture medium. As a consequence, the entire cassette has to be transcribed at a higher level, eventually resulting in a higher d2EGFP expression level.

It is concluded that the use of the described translation efficiencies of the Zeocin resistance mRNA result in higher expression levels of the d2EGFP protein, this in combination with STAR elements.

This example further demonstrates the possibility to provide for fine-tuning of the stringency of the selection system of the invention, to achieve optimal expression levels of a protein of interest. Clearly, the person skilled in the art will be capable of combining these and other possibilities within the concepts disclosed herein (e.g. mutate the zeocin at position 9 to other amino acids, or mutate it in other positions; use a GTG or other startcodon in a non-optimal translation initition context for zeocin or other selection markers; or mutate other selection markers to reduce their functionality, for instance use a sequence coding for a neomycin resistance gene having a mutation at amino acid residue 182 or 261 or both, see e.g. WO 01/32901), and the like, to provide for such fine-tuning, and by simply testing determine a suitable combination of features for the selection marker, leading to enhanced expression of the polypeptide of interest.

### Example 8

### Creation and Testing of Additional TTG Zeocin-d2EGFP Bicistronic Constructs with Differential Translation Efficiencies

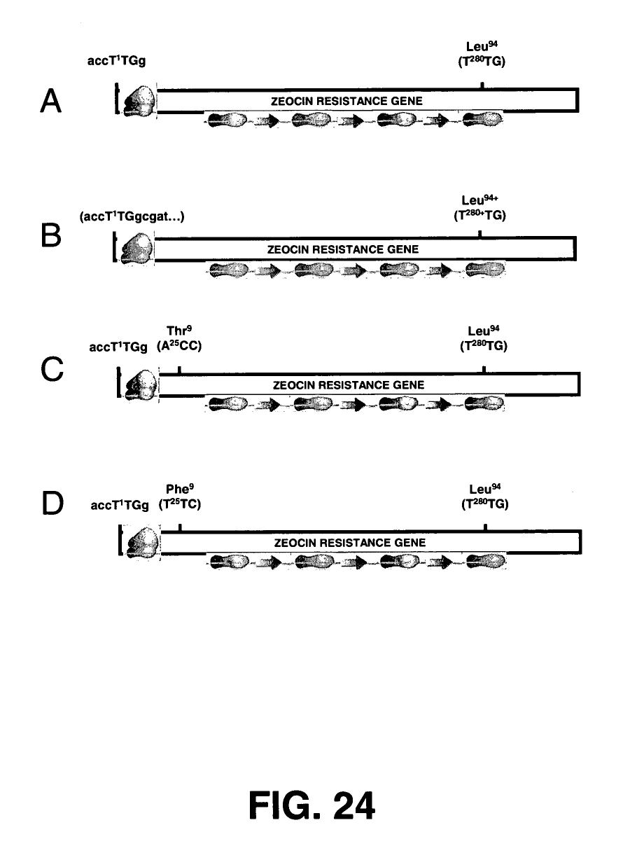

Different versions of the Zeocin resistance gene with mutated startcodons were described in Example 1. Besides the described TTG codons (FIG. 24A) additional modified startcodons with distinct translational efficiency are possible. These different Zeocin resistance gene versions were created and cloned upstream of the modified d2EGFP gene (FIG. 24).

Materials and Methods

Creation of Plasmids

Three additional TTG constructs were made:

1) TTG as a start codon in the Zeo resistance gene (FIG. 24A), but followed by a spacer sequence (FIG. 24B). The Zeo open reading frame (with the spacer sequence) was amplified with primer pair TTGspaceBamHIF (SEQ. ID. NO. 110): GAATTCGGATCCACCTTGGCGATCCAAAGACTGCCAAATCTAG and ZEOWTreverse(SEQ. ID. NO. 69), the PCR product was cut with EcoRI-BamHI, and ligated into pd2EGFP, cut with EcoRI-BamHI, creating pZEO-TTGspace-d2EGFP.

2) TTG as a start codon in the Zeo resistance gene, instead of ATG (FIG. 24A), but with an additional mutation in the Zeo open reading frame at Pro9, with was replaced with threonine (Thr) (FIG. 24C). The Thr9 mutation was introduced by amplifying the Zeo open reading with primer pair ZEOForwardTTG-Thr9 (SEQ. ID. NO. 111): AATTGGATCCACCTTGGCCAAGTTGACCAGTGCCGTTACCGTGCTC and ZEOWTreverse (SEQ. ID. NO. 69), the PCR product was cut with EcoRI-BamHI, and ligated into pd2EGFP, cut with EcoRI-BamHI, creating pZEO-TTG-Thr9-d2EGFP.

3) TTG as a start codon in the Zeo resistance gene, instead of ATG (FIG. 24A), but with an additional mutation in the Zeo open reading frame at Pro9, with was replaced with Phenylalanine (Phe) (FIG. 24D). The Phe9 mutation was introduced by amplifying the Zeo open reading with primer pair ZEOForwardTTG-Phe9 (SEQ. ID. NO. 112): AATTGGATCCACCTTGGCCAAGTTGACCAGTGCCGTTTTCGTGCTC and ZEOWTreverse (SEQ. ID. NO. 69), the PCR product was cut with EcoRI-BamHI, and ligated into pd2EGFP, cut with EcoRI-BamHI, creating pZEO-TTG-Phe9-d2EGFP.

Results

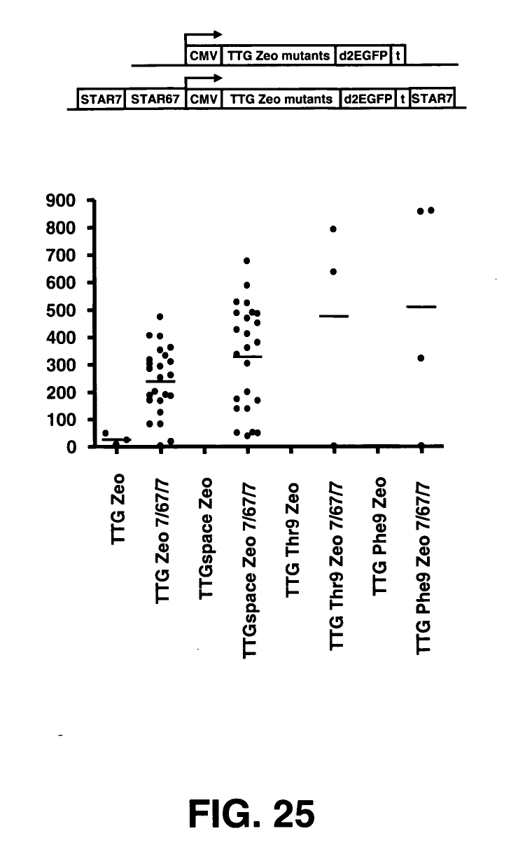

CHO-K1 cells were transfected with constructs that contain the TTG Zeo (FIG. 24A), TTGspace Zeo (FIG. 24B), TTG Thr9 Zeo (FIG. 24C) and TTG Phe9 Zeo (FIG. 24D) genes as selection gene, all being cloned upstream of the d2EGFP reporter gene. These four constructs were without STAR elements (Control) or with STAR elements 7 and 67 upstream of the CMV promoter and STAR 7 downstream from the d2EGFP gene (FIG. 24). FIG. 25 shows that of the control constructs without STAR elements only the TTG Zeo construct without STAR elements gave colonies that expressed d2EGFP protein. In contrast, all constructs containing STAR elements gave colonies that expressed d2EGFP protein. The mean d2EGFP fluorescence signal of 3 TTG Zeo Control colonies was 26.8, of 24 TTG Zeo colonies with STARs 7/67/7 426.8, of 24 TTGspace Zeo 7/67/7 colonies 595.7, of 2 TTG Thr9 Zeo 7/67/7 colonies 712.1, and of 3 TTG Phe9 Zeo colonies 677.1 (FIG. 25).

The higher stringencies of the novel TTG mutations correlate with higher mean fluorescence signals (FIG. 25). The TTG Thr9 Zeo 7/67/7 and TTG Phe9 Zeo 7/67/7 constructs, however, gave only two high expressing colonies each and a few low expressing colonies. This may indicate that these mutations are at the brink of the stringency that the cells can bear with a fixed concentration of Zeocin added to the culture medium.

It is concluded that the use of the described translation efficiencies of the Zeocin resistance mRNA result in higher expression levels of the d2EGFP protein, this in combination with STAR elements.

### Example 9

### Creation and Testing of Puromycin-d2EGFP Bicistronic Constructs with Differential Translation Efficiencies

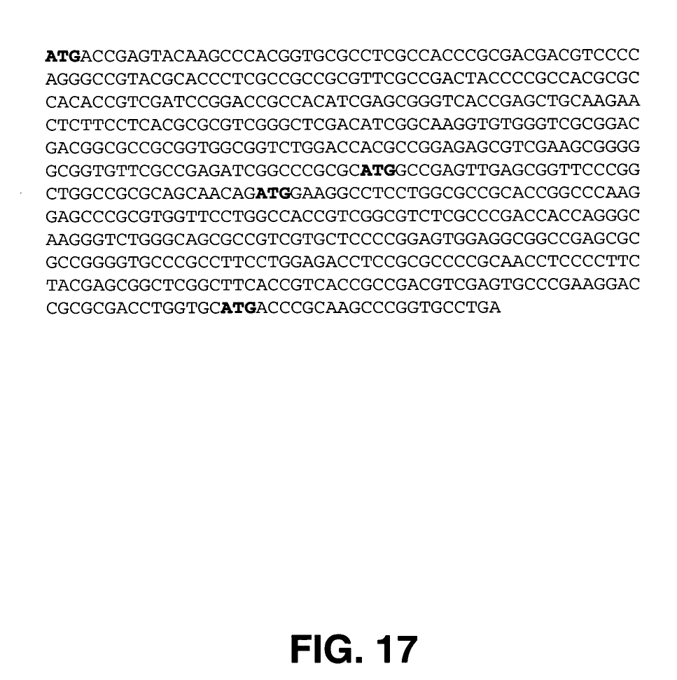

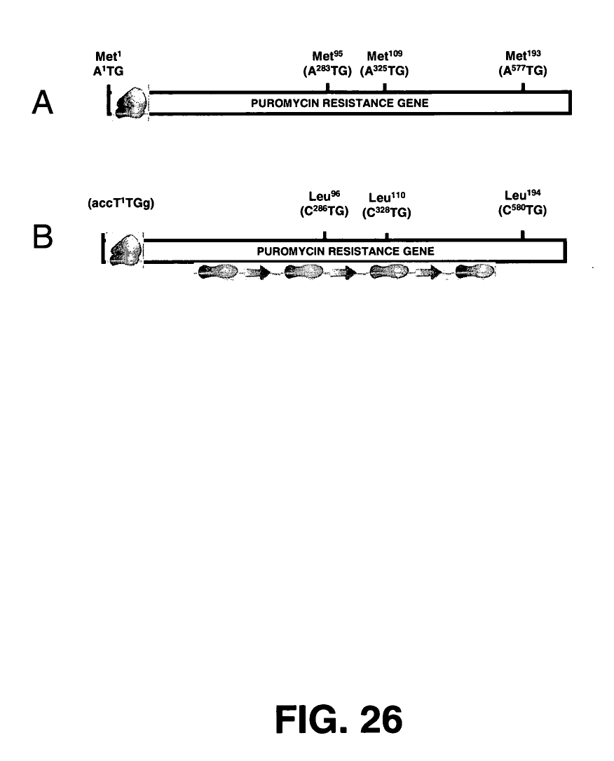

There are three internal ATGs in the puromycin resistance gene, each of which codes for a methionine (FIG. 17, FIG. 26A). These ATGs have to be eliminated (FIG. 26B,C), since they will serve as start codon when the ATG startcodon (or the context thereof) has been modified, and this will result in peptides that do not resemble puromycin resistance protein. More importantly, these ATGs will prevent efficient translation of the gene of interest, as represented by d2EGFP in this example for purposes of illustration. The methionines were changed into leucine, like in the zeocin resistance protein (example 1). However, instead of using the TTG codon for leucine (for instance in Zeocin in example 1), now the CTG codon for leucine was chosen (in humans, for leucine the CTG codon is used more often than the TTG codon). To eliminate the internal ATGs, the puromycin resistance protein open reading frame was first amplified with 4 primer pairs, generating 4 puromycin resistance protein fragments. The primer pairs were:

| | |
|---|---|
| PURO BamHI F: | |
| GATCGGATCCATGGTTACCGAGTACAAGCCCA | (SEQ. ID. NO. 113) |
| CGGT, | |
| PURO300 R LEU: | |
| CAGCCGGGAACCGCTCAACTCGGCCAGGCGCG | (SEQ. ID. NO. 114) |
| GGC; | |
| and | |
| PURO300FLEU: | |
| CGAGTTGAGCGGTTCCCGGCTGGCCGCGCAGC | (SEQ. ID. NO. 115) |
| AACAGCTGGAAGGCCTC, | |
| PURO600RLEU: | |
| AAGCTTGAATTCAGGCACCGGGCTTGCGGGTC | (SEQ. ID. NO. 116) |
| AGGCACCAGGTC. | |

This generates two PCR products, corresponding to the 5′ and 3′ part of the puromycin resistance gene. The two products were added together and amplified with PURO BamHI F (SEQ. ID. NO. 113)—PURO600RLEU (SEQ. ID. NO. 1116). The resulting PCR product was cut with BamHI-EcoRI and ligated, creating pCMV-ATGPURO (leu). Sequencing of this clone verified that all three internal ATGs had been converted. The entire puromycin open reading frame was then amplified with PUROBamHI TTG1F (SEQ. ID. NO. 117): GAATTCGGATCCACCTTGGTTACCGAGTACAAGCCCACGGTG and PURO600RLEU (SEQ. ID. NO. 116). This primer introduces an extra codon (GTT) directly after the TTG startcodon, because the ‘G’ at nucleotide +4 is introduced for an optimal context, and hence two more nucleotides are introduced to preserve the reading frame.
Results

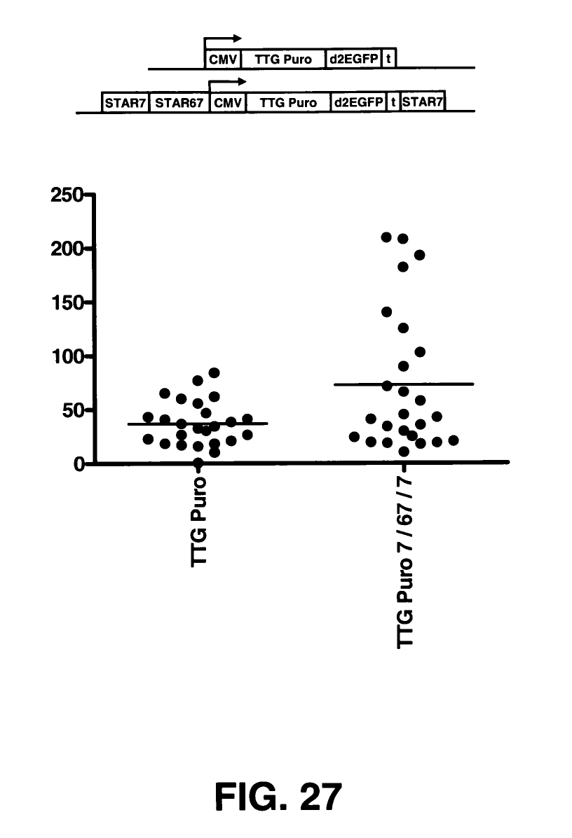

CHO-K1 cells were transfected with the construct that contains the TTG Puro (FIG. 27) gene as selection gene, cloned upstream of the d2EGFP reporter gene. Selection was under 10 μg/ml puromycin. The construct was without STAR elements (Control) or with STAR elements 7 and 67 upstream of the CMV promoter and STAR 7 downstream from the d2EGFP gene (FIG. 27). FIG. 27 shows that the average d2EGFP fluorescence signal of 24 TTG Puro Control colonies was 37.9, of 24 TTG Puro colonies with STARs 7/67/7 75.5. Moreover, when the average of the five highest values is taken, the d2EGFP fluorescence signal of TTG Puro Control colonies was 69.5, and of TTG Puro colonies with STARs 7/67/7 186.1, an almost three-fold increase in d2EGFP fluorescence signal. This shows that the described, modified translation efficiency of the Puromycin resistance mRNA result in higher expression levels of the d2EGFP protein, this in combination with STAR elements.

This experiment demonstrates that the puromycin resistance gene can be mutated to remove the ATG sequences therefrom, while remaining functional. Moreover it is concluded that the selection method of the invention also works with yet another selection marker, puromycin.

### Example 10

### Creation and Testing of Neomycin Constructs with Differential Translation Efficiencies

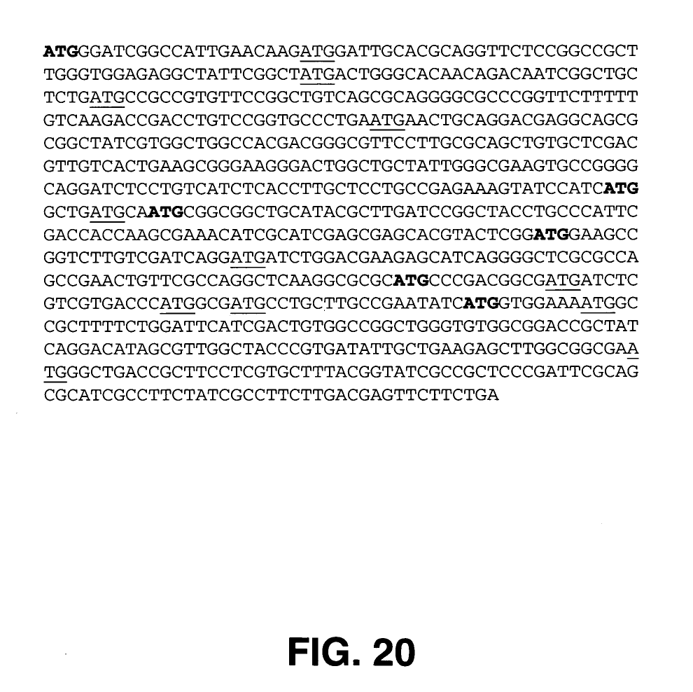

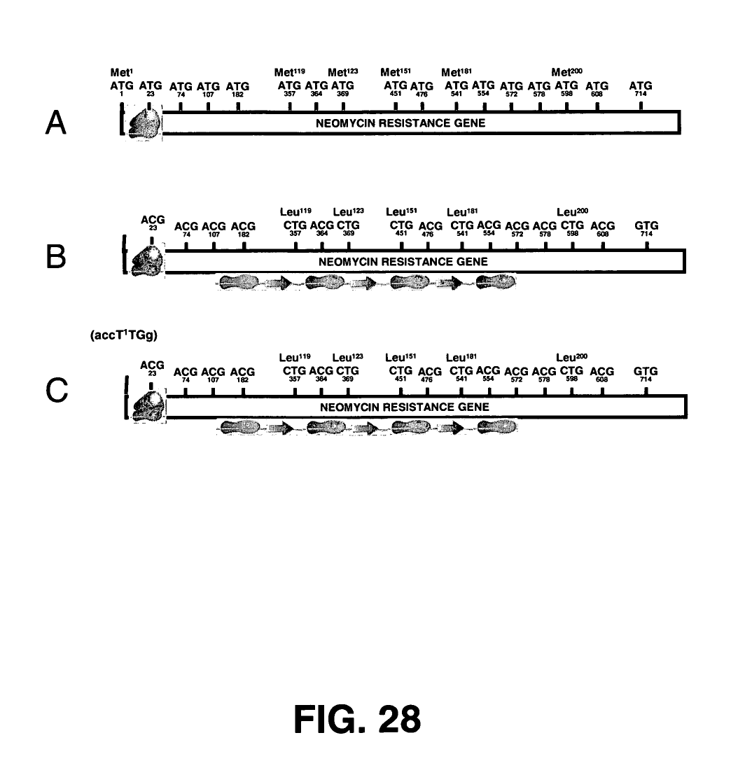

There are sixteen internal ATGs in the neomycin resistance gene, five of which code for a methionine in the neomycin open reading frame (FIG. 20, FIG. 28A). All these sixteen ATGs have to be eliminated (FIG. 28B,C), since they will serve as start codon when the ATG startcodon (or the context thereof) has been modified, and this will result in peptides that do not resemble neomycin resistance protein, and this will decrease the translation from the downstream open reading frame coding for the polypeptide of interest in the transcription units of the invention. To eliminate the internal ATGs, the neomycin resistance protein open reading frame was entirely synthesized by a commercial provider (GeneArt, Germany), wherein all internal coding ATGs (for Met) where replaced by CTGs (coding for Leu), and non-coding ATGs were replaced such that a degenerated codon was used and hence no mutations in the protein sequence resulted; the synthesised sequence of the neomycin is given in SEQ. ID. NO. 118. In order to replace the ATG start codon with GTG (FIG. 28B) or TTG FIG. 28C), the synthesized neomycin gene was amplified with primer pairs NEO-F-HindIII (SEQ. ID. NO. 120): GATCAAGCTTTTGGATCGGCCATTGAAACAAGACGGATTG and NEO EcoRI 800R (SEQ. ID. NO. 121): AAGCTTGAATTCTCAGAAGAACTCGTCAAGAAGGCG.

Results

E. coli bacteria were used to test the functionality of the neomycin resistance protein from which all ATGs were removed. E. coli bacteria were transformed with the constructs that contain the GTG Neo (FIG. 28B) or TTG Neo (FIG. 28C) gene as selection gene. Selection took place by growing the bacteria on kanamycin. Only a functional neomycin resistance gene can give resistance against kanamycin. Transformation with either modified Neo gene resulted in the formation of E. coli colonies, from which the plasmid containing the gene could be isolated. This shows that the described, modified translation efficiencies of the Neomycin resistance mRNAs, as well as the removal of all ATGs from the Neo open reading frame result in the production of functional neomycin resistance protein.

The mutated neomycin resistance genes are incorporated in a multicistronic transcription unit of the invention, and used for selection with G418 or neomycin in eukaryotic host cells.

### Example 11

### Creation and Testing of dhfr Constructs with Differential Translation Efficiencies

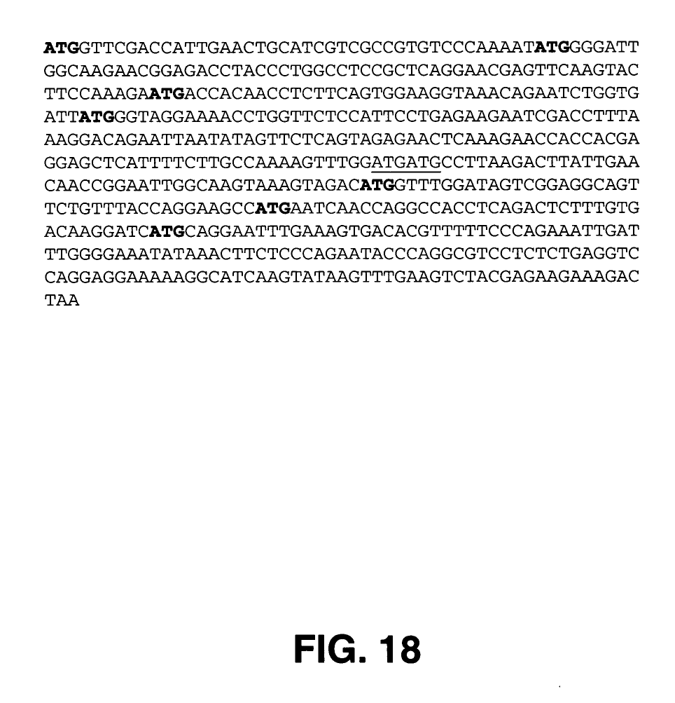

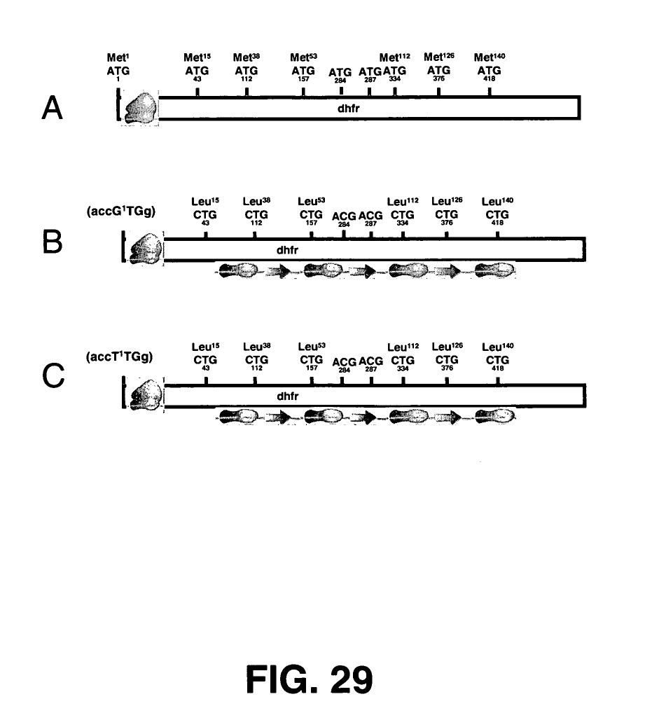

There are eight internal ATGs in the dhfr gene, six of which code for a methionine in the dhfr open reading frame (FIG. 18, FIG. 29A). All these ATGs have to be eliminated (FIGS. 29B,C), since they will serve as start codon when the ATG startcodon (or the context thereof) has been modified, and this will result in peptides that do not resemble dhfr protein, and will decrease the translation from the downstream open reading frame coding for the polypeptide of interest in the transcription units of the invention. To eliminate the internal ATGs, the dhfr protein open reading frame was entirely synthesized (SEQ. ID. NO. 122), as described above for neomycin. In order to replace the ATG start codon with GTG (FIG. 29B) or TTG (FIG. 29C), the synthesized DHFR gene was amplified with primers DHFR-F-HindIII (SEQ. ID. NO. 124): GATCAAGCTTTTGTTCGACCATTGAACTGCATCGTC and DHFR-EcoRI-600-R (SEQ. ID. NO. 125): AGCTTGAATTCTTAGTCTTTCTTCTCGTAGACTTC.

Results

E. coli bacteria were used to test the functionality of the dhfr protein from which all ATGs were removed. E. coli was transformed with the constructs that contain the GTG dhfr (FIG. 29B) or TTG dhfr (FIG. 29C) gene. Selection took place by growing the bateria on trimethoprim (Sigma T7883-56). Only a functional dhfr gene can give resistance against trimethoprim. Transformation with either modified dhfr gene resulted in the formation of E. coli colonies, from which the plasmid containing the gene could be isolated. This shows that the described, modified translation efficiencies of the dhfr mRNAs, as well as the removal of all ATGs from the dhfr open reading frame result in the production of functional dhfr protein.

The mutated dhfr genes are incorporated in a multicistronic transcription unit of the invention, and used for selection with methotrexate in eukaryotic host cells.

### Example 12

### Testing of Zeocin- and Blasticidin Constructs with Differential Translation Efficiencies in PER.C6 Cells

Various Zeocin and blasticidin genes with mutated startcodons, all cloned upstream of the d2EGFP gene were tested in the PER.C6 cell line.

Results

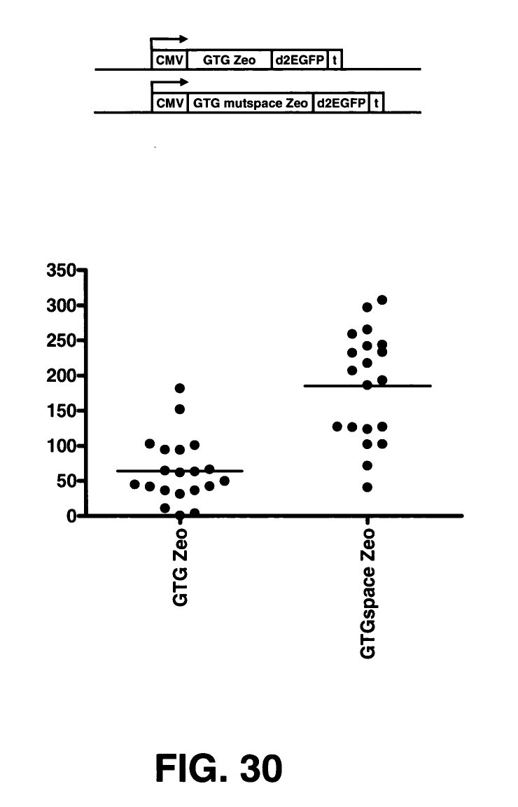

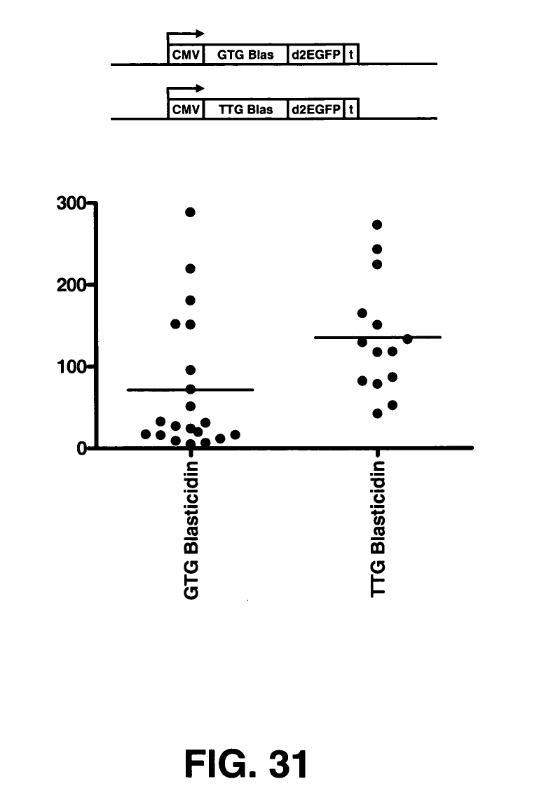

The GTG Zeocin and GTGspace Zeocin resistance gene modifications (see also Example 7; FIG. 30) and the GTG blasticidin and TTG blasticidin resistance gene modifications (see also Example 4; FIG. 31), all cloned upstream of the d2EGFP gene were transfected to PER.C6 cells. As shown in FIG. 30, transfection with both the GTG Zeocin and GTGspace Zeocin gene resulted in colonies that expressed d2EGFP. The average d2EGFP fluorescence signal of 20 GTG Zeo colonies was 63.8, while the average d2EGFP signal of 20 GTGspace Zeo colonies was 185, demonstrating that also in PER.C6 cells the GTGspace Zeo has a higher translation stringency than the GTG Zeo mRNA.

As shown in FIG. 31, transfection with both the GTG Blasticidin and TTG Blasticidin gene resulted in colonies that expressed d2EGFP. The average d2EGFP fluorescence signal of 20 GTG Blasticidin colonies was 71.4, while the average d2EGFP fluorescence signal of 20 TTG Blasticidin colonies was 135, demonstrating that also in PER.C6 cells the TTG Blasticidin has a higher translation stringency than the GTG Blasticidin mRNA.

This example demonstrates that the selection system of the invention can also be used in other cells than CHO cells.

### Example 13

### Testing of the Addition of a Transcriptional Pause Signal to a TTG Zeocin-d2EGFP Construct

A TRAnscription Pause (TRAP) sequence is thought to, at least in part, prevent formation of antisense RNA or, to at least in part, prevent transcription to enter said protein expression unit (see WO 2004/055215). A TRAP sequence is functionally defined as a sequence which when placed into a transcription unit, results in a reduced level of transcription in the nucleic acid present on the 3′ side of the TRAP when compared to the level of transcription observed in the nucleic acid on the 5′ side of the TRAP, and non-limiting examples of TRAP sequences are transcription termination signals. In order to function to prevent or decrease transcription to enter the transcription unit, the TRAP is to be placed upstream of a promoter driving expression of the transcription unit and the TRAP should be in a 5′ to 3′ direction. In order to prevent at least in part formation of antisense RNA, the TRAP should be located downstream of the open reading frame in a transcription unit and present in a 3′ to 5′ direction (that is, in an opposite orientation as the normal orientation of a transcriptional termination sequence that is usually present behind the open reading frame in a transcription unit). A combination of a TRAP upstream of the promoter in a 5′ to 3′ orientation and a TRAP downstream of the open reading frame in a 3′ to 5′ oreintation is preferred. Adding a TRAP sequence to a STAR element improves the effects of STAR elements on transgene expression (see WO 2004/055215). Here we test the effects of the TRAP sequence in the context of the TTG Zeo resistance gene.

Results

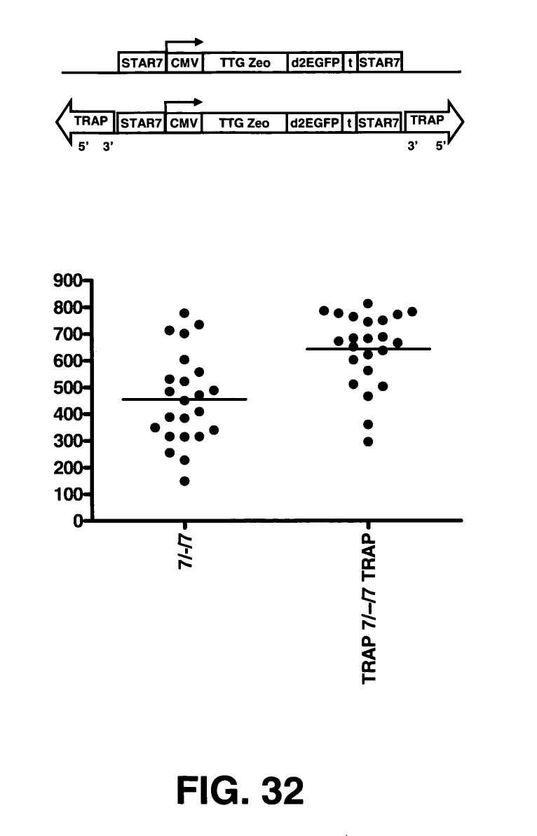

The TTG Zeocin-d2EGFP cassette that was flanked with STAR7 elements (FIG. 32) was modified by the addition of the SPA/pause TRAP sequence (see WO 2004/055215); SEQ. ID. NO. 126), both upstream of the 5′ STAR7 (in 5′ to 3′ direction) and downstream of the 3′ STAR7 (in 3′ to 5′ direction) (FIG. 32). Both STAR 7/7 and TRAP-STAR 7/7-TRAP containing vectors were transfected to CHO-K1. Stable colonies were isolated and the d2EGFP fluorescence intensities were measured. As shown in FIG. 43 the average d2EGFP fluorescence signal of 23 TTG Zeo STAR 7/7 colonies was 455.1, while the average d2EGFP fluorescence signal of 23 TTG Zeo TRAP-STAR 7/7-TRAP colonies was 642.3. The average d2EGFP fluorescence signal in highest 5 TTG Zeo STAR 7/7 colonies was 705.1, while the average d2EGFP fluorescence signal of 5 TTG Zeo TRAP-STAR 7/7-TRAP colonies was 784.7.

This result indicates that the addition of TRAPs does not enhance the d2EGFP fluorescence signal in the highest colonies, but that there is a significant raise in the number of high expressing colonies. Whereas only 5 TTG Zeo STAR 7/7 colonies had d2EGFP signal above 600, 17 TTG Zeo TRAP-STAR 7/7-TRAP colonies had a d2EGFP fluorescence signal above 600.

In the experiment 3 μg DNA of each plasmid was transfected. However, whereas the transfection efficiency was similar, the total number of colonies with the TTG Zeo STAR 7/7 plasmid was 62, while the total number of colonies with the TTG Zeo TRAP-STAR 7/7-TRAP plasmid was 116, almost a doubling.

We conclude that addition of TRAP elements to the STAR containing plasmids with modified Zeocin resistance gene translation codons results in a significantly higher overall number of colonies and that more colonies are present with the highest expression levels.

### Example 14

### Copy-Number Dependency of Expression

We analyzed the EpCAM antibody expression levels in relation to the number of integrated EpCAM DNA copies.

Results

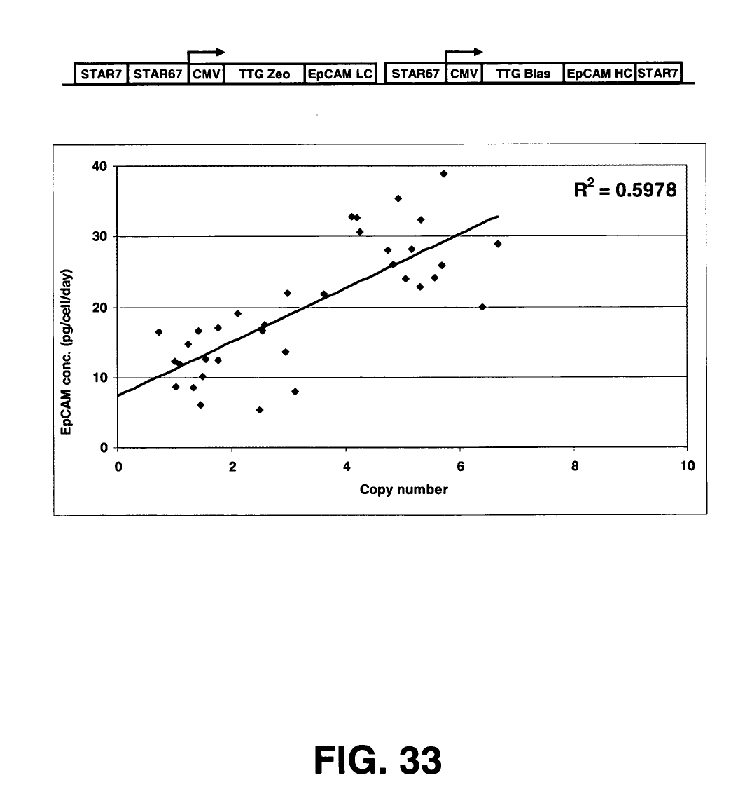

The construct that was tested was TTG-Zeo-Light Chain (LC)-TTG-Blas-Heavy Chain (HC), both expression units being under the control of the CMV promoter (see FIG. 33). This construct contained STAR 7 and 67 (see FIG. 33). Selection conditions were such that with 200 μg/ml Zeocin and 20 pg/ml Blasticidin in the culture medium no control colonies (no STARs) survived and only STAR 7/67/7 colonies survived.

DNA was isolated when colonies were 60 days under Zeocin and Blasticidin selection pressure (see FIG. 33). The R² value is calculated and shown. In the entire range from 5 to 40 pg/cell/day EpCAM there was a high degree of copy number dependency, as signified by a relatively high R² of 0.5978 (FIG. 33). The data show that in the novel selection system, in colonies that contain TTG Zeo-TTG Blas EpCAM STAR 7/67/7 constructs there is copy number dependent EpCAM expression.

### Example 15

### Methotrexate Induction of Higher EpCAM Expression

We analyzed EpCAM antibody expression levels after incubation of clones with methotrexate (MTX). The purpose of this experiment was to determine whether amplification of a STAR-containing construct would result in higher EpCAM expression. MTX acts through inhibition of the dhfr gene product. While some CHO strains that are dhfr-deficient have been described, CHO-K1 is dhfr⁺. Therefore relatively high concentrations of MTX in the culture medium have to be present to select for amplification by increased MTX concentrations in CHO-K1 cells.

Results

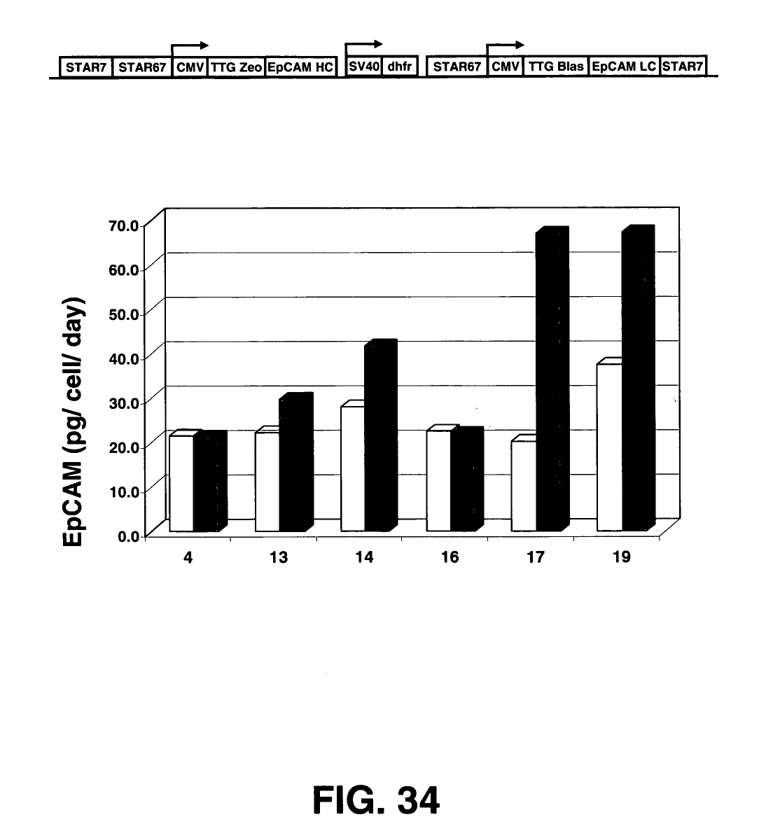

The construct that was tested was TTG-Zeo-Heavy Chain (HC)-TTG-Blas-Light Chain (LC), both expression units being under the control of the CMV promoter. Upstream of each CMV promoter STAR67 was positioned and STAR7 was used to flank the entire cassette (see also Example 6, FIG. 11 for such a construct). This construct was further modified by placing an SV40-dhfr cassette (a mouse dhfr gene under control of an SV40 promoter) between the HC and LC cassettes, upstream of the second STAR67 (FIG. 34). CHO-K1 cells were transfected. Selection was done with 100 μg/ml Zeocin and 10 μg/ml Blasticidin in the culture medium. No control colonies (without STAR elements) survived and only colonies with constructs containing the STAR elements survived. Colonies were isolated and propagated before measuring EpCAM expression levels. Six colonies that produced between 20 and 35 pg/cell/day were transferred to medium containing 100 nM MTX. This concentration was raised to 500 nM, 1000 nM and finally to 2000 nM with two weeks periods in between each step. After two weeks on 2000 nM MTX, EpCAM concentrations were measured. As shown in FIG. 34, four colonies showed enhanced EpCAM production. Colony 13: from 22 to 30; colony 14: from 28 to 42; colony 17: from 20 to 67 and colony 19: from 37 to 67 pg/cell/day. Colonies 4 and 16 showed no enhanced EpCAM expression. We conclude that addition of methotrexate to the culture medium of CHO-K1 colonies created with the selection system of the invention can result in enhanced protein expression. Hence, STAR elements and the selection method of the invention can be combined with and are compatible with MTX-induced enhancement of protein expression levels.

### Example 16

### TTG-Zeo Selection Operates in the Context of Different Promoters

We analyzed d2EGFP expression levels in the context of the TTG Zeo selection marker and different promoters. We compared the action of STAR elements in the context of the CMV enhancer/promoter, the SV40 enhancer/promoter and the CMV enhancer/β-actin promoter.

Results

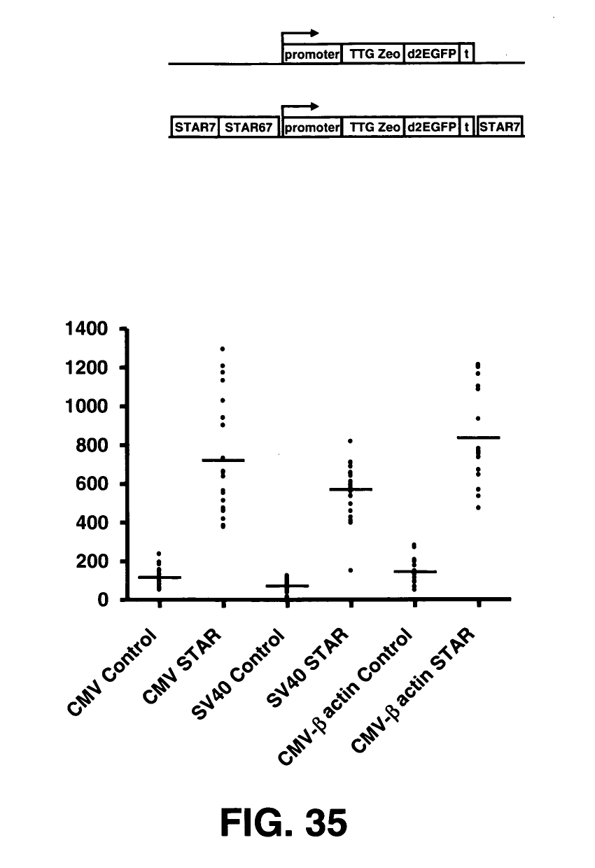

In FIG. 35 we indicate the promoters we tested in the context of the TTG Zeo selection marker. The tested plasmids consisted of the indicated control constructs with three different promoters and STAR constructs which were flanked with STAR 7 and STAR 67 at the 5′ end and STAR 7 at the 3′ end. The constructs were transfected to CHO-K1 cells and selection was performed with 200 μg/ml Zeocin in the culture medium. Up to 23 independent colonies were isolated and propagated before analysis of d2EGFP expression levels. As shown in FIG. 35, incorporation of STAR elements in constructs with the CMV enhancer/promoter, the SV40 enhancer/promoter or the CMV enhancer/β-actin promoter all resulted in the formation of colonies with higher d2EGFP expression levels than with the corresponding control constructs. This shows that the selection system of the invention, in combination with STAR elements, operates well in the context of different promoters. Further analysis showed that the mean of CMV-driven d2EGFP values was significantly higher than the mean of SV40-driven d2EGFP values (p<0.05). In contrast, the mean of CMV-driven d2EGFP values did not significantly differ from CMV/β actin-driven d2EGFP values (p=0.2).

### Example 17

### Comparison of Different STAR Elements in the TTG-Zeo Selection System

We analyzed d2EGFP expression levels in the context of the CMV promoter-TTG Zeo selection marker and 53 different STAR elements, to obtain more insight in which STAR elements give the best results in this context.

Results

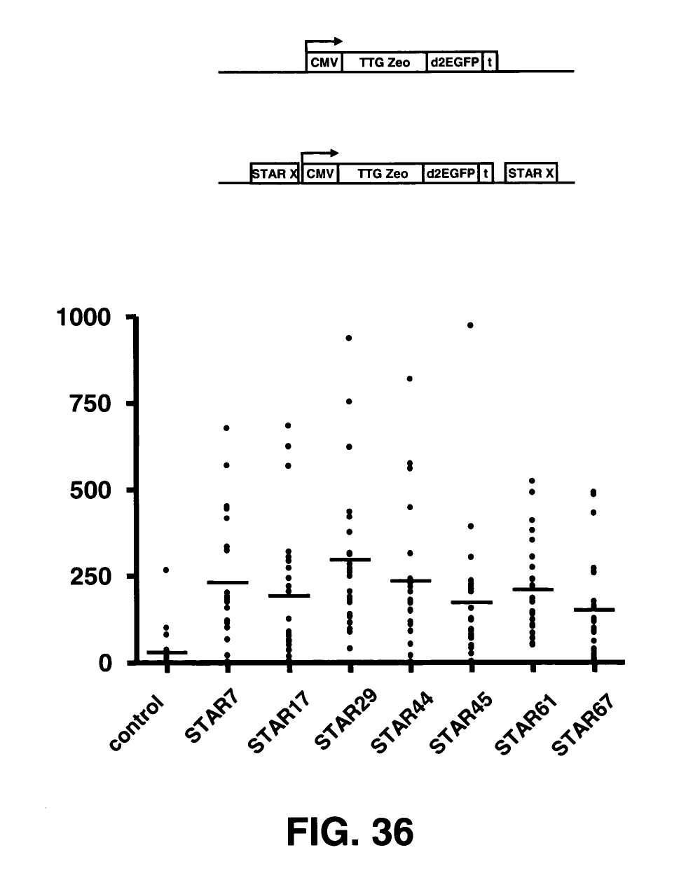

We cloned 53 STAR elements up-and downstream of the CMV promoter-TTG Zeo-d2EGFP cassette. The following STAR elements were tested in such constructs: STAR2-12, 14, 15, 17-20, 26-34, 36, 37, 39, 40, 42-49, 51, 52, 54, 55, 57-62, 64, 65, 67. The constructs were transfected to CHO-K1 cells and selection was performed with 200 μg/ml Zeocin in the culture medium. Up to 24 independent colonies were isolated and propagated before analysis of d2EGFP expression levels. Incorporation of STAR elements in the constructs resulted in different degrees of enhanced d2EGFP expression, as compared to the control. Incorporation of STAR elements 14, 18 and 55 in this experiment did not result in an increase of average d2EGFP expression over the control (no STAR element). Although some constructs (with STAR elements 2, 3, 10, 42, 48 and 49) in this experiment gave rise to only a few colonies, all tested STAR elements except 14, 18 and 55 resulted in average d2EGFP expression levels higher than for the control. It should be noted that some STAR elements may act in a more cell type specific manner and that it is well possible that STAR 14, 18 and 55 work better in other cell types, with other promoters, other selection markers, or in different context or configuration than in the particular set of conditions tested here. Addition of 10 STAR elements, namely STAR elements 7, 9, 17, 27, 29, 43, 44, 45, 47 and 61, induced average d2EGFP expression levels higher than 5 times the average d2EGFP expression level of the control. We retransformed the control and 7 constructs with STAR elements and repeated the experiment. The results are shown in FIG. 36. Incorporation of STAR elements in the constructs resulted in different degrees of enhanced d2EGFP expression, as compared to the control (FIG. 47). The average d2EGFP expression level in colonies transfected with the control construct was 29. The averages from d2EGFP expression levels in colonies with the 7 different STAR constructs ranged between 151 (STAR 67) and 297 (STAR 29). This is a factor of 5 to 10-fold higher than the average in the control colonies.

We conclude that a) the vast majority of STAR elements have a positive effect on gene expression levels, b) there is variation in the degree of positive effects induced by the different STAR elements, and c) 10 out of 53 tested STAR elements induce more than 5-fold average d2EGFP expression levels, as compared to the control, and that STAR elements can induce a 10-fold higher average d2EGFP expression level, as compared to the control.

### Example 18

### Other Chromatin Control Elements in the Context of a Selection System of the Invention

DNA elements such as the HS4 hypersensitive site in the locus control region of the chicken β-globin locus (Chung et al, 1997), matrix attachment regions (MAR) (Stief et al, 1989) and a ubiquitous chromatin opening element (UCOE) (Williams et al, 2005) have been reported to have beneficial effects on gene expression when these DNA elements are incorporated in a vector. We combined these DNA elements with the selection system of the invention.

Results

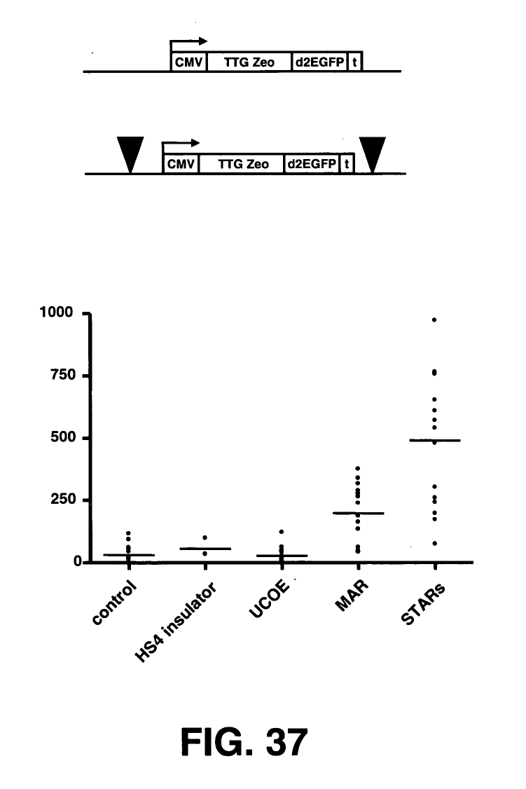

The 1.25 kb HS4 element was cloned into the cassette encompassing the CMV promoter, TTG Zeo and d2EGFP by a three way ligation step to obtain a construct with a tandem of 2 HS4 elements (Chung et al, 1997). This step was done both for the 5′ and 3′ of the cassette encompassing the CMV promoter, TTG Zeo and d2EGFP. The 2959 bp long chicken lysozyme MAR (Stief et al, 1989) was cloned 5′ and 3′ of the cassette encompassing the CMV promoter, TTG Zeo and d2EGFP. The 2614 bp long UCOE (Williams et al, 2005) was a NotI-KpnI fragment, excised from a human BAC clone (RP11-93D5), corresponding to nucleotide 29449 to 32063. This fragment was cloned 5′ of the CMV promoter. The STAR construct contained STAR7 and STAR67 5′ of the CMV promoter and STAR7 3′ of the cassette. These four constructs, as well as the control construct without flanking chromatin control DNA elements, were transfected to CHO-K1 cells. Selection was performed by 200 μg/ml Zeocin in the culture medium. Colonies were isolated, propagated and d2EGFP expression levels were measured. As shown in FIG. 37, constructs with all DNA elements resulted in the formation of d2EGFP expressing colonies. However, incorporation of 2×HS4 elements and the UCOE did not result in the formation of colonies that displayed higher d2EGFP expression levels, in comparison with the control colonies. In contrast, incorporation of the lysozyme MAR resulted in the formation of colonies that expressed d2EGFP significantly higher. The mean expression level induced by MAR containing constructs was four-fold higher than in the control colonies. Best results were obtained, however, by incorporating STAR 7 and 67 in the construct. An almost ten-fold increase in the mean d2EGFP expression level was observed, as compared to the control colonies. We conclude that other chromatin control DNA elements such as MARs can be used in the context of the selection system of the invention. However, the best results were obtained when STAR elements were used as chromatin control elements.

### Example 19

### Stringent Selection by Placing a Modified Zeocin Resistance Gene Behind an IRES Sequence

The previous examples (all from the incorporated '525 application) have shown a selection system where a sequence encoding a selectable marker protein is upstream of a sequence encoding a protein of interest in a multicistonic transcription unit, and wherein the translation initiation sequence of the selectable marker is non-optimal, and wherein further internal ATGs have been removed from the selectable marker coding sequence. This system results in a high stringency selection system. For instance the Zeo selection marker wherein the translation initiation codon is changed into TTG was shown to give very high selection stringency, and very high levels of expression of the protein of interest encoded downstream.

In another possible selection system the selection marker, e.g. Zeo, is placed downstream from an IRES sequence. This creates a multicistronic mRNA from which the Zeo gene product is translated by IRES-dependent initiation. In the usual d2EGFP-IRES-Zeo construct, the Zeo startcodon is the optimal ATG. It is therefore possible that changing the Zeo ATG startcodon into for instance TTG (referred to as IRES-TTG Zeo) may result in increased selection stringencies compared to the usual IRES-ATG Zeo.

Results

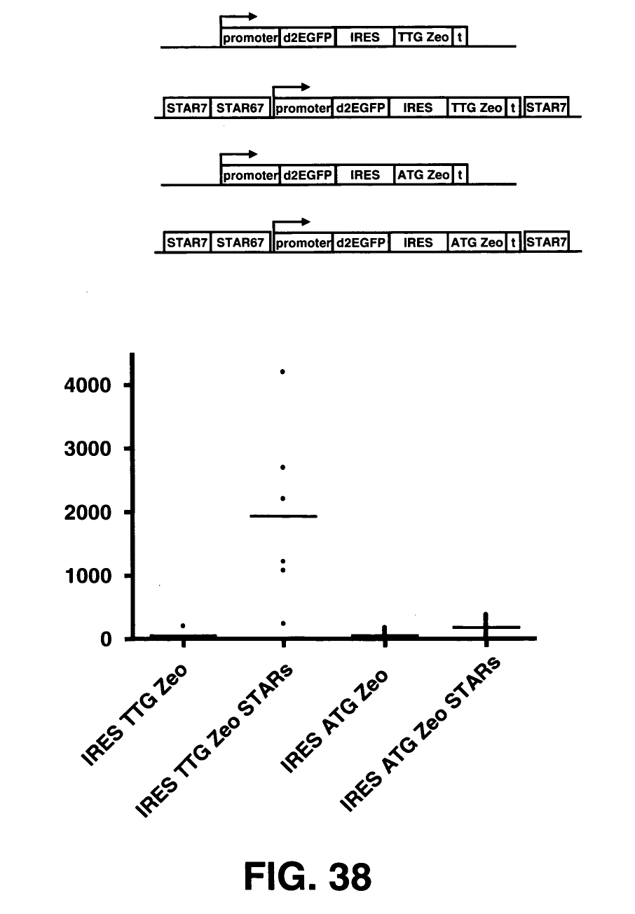

The used constructs are schematically shown in FIG. 38. The control construct consisted of a CMV promoter, the d2EGFP gene, an IRES sequence (the sequence of the used IRES (Rees et al, 1996) in this example was: GCCCCTCTCCCTCCCCCCCCCCTAACGTTACTGGCCGAAGCCGCTTGGAATAAGGCC GGTGTGCGTTTGTCTATATGTGATTTTCCACCATATTGCCGTCTTTTGGCAATGTGAG GGCCCGGAAACCTGGCCCTGTCTTCTTGACGAGCATTCCTAGGGGTCTTTCCCCTCTC GCCAAAGGAATGCAAGGTCTGTTGAATGTCGTGAAGGAAGCAGTTCCTCTGGAAGC TTCTTGAAGACAAACAACGTCTGTAGCGACCCTTTGCAGGCAGCGGAACCCCCCACC TGGCGACAGGTGCCTCTGCGGCCAAAAGCCACGTGTATAAGATACACCTGCAAAGG CGGCACAACCCCAGTGCCACGTTGTGAGTTGGATAGTTGTGGAAAGAGTCAAATGG CTCTCCTCAAGCGTATTCAACAAGGGGCTGAAGGATGCCCAGAAGGTACCCCATTGT ATGGGATCTGATCTGGGGCCTCGGTGCACATGCTTTACATGTGTTTAGTCGAGGTTA AAAAAACGTCTAGGCCCCCCGAACCACGGGGACGTGGTTTTCCTTTGAAAAACACG ATGATAAGCTTGCCACAACCCCGGGATA; SEQ. ID. NO. 127), and a TTG Zeo selection marker, i.e. the zeocin resistance gene with a TTG startcodon (‘d2EGFP-IRES-TTG Zeo’). The other construct was the same, but with a combination of STAR 7 and STAR 67 placed upstream of the expression cassette and STAR 7 downstream of the cassette (‘STAR7/67 d2EGFP-IRES-TTG Zeo STAR7’). Both constructs were transfected to CHO-K1 cells and selection was performed with 100 μg/ml Zeocin in the culture medium. Four colonies emerged after transfection with the control construct and six with the STAR containing construct. These independent colonies were isolated propagated before analysis of d2EGFP expression levels. As shown in FIG. 38, incorporation of STAR elements in the construct resulted in the formation of colonies with high d2EGFP expression levels. Of the control colonies without STAR elements (‘d2EGFP-IRES-TTG Zeo’) only one colony displayed some d2EGFP expression. The expression levels are also much higher than those obtained with other control constructs, containing the IRES with a normal Zeo with standard ATG startcodon, either with or without STAR elements (‘d2EGFP-IRES-ATG Zeo’ and ‘STAR 7/67 d2EGFP-IRES-ATG Zeo STAR7’; also in these ATG Zeo constructs there was an enhancing effect of the STAR elements, but these are modest as compared to the novel TTG Zeo variant).

These results show that placing a Zeo selection marker with a TTG startcodon downstream of an IRES sequence, in combination with STAR elements, operates well and establishes a stringent selection system.

From these data and the previous examples it will be clear that the marker can be varied along the same lines of the previous examples. For instance, instead of a TTG startcodon, a GTG startcodon can be used, and the marker can be changed from Zeo into a different marker, e.g. Neo, Blas, dhfr, puro, etc, all with either GTG or TTG as startcodon. The STAR elements can be varied by using different STAR sequences or different placement thereof, or by substituting them for other chromatin control elements, e.g. MAR sequences. This leads to improvements over the prior art selection systems having an IRES with a marker with a normal ATG startcodon.

As a non-limiting example, instead of the modified Zeo resistance gene (TTG Zeo) a modified Neomycin resistance gene is placed downstream of an IRES sequence. The modification consists of a replacement of the ATG translation initiation codon of the Neo coding sequence by a TTG translation initiation codon, creating TTG Neo. The CMV-d2EGF-RES-TTG Neo construct, either surrounded by STAR elements or not, is transfected to CHO-K1 cells. Colonies are picked, cells are propagated and d2EGFP values are measured. This (‘IRES-TTG Neo’) leads to improvement over the known selection system having Neo with an ATG startcodon downstream of an IRES (‘IRES-ATG Neo’). The improvement is especially apparent when the TTG Neo construct comprises STAR elements.

### REFERENCES

Boshart, M, Weber, F, Jahn, G, Dorsch-Hasler, K, Fleckenstein, B, and Schaffner, W. (1985) A very strong enhancer is located upstream of an immediate early gene of human cytomegalovirus Cell 41, 521-530.
Chung J H, Whiteley M, and Felsenfeld G. (1993) A 5′ element of the chicken beta-globin domain serves as an insulator in human erythroid cells and protects against position effect in Drosophila. Cell 74: 505-514.
Chung J H, Bell A C, Felsenfeld G. (1997). Characterization of the chicken beta-globin insulator. Proc Natl Acad Sci USA 94: 575-580.
Das, G C, Niyogi, S K, and Salzman, N P. (1985) SV40 promoters and their regulation Prog Nucleic Acid Res Mol Biol 32, 217-236.
Dumas, P, Bergdoll, M., Cagnon, C and Masson J M. 1994. Crystal structure and site-directed mutagenesis of a bleomycin resistance protein and their significance for drug sequestering. EMBO J 13, 2483-2492.
Gill D R, Smyth S E, Goddard C A, Pringle I A, Higgins C F, Colledge W H, and Hyde S C. (2001) Increased persistence of lung gene expression using plasmids containing the ubiquitin C or elongation factor 1α promoter. Gene Therapy 8: 1539-1546.
Gossen M, and Bujard H. (1992) Tight control of gene expression in mammalian cells by tetracycline-responsive promoters. Proc Natl Acad Sci USA 89: 5547-5551.
Graham F O, Smiley J, Russell W and Nairn R. (1977). Characteristics of a human cell line transformed by DNA from human adenovirus type 5. J. Gen. Virol. 36, 59-72.
Huls G A, Heijnen I A F M, Cuomo M E, Koningsberger J C, Wiegman L, Boel E, van der Vuurst-de Vries A-R, Loyson S A J, Helfrich W, van Berge Henegouwen G P, van Meijer M, de Kruif J, Logtenberg T. (1999). A recombinant, fully human monoclonal antibody with antitumor activity constructed from phage-displayed antibody fragments. Nat Biotechnol. 17, 276-281.
Jones D, Kroos N, Anema R, Van Montfort B, Vooys A, Van Der Kraats S, Van Der Helm E, Smits S, Schouten J, Brouwer K, Lagerwerf F, Van Berkel P, Opstelten D-J, Logtenberg T, Bout A (2003) High-level expression of recombinant IgG in the human cell line PER.C6. Biotechnol. Prog. 19: 163-168.
Kaufman, R J. (2000) Overview of vector design for mammalian gene expression Mol Biotechnol 16, 151-160.
Kaufman, R J, and Sharp, P A. (1982) Construction of a modular dihydrofolate reductase cDNA gene: analysis of signals utilized for efficient expression Mol Cell Biol 2, 1304-1319.
Kellum R, and Schedl P. (1991) A position-effect assay for boundaries of higher order chromosomal domains. Cell 64: 941-950.
Kim S J, Kim Ns, Ryu C J, Hong H J, Lee G M. 1998. Characterization of chimeric antibody producing CHO cells in the course of dihydrofolate reductase-mediated gene amplification and their stability in the absence of selective pressure. Biotechnol Bioeng 58: 73-84.
Kozak M. (1986) Point mutations define a sequence flanking the AUG initiator codon that modulates translation by eukaryotic ribosomes. Cell 44: 283-292.
Kozak M. (1987) An analysis of 5′-noncoding sequences from 699 vertebrate messenger RNAs. Nucleic Acids Res. 15: 8125-8148.
Kozak M. (1989) Context effects and inefficient initiation at non-AUG codons in eucaryotic cell-free translation systems. Mol Cell Biol. 9: 5073-5080.
Kozak M. (1990) Downstream secondary structure facilitates recognition of initiator codons by eukaryotic ribosomes. Proc Natl Acad Sci USA 87:8301-8305.
Kozak M. (1997) Recognition of AUG and alternative initiator codons is augmented by G in position +4 but is not generally affected by the nucleotides in positions +5 and +6. EMBO J. 16: 2482-2492.
Kozak M. (2002) Pushing the limits of the scanning mechanism for initiation of translation. Gene 299: 1-34.
Kwaks T H, Barnett P, Hemrika W, Siersma T, Sewalt R G, Satijn D P, Brons J F, van Blokland R, Kwakman P, Kruckeberg A L, Kelder A, Otte A P. (2003) Identification of anti-repressor elements that confer high and stable protein production in mammalian cells. Nat Biotechnol 21, 553-558. Erratum in: Nat Biotechnol 21, 822 (2003).
Lopez de Quinto, S, and Martinez-Salas, E. (1998) Parameters influencing translational efficiency in aphthovirus IRES-based bicistronic expression vectors Gene 217, 51-6.
Phi-Van L, Von Kreis J P, Ostertag W, and Strätling W H. (1990) The chicken lysozyme 5′ matrix attachment region increases transcription from a heterologous promoter in heterologous cells and dampens position effects on the expression of transfected genes. Mol. Cell. Biol. 10: 2302-2307.
Martinez-Salas, E. (1999) Internal ribosome entry site biology and its use in expression vectors Curr Opin Biotechnol 10, 458-64.
McBurney, M W, Mai, T, Yang, X, and Jardine, K. (2002) Evidence for repeat-induced gene silencing in cultured Mammalian cells: inactivation of tandem repeats of transfected genes Exp Cell Res 274, 1-8.
Mizuguchi, H, Xu, Z, Ishii-Watabe, A, Uchida, E, and Hayakawa, T. (2000) IRES-dependent second gene expression is significantly lower than cap-dependent first gene expression in a bicistronic vector Mol Ther 1, 376-82.
Rees, S, Coote, J, Stables, J, Goodson, S, Harris, S, and Lee, M G. (1996) Bicistronic vector for the creation of stable mammalian cell lines that predisposes all antibiotic-resistant cells to express recombinant protein Biotechniques 20, 102-104, 106, 108-110.
Schorpp, M, Jager, R, Schellander, K, Schenkel, J, Wagner, E F, Weiher, H, and Angel, P. (1996) The human ubiquitin C promoter directs high ubiquitous expression of transgenes in mice Nucleic Acids Res 24, 1787-8.
Stief A, Winter D M, Stratling W H, Sippel A E (1989) A nuclear DNA attachment element mediates elevated and position-independent gene activity. Nature 341: 343-345.
Van der Vlag, J, den Blaauwen, J L, Sewalt, R G, van Driel, R, and Otte, A P. (2000) Transcriptional repression mediated by polycomb group proteins and other chromatin-associated repressors is selectively blocked by insulators J Biol Chem 275, 697-704.
Venkatesan, A, and Dasgupta, A. (2001) Novel fluorescence-based screen to identify small synthetic internal ribosome entry site elements Mol Cell Biol 21, 2826-37.
West A G, Gaszner M, Felsenfeld G (2002) Insulators: many functions, many mechanisms. Genes Dev. 16: 271-288.
Whitelaw, E, Sutherland, H, Kearns, M, Morgan, H, Weaving, L, and Garrick, D. (2001) Epigenetic effects on transgene expression Methods Mol Biol 158, 351-68.
Williams S, Mustoe T, Mulcahy T, Griffiths M, Simpson D, Antoniou M, Ivine A, Mountain A, Crombie R (2005) CpG-island fragments from the HNRPA2B1/CBX3 genomic locus reduce silencing and enhance transgene expression from the hCMV promoter/enhancer in mammalian cells. BMC Biotechnol. 5:17.

1.-60. (canceled)

61. A deoxyribonucleic acid (DNA) molecule comprising: a multicistronic transcription unit comprising at least one coding sequence coding for both
i) a polypeptide of interest, and
ii) a selectable marker polypeptide functional in a eukaryotic host cell,
wherein the polypeptide of interest has a translation initiation sequence separate from that of the selectable marker polypeptide,
wherein the at least one coding sequence for the polypeptide of interest is upstream from the at least one coding sequence for the selectable marker polypeptide in said multicistronic transcription unit,
wherein an internal ribosome entry site (IRES) is present downstream from the at least one coding sequence for the polypeptide of interest and upstream from the at least one coding sequence for the selectable marker polypeptide, and
wherein the coding sequence coding for the selectable marker polypeptide comprises a translation start sequence selected from the group consisting of:
a) a GTG start codon;
b) a TTG start codon;
c) a CTG start codon;
d) a ATT start codon; and
e) a ACG start codon.

62. The DNA molecule of claim 61, wherein the translation start sequence for the selectable marker polypeptide comprises a GTG start codon.

63. The DNA molecule of claim 61, wherein the translation start sequence for the selectable marker polypeptide comprises a TTG start codon.

64. The DNA molecule of claim 61, wherein the selectable marker polypeptide provides resistance against lethal or growth-inhibitory effects of a selection agent.

65. The DNA molecule of claim 64, wherein said selection agent is selected from the group consisting of zeocin, puromycin, blasticidin, hygromycin, neomycin, methotrexate, methionine sulphoximine and kanamycin.

66. The DNA molecule of claim 64, wherein the selection agent is zeocin.

67. The DNA molecule of claim 61, wherein the selectable marker polypeptide further comprises a mutation that reduces the activity of the selectable marker polypeptide compared to its wild-type counterpart.

68. The DNA molecule of claim 61, wherein the coding sequence of the polypeptide of interest comprises an optimal translation start sequence comprising the sequence (A/G)CCATGG, wherein the start codon is underlined.

69. An expression cassette comprising the DNA molecule of claim 61, said expression cassette comprising a promoter upstream of said multicistronic expression unit and a transcription termination sequence downstream of the multicistronic expression unit, wherein said expression cassette is functional in a eukaryotic host cell for initiating transcription of the multicistronic expression unit.

70. The expression cassette of claim 69, further comprising at least one chromatin control element selected from the group consisting of a matrix or scaffold attachment region (MAR/SAR), an insulator sequence, an universal chromatin opening element (UCOE), and an anti-repressor (STAR) sequence.

71. The expression cassette of claim 70, wherein said at least one chromatin control element is an anti-repressor sequence selected from the group consisting of:
a) any one of SEQ. ID. NO. 1 through SEQ. ID. NO. 66 and
b) the complement of a).

72. The expression cassette of claim 70, wherein said expression cassette comprises SEQ. ID. NO. 66 positioned upstream of the promoter that drives transcription of the multicistronic expression unit.

73. The expression cassette of claim 70, wherein said multicistronic expression unit is flanked on both sides by at least one anti-repressor sequence chosen from the group consisting of:
a) any one of SEQ. ID. NO. 1 through SEQ. ID. NO. 65 and
b) the complement of a).

74. The expression cassette of claim 70, comprising: 5′—anti-repressor sequence A—anti-repressor sequence B—promoter—multicistronic gene encoding the polypeptide of interest and downstream thereof the functional selectable marker protein—transcription termination sequence—anti-repressor sequence C—3′,
wherein anti-repressor sequences A and C may be the same or different and are any one of SEQ. ID. NO. 1 through SEQ. ID. NO. 65, wherein anti-repressor sequence B is SEQ. ID. NO. 66.

75. The expression cassette of claim 74, wherein anti-repressor sequences A and C are SEQ. ID. NO. 7.

76. The expression cassette of claim 69, wherein the polypeptide of interest is a portion of a multimeric protein.

77. The expression cassette of claim 76, wherein the polypeptide of interest is selected from the group consisting of an immunoglobulin light chain and an immunoglobulin heavy chain.

78. A host cell comprising the DNA molecule of claim 61.

79. A host cell comprising the expression cassette of claim 69.

80. A host cell comprising the expression cassette of claim 71.

81. The host cell of claim 78, wherein the host cell is a mammalian cell.

82. The host cell of claim 79, wherein the host cell is a mammalian cell.

83. The host cell of claim 81, wherein the mammalian cell is a Chinese hamster ovary (CHO) cell.

84. The host cell of claim 82, wherein the mammalian cell is a Chinese hamster ovary (CHO) cell.

85. A method of generating a host cell able to express a polypeptide of interest, said method comprising the steps of:
a) introducing into a plurality of precursor cells the DNA molecule of claim 61, and
b) culturing the plurality of precursor cells under conditions suitable for expression of the selectable marker polypeptide, and
c) selecting at least one host cell expressing the polypeptide of interest.

86. A method of generating a host cell able to express a polypeptide of interest, said method comprising the steps of:
a) introducing into a plurality of precursor cells the expression cassette of claim 69, and
b) culturing the plurality of precursor cells under suitable conditions for expression of the selectable marker polypeptide, and
c) selecting at least one host cell expressing the polypeptide of interest.

87. A method of expressing a polypeptide of interest, comprising culturing a host cell comprising the expression cassette of claim 69, and expressing the polypeptide of interest from the expression cassette.

88. The method according to claim 87, further comprising harvesting the polypeptide of interest.

89. A method of expressing a polypeptide of interest, comprising culturing a host cell comprising the expression cassette of claim 70 and expressing the polypeptide of interest from the expression cassette.

90. The method according to claim 89, further comprising harvesting the polypeptide of interest.

91. A method of expressing a polypeptide of interest, comprising culturing a host cell comprising the expression cassette of claim 71 and expressing the polypeptide of interest from the expression cassette.

92. The method according to claim 91, further comprising harvesting the polypeptide of interest.
